(19) United States
(12) Patent Application Publication
(10) Pub. No.: US 2025/0009901 A1
(43) Pub. Date: Jan. 9, 2025

(54) COMPOSITION COMPRISING A COMBINATION OF IMMUNE CHECKPOINT INHIBITOR AND ANTIBODY-AMATOXIN CONJUGATE FOR USE IN CANCER THERAPY

(71) Applicant: Heidelberg Pharma Research GmbH, Ladenburg (DE)

(72) Inventors: Michael Kulke, Ladenburg (DE); Torsten Hechler, Ladenburg (DE); Andreas Pahl, Ladenburg (DE); Christoph Müller, Ladenburg (DE); Susanne Werner-Simon, Ladenburg (DE)

(73) Assignee: Heidelberg Pharma Research GmbH, Ladenburg (DE)

(21) Appl. No.: 18/825,191

(22) Filed: Sep. 5, 2024

Related U.S. Application Data
(63) Continuation of application No. 17/518,911, filed on Nov. 4, 2021.

(30) Foreign Application Priority Data
Nov. 4, 2020 (EP) 20205794.9
Apr. 13, 2021 (EP) 21168014.5

(51) Int. Cl. A61K 47/68

(52) U.S. Cl. CPC A61K 47/6851; A61K 47/6811; A61K 47/6849

(57) ABSTRACT

The present application relates to a composition comprising (a) at least one immune checkpoint inhibitor and (b) at least one conjugate, wherein said conjugate is comprising (i) a target binding moiety, (ii) at least one amatoxin, and (iii) optionally at least one linker connecting said target binding moiety with said at least one amatoxin. The present application further relates to said composition for use in treating a patient having a cancer, and to a pharmaceutical formulation comprising said composition and additional excipients, as well as to methods of producing and using said composition (FIG. 1).

### CROSS-REFERENCE TO RELATED APPLICATIONS

This application is a continuation of U.S. application Ser. No. 17/518,911, filed on Nov. 4, 2021, and which claims the benefit of priority to European Patent Application No. 21168014, filed on Apr. 13, 2021, and to European Patent Application No. 20205794, filed on Nov. 4, 2020. The content of each of these applications is hereby incorporated by reference in their entirety.

### FIELD OF THE INVENTION

The present application relates to the field of cancer immunotherapy. In particular, the present application relates to a composition comprising (a) at least one immune checkpoint inhibitor and (b) at least one conjugate, wherein said conjugate is comprising (i) a target binding moiety, (ii) at least one amatoxin, and (iii) optionally at least one linker connecting said target binding moiety with said at least one amatoxin. The present application further relates to said composition for use in treating a patient having a cancer, and to a pharmaceutical formulation comprising said composition and additional excipients, as well as to methods of producing and using said composition.

### BACKGROUND

Regulation of the immune system is mediated by a variety of mechanisms, including regulatory cells of the innate and adaptive immune system, like regulatory T cells (Treg), myeloid-derived suppressor cells (MDSCs) and M2-type macrophages; regulatory cytokines, such as IL-10 and TGF-β; and immune checkpoints that control T cell activation. Such cells and molecules can be exploited as a mechanism of immune subversion during development of cancer and chronic infectious diseases. However, on the other hand, one of the most successful cancer treatment strategies developed to date has been based on immunotherapy. This treatment approach aims to enhance the immune response of the host or patient to the different progression stages of tumors, with fewer off-target outcomes compared with chemotherapy drugs or other treatment modalities that destroy cancer cells directly (Singh et al, 2020).

Immune checkpoints are receptors on the cell membrane of T lymphocytes which modulate the immune reactivity of said cells. There are anti-inflammatory (suppressing) and pro-inflammatory (activating) immune checkpoints which are expressed on the cell membrane of T cells, and which are interacting with respective ligands, either soluble or cell-bound ligands. Tumor cells often activate anti-inflammatory immune checkpoint pathways via respective ligands that suppress anti-tumor immune responses, thus evading immuno-surveillance and progressing tumor growth. Immune checkpoint inhibitors (ICIs) are agents, in particular monoclonal antibodies, which bind to anti-inflammatory immune checkpoints or their ligands and can interrupt this tumor suppression strategy by reactivating the immune system and, hence, reestablishing its capacity to combat tumors. ICIs have been shown to be clinically effective in a variety of tumor types (Dyck and Mills, 2017; Darwin et al, 2018).

Two of the most widely studied and used ICI targets for drug development have been the PD-1/PD-L1-PD-L2 and the CTLA4/CD80-CD86 receptor-ligand signaling pathways. Both pathways result in inhibition of T-cell activation, proliferation, and survival. Whereas the CTLA-4 receptor is predominantly expressed in T cells, PD-1 is expressed in activated T cells, B cells, and certain myeloid cells. Moreover, whereas CTLA-4 works in the priming phase of T-cell activation and limits early T-cell activation, PD-1 works later, during the effector phase, mostly in peripheral tissues where T cells encounter PD-1 ligands (Dyck and Mills, 2017).

Two signals are required for T-cell activation: recognition of peptide antigen presented by the major histocompatibility complex (MHC) as first signal, and costimulation through CD28 following binding to CD80 or CD86 expressed by antigen-presenting cells (APCs) as second signal (FIG. 2). CTLA-4, one of the first inhibitory receptors identified as playing a role in suppression of T-cell responses, is structurally similar to CD28 and binds to CD80 and CD86 at a higher affinity than CD28. It has been suggested that CTLA-4 expression interferes with T-cell activation by reducing the CD28-mediated costimulatory (second) signal, leading to T cell anergy; anergic T cells have limited effector function (Dyck and Mills, 2017). CTLA-4 expression and function is intrinsically linked with T-cell activation; CTLA-4 is immediately upregulated following T cell receptor (TCR) engagement (signal 1). Blocking CTLA-4 in vivo has been shown to inhibit CTLA-4 binding to CD80/86 and to promote anti-tumoral immunity by inhibiting Treg cells and enhancing effector T-cell function (Wei et al, 2018).

The Programmed Cell Death Protein 1 (PD-1) molecule consists of an intracellular domain which has potential phosphorylation sites within an immune tyrosine-based inhibitory motif (ITIM) and an immune receptor inhibitory tyrosine-based switch motif (ITSM), a hydrophobic transmembrane region, and an extracellular IgV domain (Li et al, 2016). An activated switch motif (ITSM) is required for the inhibitory effect of PD-1 on active T cells; PD-1 ligand binding leads to recruitment of the tyrosine phosphatase SHP-2 to the ITSM motif and thus interferes with TCR downstream signalling. In addition, PD-1 ligand binding leads to interference with signalling molecules such as PIP-3 kinase and Ras which are important for T-cell proliferation, cytokine secretion and metabolism, and furthermore induces metabolic alterations in T effector cells and promotes induction of Treg cells. PD-1 ligand binding can also lead to T-cell exhaustion.

PD-1 has been detected on T cells, Tregs, exhausted T cells, B cells, activated monocytes, dendritic cells (DCs), natural killer (NK) cells, natural killer T (NKT) cells, epithelial cells, and tumor cells (Li et al, 2016). PD-1 expression on T cells is induced by antigen stimulation. PD-1 mainly exerts its inhibitory effect on T cells in the periphery. Two ligands of PD-1 have been identified, PD-L1 (CD274) and PD-L2 (CD273). In cancer, tumor cells and myeloid cells are thought to be the main cell types mediating T-cell suppression through PD-1 binding (Li et al, 2016; Dyck and Mills, 2017).

In addition to CTLA-4 and PD-1, recent studies have identified further immune checkpoint targets like lymphocyte activation gene-3 (LAG-3), T cell immunoglobulin and mucindomain containing-3 (TIM-3), T cell immunoglobulin and ITIM domain (TIGIT), V-domain Ig suppressor of T cell activation (VISTA), CD96, and BTLA (CD272) (Qin et al, 2019).

LAG-3 is usually expressed on activated CD4-positive and CD8-positive T cells, Tregs, a subpopulation of natural killer (NK) cells, B cells, and plasmacytoid dendritic cells (pDCs). Studies have indicated that LAG-3 signaling plays a negative regulatory role in T helper 1 (Th1) cell activation, proliferation and cytokine secretion. During tumorigenesis and cancer progression, tumor cells exploit this pathway to escape from immune surveillance. MHC-II, galectin-3, LSECtin, a-synuclein, and fibrinogen-like protein 1 (FGL1) have been described to interact with LAG-3.

TIM-3, also called hepatitis A virus cellular receptor 2 (HAVCR2) belongs to the Ig super-family with an N-terminal Ig variable region (IgV)-like domain, a membrane-proximal mucin-like domain containing sites for O-linked glycosylation (glycosylated mucin domain), a single transmembrane region and a C-terminal cytoplasmic tail. Expression of TIM-3 is not limited to T cells, it is known to be expressed on different types of immune cells, including B cells, Tregs, NK cells, DCs, monocytes, and macrophages. Four distinct ligands have been reported to bind to the IgV domain of TIM-3, including galectin-9, high-mobility group protein BI (HMGB1), carcinoembryonic antigen cell adhesion molecule 1 (Ceacam-1), and phosphatidyl serine (PtdSer), wherein galectin-9 and HMGBlare soluble ligands, while Ceacam-1 and PtdSer belong to surface ligands. The engagement of TIM-3 with galectin-9 has been described to trigger intracellular calcium flux in Th1 cells, inducing cell death; galectin-9 also induced apoptosis of TIM-3- and CD8-positive T cells in colon cancer. The interaction between HMGB1 and TIM-3 mainly has an impact on innate immune response.

The TIGIT protein includes an extracellular IgV region, a transmembrane domain, and a cytoplasmic tail that harbors an ITIM and an immunoglobulin tail tyrosine (ITT)-like phosphorylation motif. The expression of TIGIT has been demonstrated to be tightly restricted to lymphocytes, mainly on T cell subsets (including Tregs and memory T cells) and NK cells. TIGIT binds two ligands, namely CD155 (PVR or Necl-5) and CD112 (nectin-2, also known as PRR2 or PVRL2) with different affinity. TIGIT exerts its immunosuppressive effects by competing for ligands with other counterparts like CD266 (DNAM-1). CD226 delivers a positive co-stimulatory signal, while TIGIT delivers inhibitory signals into the T cell (Qin et al, 2019).

Almost 4,000 active immuno-oncology-related therapeutic agents are currently in the global drug development pipeline, and the worldwide number of clinical trials exploring PD-1/PD-L1 blockade alone has already reached 2,250. Since approval of ipilimumab (anti-CTLA-4) in 2011 in US for the treatment of metastatic melanoma, the number of indications for which ICIs have been approved has increased to well over 20 (Taams and de Gruijl, 2020). The US Food and Drug Administration (FDA) has so far approved two classes of ICIs that show promising therapeutic outcomes: CTLA-4 and PD-1 (CD279) and its ligands, PD-L1 (CD274, B7-H1) and PD-L2 (CD273, B7-DC), with others currently in clinical trials (Singh et al, 2020).

Therapeutic monoclonal antibodies which have been approved by regulatory authorities as immune checkpoint inhibitors are including Ipilimumab directed against CTLA-4; Nivolumab and Pembrolizumab directed against PD-1; and Atezolizumab, Avelumab, Durvalumab and Cemiplimab directed against PD-L1 (Singh et al, 2020).

The tumor types for which immune checkpoint blockade therapies have been approved by regulatory authorities are including melanoma, squamous and non-squamous non-small cell lung cancer, metastatic small cell lung cancer, renal cell carcinoma, Hodgkin lymphoma, urothelial carcinoma, head and neck squamous cell carcinoma, Merkel cell carcinoma, hepatocellular carcinoma, gastric and gastroesophageal carcinoma, metastatic colorectal cancer, primary mediastinal B cell lymphoma, recurrent or metastatic cervical cancer, and metastatic cutaneous squamous cell carcinoma (Wei et al, 2018; Singh et al, 2020).

In addition to monotherapies with CTLA-4- or PD-1-blocking antibodies, combination therapies comprising. e.g., Ipilimumab and Nivolumab have been used. Furthermore, ICIs have been employed in combination with cancer vaccines typically consisting of a source of cancer antigens and adjuvants that activate innate immune cells like dendritic cells; cancer vaccines aim to generate tumor-specific T cells that kill tumor cells via secretion of IFN-γ or lytic granules. ICIs have also been used in combination with radiotherapy and in combination with histone deacetylase inhibitors, which can induce direct tumor cytotoxicity and improve tumor immunogenicity in some cancer types.

ICIs have been regarded as one of the most important developments in cancer therapy over the past decade. However, in particular in monotherapeutic approaches with individual ICIs, a significant part of patients did not respond (Dyck and Mills, 2017). Furthermore, the emergence of a new spectrum of immune-related adverse events (irAEs) associated with this approach has also been noted (Martins et al, 2019), suggesting a need for high cancer cell specificity and selectivity of this class of therapeutic agents.

One of the major aspects for clinical success of anti-cancer agents has turned out to be their ability to induce immunogenic cell death (ICD), i.e., a cell death modality that does stimulate an immune response against dead-cell antigens, in particular deriving from cancer cells, as compared to non-immunogenic cancer cell death, for example by apoptosis. ICD involves changes in the composition of the cell surface as well as the release of soluble mediators; these signals operate on a series of receptors expressed by dendritic cells, thus stimulating their presentation of tumor antigens to T cells (Kroemer et al, 2013). This cell death modality originally has been observed and studied in the context of anti-cancer chemotherapy.

ICD has been found to be characterized by a combination of alterations in the composition of the plasma membrane of dying cells and in the composition of the microenvironment (FIG. 3). These alterations result from premortem stress and subsequent cellular disintegration. ICD is obligatorily preceeded by two types of stress, which are endoplasmic reticulum (ER) stress and autophagy as an adaptive response to stress. As a result of ER stress due to, e.g., chemotherapy, calreticulin (CRT), whose largest fraction is normally secluded in the ER lumen, heat shock proteins and other ER proteins are exposed at the outer surface of the plasma membrane. As a result of and depending from autophagy, adenosine triphosphate (ATP) is secreted from the dying cell. As a result of cell death-associated cellular disintegration, the non-histone chromatin protein high-mobility group box 1 (HMGB1) is released into the microenvironment.

The exposure of CRT and other ER proteins at the cell surface, the secretion of ATP, and the release of HMGB1 are hallmarks of ICD as opposed to non-immunogenic cell death. CRT, ATP and HMGB1 interact with CD91 (low density lipoprotein receptor-related protein 1, LRP1), P2RX7 (purinergic receptor) and TLR4 (Toll-like receptor 4) receptors, respectively, which are expressed by dendritic cells and promote engulfment of dying cells, production of cytokines like IL-1β, and presentation of tumor antigens. The patient's dying cancer cells, hence, operate as a vaccine that stimulates a tumor-specific immune response, characterized by DC recruitment, increased number and activity of T lymphocytes as well as increased ratio of cytotoxic CD8-positive T lymphocytes (CTLs) over regulatory T cells (Tregs) within the tumor, which in turn can control residual cancer cells (Kroemer et al, 2013).

Distinct chemotherapeutic agents are not equivalent in their capacity to induce ICD. In a study testing 24 distinct cytotoxic chemotherapeutics on cancer cells in vivo, it was observed that only four of them (three anthracyclins and oxaliplatin) did elicit protective anti-cancer immune responses, although all agents induced apopotosis (Obeid et al, 2007).

Therefore, there is a need for developing therapeutic agents or compositions combining the immune checkpoint inhibitory effect of ICIs with a high cancer cell specificity and selectivity as well as a high capacity for inducing ICD.

Amatoxins are cyclic peptides composed of 8 amino acids that are found in Amanita phalloides mushrooms (see FIG. 1). Amatoxins specifically inhibit the DNA-dependent RNA polymerase II of mammalian cells, and thereby also the transcription and protein biosynthesis of the affected cells. Inhibition of transcription in a cell causes stop of growth and proliferation. Though not covalently bound, the complex between amanitin and RNA-polymerase II is very tight (KD=3 nM). Dissociation of amanitin from the enzyme is a very slow process, thus making recovery of an affected cell unlikely. When the inhibition of transcription lasts sufficiently long, the cell will undergo programmed cell death (apoptosis).

Antibody drug conjugates (ADCs) comprising amatoxins (antibody-targeted amatoxin conjugates) and tumor antigen-specific antibodies, antibody fragments or derivatives or antibody-like proteins, have been described (WO2010/115629A2, WO2016/142049A1, WO2017/149077A1).

As shown in the present application, the inventors unexpectedly found that amatoxin-based ADCs comprising tumor antigen-specific antibody as target binding moiety induces immuno-genic cell death. Surprisingly, the inventors have further observed a synergistic effect of an amatoxin-based ADCs and an immune checkpoint inhibitor with regard to their tumor-cell killing activities in vivo. These results were unexpected, as neither amatoxins alone nor amatoxin-based ADCs have been shown to have such beneficial effects before.

### SUMMARY OF THE INVENTION

In view of the prior art, it was hence one object of the present invention to provide a pharmaceutical composition comprising
(a) at least one immune checkpoint inhibitor and
(b) at least one conjugate, wherein said conjugate is comprising
   (i) a target binding moiety,
   (ii) at least one amatoxin, and
   (iii) optionally at least one linker connecting said target binding moiety with said at least one amatoxin.

It was one further object of the present invention to provide a composition for use in the treatment of cancer or chronic infectious disease, said composition comprising
(a) at least one immune checkpoint inhibitor and
(b) at least one conjugate, wherein said conjugate is comprising
   (i) a target binding moiety,
   (ii) at least one amatoxin, and
   (iii) optionally at least one linker connecting said target binding moiety with said at least one amatoxin.

It was one further object of the present invention to provide a pharmaceutical formulation comprising said composition (for use), and further comprising one or more pharmaceutically acceptable buffers, surfactants, diluents, carriers, excipients, fillers, binders, lubricants, glidants, disintegrants, adsorbents, and/or preservatives.

These and further objects are met with methods and means according to the independent claims of the present invention. The dependent claims are related to specific embodiments.

The invention and general advantages of its features will be discussed in detail below.

### DESCRIPTION OF THE FIGURES

FIG. 1. Structural formulae of various amatoxins. The numbers in bold type (1 to 8) designate the standard numbering of the eight amino acids forming the amatoxin. The standard designations of the atoms in amino acids 1, 3, and 4 are also shown (Greek letters α to γ, Greek letters α to δ, and numbers from 1′ to 7′, respectively).

FIG. 2. Principal concept of immunogenic cell death. As a result of premortem endoplasmic reticulum stress and autophagy, cancer cells responding to ICD inducers expose CRT on the outer leaflet of their plasma membrane at a preapoptotic stage, and secrete ATP during apoptosis. In addition, cells undergoing ICD release the nuclear protein HMGB1 as their membranes become permeabilized during secondary necrosis. CRT, ATP, and HMGB1 bind to the receptors CD91, P2RX7, and TLR4, respectively. This facilitates the recruitment of DCs into the tumor bed (stimulated by ATP), the engulfment of tumor antigens by DCs (stimulated by CRT), and optimal antigen presentation to T cells (stimulated by HMGB1). Altogether, these processes result in a potent IL-1β- and IL-17-dependent, IFN-γ-mediated immune response involving both γδT cells and CTLs. ATP, adenosine triphosphate; CRT, calreticulin; CTL, cytotoxic CD8+ T lymphocyte; DC, dendritic cell; HMGB1, high-mobility group box 1; IFN, interferon; IL, interleukin; TLR, Toll-like receptor. (From Kroemer et al. 2013).

FIG. 3. Principal concept of immune checkpoint inhibition. MHC presentation of peptide antigen to the T cell receptor provides a first signal for T cell activation (1). Through interaction between CD80 on the antigen-presenting cell and CD28 on the T cell, the T cell receives a second, costimulatory activation signal (2). CTLA-4 competes with CD28 for binding to CD80, and delivers an inhibitory signal to the T cell (3). PD-1 receptor on the T cell binds to PD-L1, delivering an inhibitory signal to the T cell (4). Tumor cells employ the use of these mechanisms in order to prevent T cells from clearing malignant cells. By using inhibitors (CTLA-4, PD-1, or PD-L1 inhibitors) that prevent this interaction from occurring, T cells remain active after identifying tumor cells and can clear them from the host. CD, cluster of differentiation; CTLA-4, cytotoxic T lymphocyte associated antigen-4; MHC, major histocompatibility complex; PD-1, programmed cell death protein-1; PD-L1, PD ligand-1; TCR, T-cell receptor. (Fig. from Sambi et al. 2019).

FIG. 4. Immunogenic cell death induced by antibody-targeted amatoxin conjugates (ATACs). The Her2-positive cell line BT474 and the CD79b-positive cell line BJAB, respectively, were exposed to different compounds. Immunogenic cell death (ICD) markers calreticulin (CRT), adenosin tri-phosphate (ATP), and high-mobility group box 1 protein (HMGB1) were determined. Amanitin-conjugated ADCs induce the secretion of ICD markers in a target-dependent manner. (A, B, C) Her2-positive cell line BT474, and (D, E, F) CD79b-positive cell line BJAB were exposed to no compound (first bar), 100 nM Maytansine (second bar), 100 nM amanitin (third bar), 50 nM Anti-Her2-amanitin conjugate (fourth bar) and 50 nM Anti-CD79b-amanitin conjugate (fifth bar), respectively; and (A), (D) CRT-exposing (positive) cells; (B), (E) ATP secretion; and (C), (F) HMGB1 release were assessed.

FIG. 5. Synergistic action of Avelumab and Anti-CD19-Amatoxin conjugate in the presence of peripheral blood mononuclear cells (PBMCs). Results of anti-tumoral studies using anti-PD-L1 antibody Avelumab (20 mg/kg, i.v., days 0, 3, 6, 8, 10, 13), or anti-CD19-amatoxin conjugate (ATAC, 0.1 and 0.3 mg/kg, respectively; single dose i.v. day 0), or a combination of Avelumab (20 mg/kg) and ATAC (0.1 and 0.3 mg/kg, respectively), in vivo in a Raji xenograft mice model system. Tumor volume was assessed at various time points after tumor cell inoculation.

FIG. 6. Synergistic action of Avelumab and Anti-CD19-Amatoxin conjugate in the absence or presence of peripheral blood mononuclear cells (PBMCs). Results of anti-tumoral studies using anti-PD-L1 antibody Avelumab (20 mg/kg, i.v., days 0, 3, 6, 8, 10, 13), or anti-CD19-amatoxin conjugate (ATAC, 0.3 mg/kg; single dose i.v. day 0), or a combination of Avelumab (20 mg/kg) and ATAC (0.3 mg/kg), in the absence of PBMCs (groups 01-04) and in the presence of PBMCs (groups 05-08), respectively, in vivo in a Raji xenograft mice model system. Tumor volume was assessed at various time points after tumor cell inoculation.

FIG. 7. Overview of cytotoxic potency of anti-HER2 ATACs in different cell lines. Anti-HER2-LALA-D265C antibodies conjugated to linker-comprising amatoxins XIXa, XVIIIa, XIIIa, XIIa, XXIIa, XXIa (the corresponding conjugated amatoxins coupled to the respective anti-HER2-LALA-D265C antibody are designated XIXb, XVIIIb, XIIIb, XIIb, XXIIb, XXIb wherein the anti-HER2 antibody corresponds to the antibody in said formulae.).

FIG. 8. Efficacy of anti-Her2 ATACs in a s.c. JIMT-1 Xenograft model. For the Jimt-1 xenograft model female NMRI nude mice were inoculated with 5×10⁶ Jimt-1 breast cancer cells (Mol Cancer Ther. 2004 December; 3 (12): 1585-92) per mouse subcutaneously in the right flank. At a mean tumor vol. of ˜120 mm³, animals were allocated to respective experimental groups on day 0. On the same day the animals received a single intravenous dose of amanitin based anti-Her2 antibody drug conjugates (ADCs) as indicated. Tumor volume and body weight were determined twice per week.

FIG. 9. ATAC treatment in vivo leads to immunity towards tumor re-challenge. NMRI nude mice were divided into two cohorts: In cohort 1 (filled circles), 15 mice were inoculated subcutaneously with 5×10⁶ NCI-N87 gastric cancer cells into the right flank on day 0. Tumor volume was measured 2×/week by caliper. In cohort 2 (filled squares), 20 mice were inoculated subcutaneously with 5×10⁶ NCI-N87 cells on day-62. When tumor volumes reached a mean of 150 mm³, the animals were treated with 20 mg/kg of an αHER2 antibody amatoxin conjugate (XXb) comprising an αHER2-LALA-D265C antibody. 28 days after treatment all mice achieved complete tumor remission (tumor volume, TV<10 mm³). 27 days later (=day 0) the mice were re-inoculated with 5×10⁶ NCI-N87 into their right flank. Tumor growth was followed by caliper measurements 2×/week. In addition to the anti-tumor effect in heterogenous tumors, mice that were re-challenged with the same tumor cell line after complete tumor remission achieved by ATAC treatment did develop tumors at a significant lower rate than wildtype mice upon first inoculation, indicating NK cell-mediated tumor cell killing in these mice.

FIG. 10. Synergistic action of pemprolizumab and anti-CD19-Amatoxin conjugate treatment in vivo in a Raji xenograft mouse model system. Results of anti-tumoral studies using anti-PD-1 antibody pembrolizumab (20 mg/kg, i.v., days 0, 3, 6, 8, 10), or anti-CD19-amatoxin conjugate (anti-CD19 ATAC, 0.1 mg/kg or 0.3 mg/kg; single dose i.v. day 0), or a combination of pembrolizumab (20 mg/kg) and anti-CD19 ATAC (0.1 mg/kg or 0.3 mg/kg). Tumor volume was assessed at various time points after tumor cell inoculation. Error bars represent the SEM.

FIG. 11. Survival plot of mice depicting the synergistic effect of a combination treatment of anti-CTLA4 treatment and anti-CD19-amatoxin conjugate in a Raji xenograft mouse model. Anti-CD19 ATAC was administered at a dose of 0.1 mg/kg or 0.3 mg/kg single dose on day 0, ipilimumab was administered at a dose of 4 mg/kg on days 0, 3, 6, 8, and 10. Combination treatment was done using anti-CD19 ATAC at a dose of 0.1 mg/kg, or 0.3 mg/kg (both single dose) on day 0, and ipilimumab at a dose of 4 mg/kg on days 0, 3, 6, 8, 10. Experimental details are provided in Example 6.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure and avoid lengthy repetitions.

According to a first aspect of the present invention, a pharmaceutical composition is provided comprising
(a) at least one immune checkpoint inhibitor and
(b) at least one conjugate, wherein said conjugate is comprising
   (i) a target binding moiety,
   (ii) at least one amatoxin, and
   (iii) optionally at least one linker connecting said target binding moiety with said at least one amatoxin.

According to a second aspect of the present invention, a composition for use in the treatment of cancer is provided, said composition comprising
(a) at least one immune checkpoint inhibitor and
(b) at least one conjugate, wherein said conjugate is comprising
   (i) a target binding moiety,
   (ii) at least one amatoxin, and
   (iii) optionally at least one linker connecting said target binding moiety with said at least one amatoxin.

According to a third aspect of the present invention, a composition for use in the treatment of a chronic infectious disease is provided, said composition comprising
(a) at least one immune checkpoint inhibitor and
(b) at least one conjugate, wherein said conjugate is comprising
   (i) a target binding moiety,
   (ii) at least one amatoxin, and
   (iii) optionally at least one linker connecting said target binding moiety with said at least one amatoxin.

Immune checkpoints, also called immune checkpoint receptors, control T-cell activation and thus prevent overshooting inflammation and autoimmune diseases, but also suppress anti-tumor immune responses.

In the context of the present invention, the term “immune checkpoint inhibitor” or simply “checkpoint inhibitor” or “ICI” refers to any agent or compound that, cither directly or indirectly, decreases the level of or inhibits the function of an immune checkpoint receptor protein or molecule found on the surface of an immune cell (for example, a T cell), or to any agent or compound that, cither directly or indirectly, decreases the level of or inhibits the function of a ligand that binds to said immune checkpoint receptor protein or molecule, either as a soluble compound or on the surface of an immune cell-inhibitory cell. Such an inhibitory cell can be, for example, a cancer cell, a regulatory T cell, a tolerogenic antigen presenting cell, a myeloid-derived suppressor cells, a tumor-associated macrophage, or a cancer-associated fibroblast. Said ligand is typically capable of binding the immune checkpoint receptor protein or molecule on the immune cell. A non-limiting example of an immune checkpoint receptor protein-ligand pair is PD-1, PD-L1. PD-1 is an immune checkpoint receptor protein found on T-cells. PD-L1, which can be over-expressed by cancer cells, binds to PD-1 and helps the cancer cells to evade the host immune system attack. Accordingly, an immune checkpoint inhibitor prevents the PD-1/PD-L1 interaction by either blocking the PD-1 on the T cell (i.e. acts as a PD-I inhibitor) or the PD-L1 on the cancer cell (i.e., acts as a PD-L1 inhibitor), thereby maintaining or restoring anti-tumor T-cell activity or blocking inhibitory cancer cell activity.

Thus, immune checkpoint inhibitors are antagonists of an immune inhibitory receptor, such PD-1, which inhibit, in this case, the PD-1 or PD-L1 in the PD-1/PD-L1 pathway. Examples of PD-1 or PD-L1 inhibitors include, without limitation, humanized or human antibodies antagonizing or blocking human PD-1 function such as pembrolizumab, pidilizumab, cemiplimab, JTX-4014, spartalizumab, sintilimab (IBI308), dostarlimab (TSR-042, WBP-285), INCMGA00012 (MGA012), AMP-224, PD1-1, PD1-2, PD1-3, PD1-4, PD1-5, BCD-100, AGEN-2034, Toripalimab (TAB001, JS001), or AMP-514 (MEDI0680), as well as fully human antibodies such as the PD-1 blocking nivolumab or blocking PD-L1 such as avelumab, durvalumab, Cosibelimab (CK-301), WBP-3155 (CS1001) and atezolizumab or the recombinant anti-PD-L1 probody CX-072.

Pembrolizumab (formerly also known as lambrolizumab; trade name Keytruda; also known as MK-3475) disclosed e.g. in Hamid, O. et al. (2013) New England Journal of Medicine 369 (2): 134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WO2009/114335. It is approved by the FDA for the treatment of patients suffering from unresectable or metastatic melanoma and patients with metastatic NSCLC.

Nivolumab (CAS Registry Number: 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WO2006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgG1k monoclonal antibody that binds to PD-1. Pidilizumab is e.g. disclosed in WO2009/101611.

PD1-1 to PD1-5 refer to anti-PD-1 antibodies as disclosed in WO2018/220169.

Ipilumumab (CAS Registry Number: 477202-00-9, which may also be referred to as JODI, or MDX010. MDX-101) is a human IgG1 antibody that binds Cytotoxic T-lymphocyte antigen-4 (CTLA4). CTLA-4 is an inhibitory molecule that competes with the stimulatory CD28 for binding to B7 on antigen presenting cells. CTLA-4 and CD28 are both presented on the surface of T-cells. Ipilimumab is a human IgG1 that binds CTLA-4, preventing the inhibition of T-cell mediated immune responses to tumors. Ipilimumab is e.g. disclosed in WO 01/14424 as antibody “10D1”.

The INNs as used herein are meant to also encompass all biosimilar antibodies of the corresponding originator antibody as disclosed herein, including but not limited to those biosimilar antibodies authorized under 42 USC § 262 subsection (k) in the US and equivalent regulations in other jurisdictions.

Immune checkpoint receptors or molecules include, without limitation, e.g., PD-1, CTLA-4, LAG-3, TIM-3, TIGIT, VISTA, OX40, GITR, ICOS, CD276 (B7-H3), B7-H4 (VTCN1), IDO, KIR, CD122, CD137, CD94/NKG2A, CD80, CD86, Galectin-3, LSECtin, CD112, Ceacam-1, Gal-9, PtdSer, HMGB1, HVEM, CD155 and BTLA (CD272).

An immune checkpoint inhibitor according to the present invention may e.g. be a small molecule (organic) compound or a large molecule such as a peptide or a nucleic acid. For example, small molecule immune checkpoint inhibitors according to the invention include CA-170, including its precursor AUNP-12, as disclosed in WO15033301 A1; or e.g. BMS-8 (CAS number 1675201-90-7). In at least one embodiment of the present invention, an immune checkpoint inhibitor is an antibody, or an antigen binding fragement thereof, or an antigen binding derivative thereof. In a preferred embodiment, the immune checkpoint inhibitor is a monoclonal antibody, or an antigen binding fragment thereof, or an antigen binding derivative thereof.

In the context of the present invention the term “amatoxin” includes all cyclic peptides composed of 8 amino acids as isolated from the genus Amanita and described in Wieland, T. and Faulstich H. (Wieland T, Faulstich H., CRC Crit Rev Biochem. 5 (1978) 185-260), further all chemical derivatives thereof; further all semisynthetic analogs thereof; further all synthetic analogs thereof built from building blocks according to the master structure of the natural compounds (cyclic, 8 amino acids), further all synthetic or semisynthetic analogs containing non-hydroxylated amino acids instead of the hydroxylated amino acids, further all synthetic or semisynthetic analogs, in which the sulfoxide moiety is replaced by a sulfone, thioether, or by atoms different from sulfur, e.g., a carbon atom as in a carbanalog of amanitin.

As used herein, a “derivative” of a compound refers to a species having a chemical structure that is similar to the compound, yet containing at least one chemical group not present in the compound and/or deficient of at least one chemical group that is present in the compound. The compound to which the derivative is compared is known as the “parent” compound. Typically, a “derivative” may be produced from the parent compound in one or more chemical reaction steps.

As used herein, an “analogue” of a compound is structurally related but not identical to the compound and exhibits at least one activity of the compound. The compound to which the analogue is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analogue exhibits such an activity to the same extent as the parent compound. A compound is regarded as an analogue within the context of the present application, if it exhibits the relevant activity to a degree of at least 1% (more preferably at least 5%, more preferably at least 10%, more preferably at least 20%, more preferably at least 30%. more preferably at least 40%, and more preferably at least 50%) of the activity of the parent compound. Thus, an “analogue of an amatoxin”, as it is used herein, refers to a compound that is structurally related to any one of α-amanitin, β-amanitin, γ-amanitin, ε-amanitin, amanin, amaninamide, amanullin, and amanullinic acid and that exhibits at least 1% (more preferably at least 5%, more preferably at least 10%, more preferably at least 20%, more preferably at least 30%, more preferably at least 40%, and more preferably at least 50%) of the inhibitory activity against mammalian RNA polymerase II as compared to at least one of α-amanitin, β-amanitin, γ-amanitin, ε-amanitin, amanin, amaninamide, amanullin, and amanullinic acid. An “analogue of an amatoxin” suitable for use in the present invention may even exhibit a greater inhibitory activity against mammalian RNA polymerase II than any one of α-amanitin, β-amanitin, γ-amanitin, ε-amanitin, amanin, amaninamide, amanullin, or amanullinic acid. The inhibitory activity might be measured by determining the concentration at which 50% inhibition occurs (IC₅₀ value). The inhibitory activity against mammalian RNA polymerase II can be determined indirectly by measuring the inhibitory activity on cell proliferation.

A “semisynthetic analogue” refers to an analogue that has been obtained by chemical synthesis using compounds from natural sources (e.g. plant materials, bacterial cultures, fungal cultures or cell cultures) as starting material. Typically, a “semisynthetic analogue” of the present invention has been synthesized starting from a compound isolated from a mushroom of the Amanitaceae family. In contrast, a “synthetic analogue” refers to an analogue synthesized by so-called total synthesis from small (typically petrochemical) building blocks. Usually, this total synthesis is carried out without the aid of biological processes.

According to some embodiments of the present invention, the amatoxin of said conjugate can be selected from the group consisting of α-amanitin, β-amanitin, γ-amanitin, ε-amanitin, amanin, amaninamide, amanullin, amanullinic acid, and analogues, derivatives and salts thereof.

Functionally, amatoxins are defined as peptides or depsipeptides that inhibit mammalian RNA polymerase II. Preferred amatoxins are those with a functional group (e.g. a carboxylic group, an amino group, a hydroxy group, a thiol or a thiol-capturing group) that can be reacted with linker molecules or target-binding moieties as defined below.

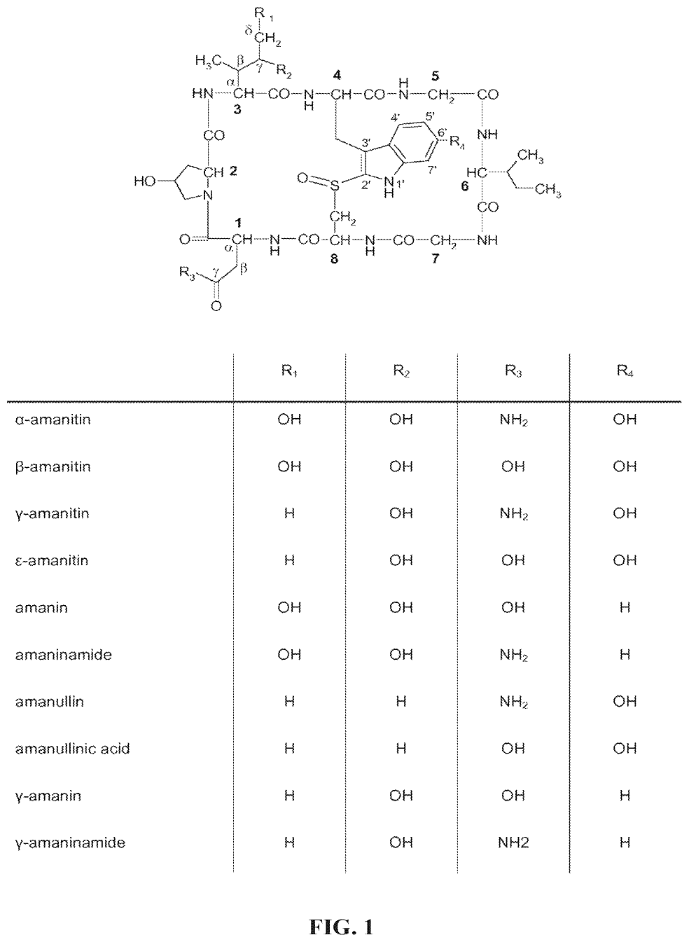

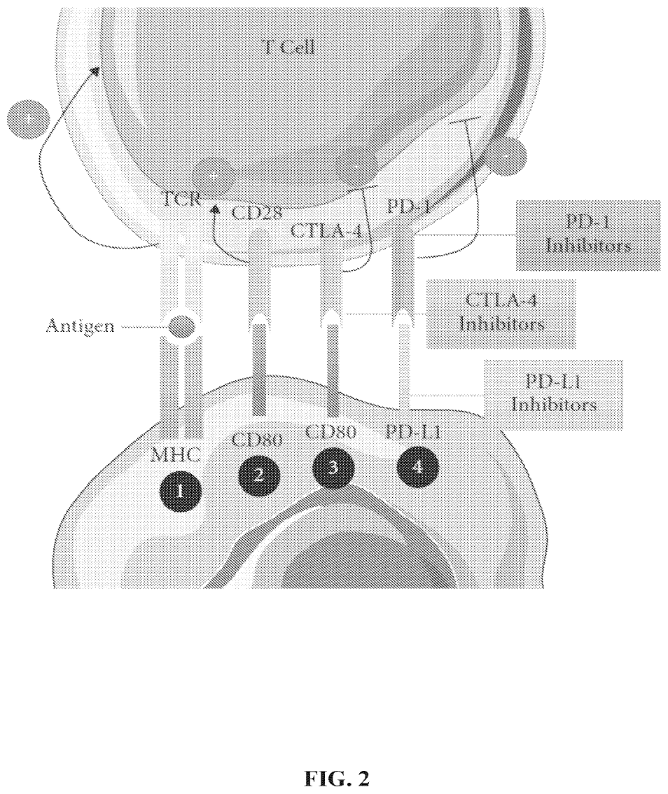

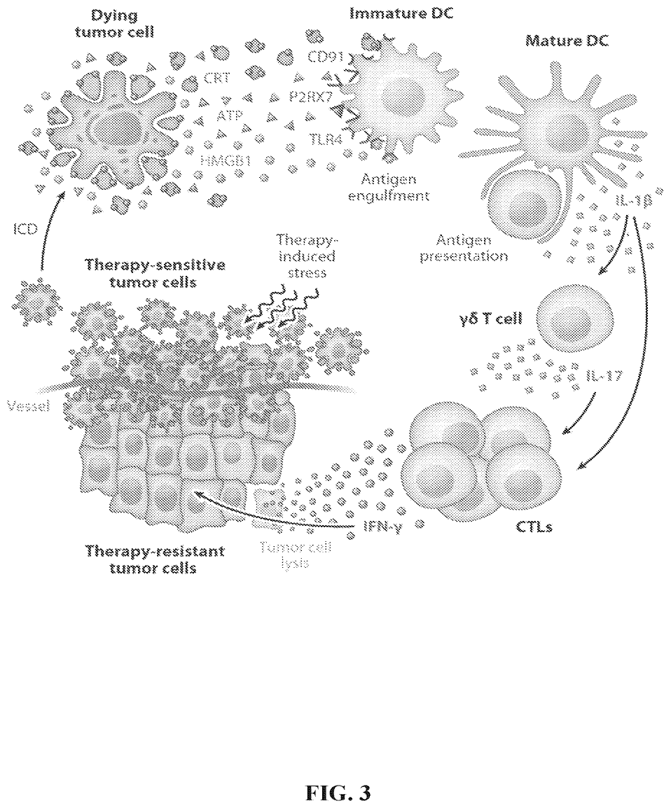

In the context of the present invention, the term “amanitins” particularly refers to bicyclic structure that are based on an aspartic acid or asparagine residue in position 1, a proline residue, particularly a hydroxyproline residue in position 2, an isoleucine, hydroxyisoleucine or dihydroxyisoleucine in position 3, a tryptophan or hydroxytryptophan residue in position 4, glycine residues in positions 5 and 7, an isoleucine residue in position 6, and a cysteine residue in position 8, particularly a derivative of cysteine that is oxidized to a sulfoxide or sulfone derivative (for the numbering and representative examples of amanitins, see FIG. 1), and furthermore includes all chemical derivatives thereof; further all semisynthetic analogues thereof; further all synthetic analogues thereof built from building blocks according to the master structure of the natural compounds (cyclic, 8 amino acids), further all synthetic or semisynthetic analogues containing non-hydroxylated amino acids instead of the hydroxylated amino acids, further all synthetic or semisynthetic analogues, in each case wherein any such derivative or analogue is functionally active by inhibiting mammalian RNA polymerase II.

The term “target-binding moiety”, as used herein, refers to any molecule or part of a molecule that can specifically bind to a target molecule or target epitope. Preferred target-binding moieties in the context of the present application are (i) antibodies or antigen-binding fragments thereof; (ii) antibody-like proteins; and (iii) nucleic acid aptamers. “Target-binding moieties” suitable for use in the present invention typically have a molecular mass of 40 000 Da (40 kDa) or more.

A “linker” in the context of the present application refers to a molecule that increases the distance between two components, e.g. to alleviate steric interference between the target binding moiety and the amatoxin, which may otherwise decrease the ability of the amatoxin to interact with RNA polymerase II. The linker may serve another purpose as it may facilitate the release of the amatoxin specifically in the cell being targeted by the target binding moiety. It is preferred that the linker and preferably the bond between the linker and the amatoxin on one side and the bond between the linker and the target binding moiety or antibody on the other side is stable under the physiological conditions outside the cell, e.g. the blood, while it can be cleaved inside the cell, in particular inside the target cell, e.g. cancer cell. To provide this selective stability, the linker may comprise functionalities that are preferably pH-sensitive or protease sensitive. Altematively, the bond linking the linker to the target binding moiety may provide the selective stability. Preferably a linker has a length of at least 1, preferably of 1-30 atoms length (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 atoms), wherein one side of the linker has been reacted with the amatoxin and, the other side with a target-binding moiety. In the context of the present invention, a linker preferably is a C₁₋₃₀-alkyl, C₁₋₃₀-heteroalkyl, C₂₋₃₀-alkenyl, C₂₋₃₀-heteroalkenyl, C₂₋₃₀-alkynyl, C₂₋₃₀-heteroalkynyl, cycloalkyl, heterocycloalkyl, aryl, heteroaryl, aralkyl, or a heteroaralkyl group, optionally substituted. The linker may contain one or more structural elements such as amide, ester, ether, thioether, disulfide, hydrocarbon moieties and the like. The linker may also contain combinations of two or more of these structural elements. Each one of these structural elements may be present in the linker more than once, e.g. twice, three times, four times, five times, or six times. In some embodiments the linker may comprise a disulfide bond. It is understood that the linker has to be attached either in a single step or in two or more subsequent steps to the amatoxin and the target binding moiety. To that end the linker to be will carry two groups, preferably at a proximal and distal end, which can (i) form a covalent bond to a group, preferably an activated group on an amatoxin or the target binding-peptide or (ii) which is or can be activated to form a covalent bond with a group on an amatoxin. Accordingly, if the linker is present, it is preferred that chemical groups are at the distal and proximal end of the linker, which are the result of such a coupling reaction, e.g. an ester, an ether, a urethane, a peptide bond etc. The presence of a “linker” is optional, i.e. the toxin may be directly linked to a residue of the target-binding moiety in some embodiments of the target-binding moiety toxin conjugate.

In a preferred embodiment of said composition of the first, second and third aspect, said immune checkpoint inhibitor and/or the target binding moiety of said conjugate is selected from the group consisting of
(i) an antibody, preferably a monoclonal antibody,
(ii) an antigen-binding fragment thereof, preferably a variable domain (Fv), a Fab fragment or an F(ab)₂ fragment,
(iii) an antigen-binding derivative thereof, preferably a single-chain Fv (scFv), and
(iv) an antibody-like protein.

Said antibody, or antigen-binding fragment thereof, or antigen-binding derivative thereof, can be a murine, a chimeric, a humanized or a human antibody, or antigen-binding fragment, or antigen-binding derivative thereof, respectively.

As used herein, the term “antibody” shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or V_{H}) and a heavy chain constant region (abbreviated as CH or C_{H}). The heavy chain constant region is comprised of three domains, namely CH1, CH2 and CH3. Each light chain contains a light chain variable region (abbreviated as VL or VI.) and a light chain constant region (abbreviated as CL or C_{L}). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

The term “antigen binding portion” of the (monoclonal) antibody refers to one or more fragments of an antibody which retain the ability to specifically bind to the CD20 antigen in its native form. Examples of antigen binding portions of the antibody include a Fab fragment, a monovalent fragment consisting of the VL, VH, CL and CH1 domains, an F(ab′)₂ fragment, a bivalent fragment comprising two Fab fragments linked by a disulfid bridge at the hinge region, an Fd fragment consisting of the VH and CH1 domain, an Fv fragment consisting of the VL and VH domains of a single arm of an antibody, and a dAb fragment which consists of a VH domain and an isolated complementarity determining region (CDR). The antibody, or antibody fragment or antibody derivative thereof, according to the present invention can be a monoclonal antibody. The antibody can be of the IgA, IgD, IgE, IgG or IgM isotype.

The term “monoclonal antibody” (“mAb”) as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity. Preferably, such antibody is selected from the group consisting of IgG, IgD, IgE, IgA and/or IgM, or a fragment or derivative thereof, more preferably such antibody is an IgG type antibody or fragment or derivative thereof.

As used herein, the term “fragment” shall refer to fragments of such antibody retaining target binding capacities, e.g., a CDR (complementarity determining region), a hypervariable region, a variable domain (Fv), an IgG heavy chain (consisting of VH, CH1, hinge, CH2 and CH3 regions), an IgG light chain (consisting of VL and CL regions), and/or a Fab and/or F(ab)₂.

As used herein, the term “antigen-binding derivative” or “derivative” shall refer to protein constructs being structurally different from, but still having some structural relationship to, the common antibody concept, e.g. a protein composed of a peptide scaffold and at least one of the CDRs of the original antibody it is derived from. Examples include e.g. scFv, Fab and/or F(ab)₂, as well as bi-, tri-or higher specific antibody constructs. All these items are explained below.

Other antibody derivatives known to the skilled person are Diabodies, Camelid Antibodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, other scaffold protein formats comprising CDRs, and antibody conjugates (e.g., antibody, or fragments or derivatives thereof, linked to a drug, a toxin, a cytokine, an aptamer, a nucleic acid such as a desoxyribonucleic acid (DNA) or ribonucleic acid (RNA), a therapeutic polypeptide, a radioisotope or a label). Said scaffold protein formats may comprise, for example, antibody-like proteins such as ankyrin and affilin proteins and others.

As used herein, the term “antibody-like protein” refers to a protein that has been engineered (e.g. by mutagenesis of Ig loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affibodies, anticalins, and designed ankyrin repeat proteins (Binz et al., 2005). Antibody-like proteins can be derived from large libraries of mutants, e.g. by panning from large phage display libraries, and can be isolated in analogy to regular antibodies. Also, antibody-like binding proteins can be obtained by combinatorial mutagenesis of surface-exposed residues in globular proteins.

As used herein, the term “Fab” relates to an IgG fragment comprising the antigen binding region, said fragment being composed of one constant and one variable domain from each heavy and light chain of the antibody.

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually comprising serine(S) and/or glycine (G) residues. This chimeric molecule retains the specificity of the original immunoglobulin, despite removal of the constant regions and the introduction of a linker peptide.

Modified antibody formats are for example bi-or trispecific antibody constructs, antibody-based fusion proteins, immunoconjugates and the like.

IgG, scFv, Fab and/or F(ab)₂ are antibody formats which are well known to the skilled person. Related enabling techniques are available from respective textbooks.

According to preferred embodiments of the present invention, said antibody, or antigen-binding fragment thereof or antigen-binding derivative thereof, is a murine, a chimeric, a humanized or a human antibody, or antigen-binding fragment or antigen-binding derivative thereof, respectively.

Monoclonal antibodies (mAb) derived from mouse may cause unwanted immunological side-effects due to the fact that they contain a protein from another species which may elicit antibodies. In order to overcome this problem, antibody humanization and maturation methods have been designed to generate antibody molecules with minimal immunogenicity when applied to humans, while ideally still retaining specificity and affinity of the non-human parental antibody (for review see Almagro and Fransson 2008). Using these methods, e.g., the framework regions of a mouse mAb are replaced by corresponding human framework regions (so-called CDR grafting). WO200907861 discloses the generation of humanized forms of mouse antibodies by linking the CDR regions of non-human antibodies to human constant regions by recombinant DNA technology. U.S. Pat. No. 6,548,640 by Medical Research Council describes CDR grafting techniques, and U.S. Pat. No. 5,859,205 by Celltech describes the production of humanised antibodies.

As used herein, the term “humanized antibody” relates to an antibody, a fragement or a derivative thereof, in which at least a portion of the constant regions and/or the framework regions, and optionally a portion of CDR regions, of the antibody is derived from or adjusted to human immunoglobulin sequences.

The antibodies, the antibody fragments or antibody derivatives thereof, disclosed herein can comprise humanized sequences, in particular of the preferred VH- and VL-based antigen-binding region which maintain appropriate ligand affinity. The amino acid sequence modifications to obtain said humanized sequences may occur in the CDR regions and/or in the framework regions of the original antibody and/or in antibody constant region sequences.

Said antibody, or antibody fragment or antibody derivative thereof, can be glycosylated. The glycan can be an N-linked oligosaccharide chain at asparagin 297 of the heavy chain.

The antibodies or fragments or derivatives of the present invention may be produced by transfection of a host cell with an expression vector comprising the coding sequence for the antibody according to the invention. The expression vector or recombinant plasmid is produced by placing the coding antibody sequences under control of suitable regulatory genetic elements, including promoter and enhancer sequences like, e.g., a CMV promoter. Heavy and light chain sequences might be expressed from individual expression vectors which are co-transfected, or from dual expression vectors. Said transfection may be a transient transfection or a stabile transfection. The transfected cells are subsequently cultivated to produce the transfected antibody construct. When stabile transfection is performed, then stable clones secreting antibodies with properly associated heavy and light chains are selected by screening with an appropriate assay, such as, e.g., ELISA, subcloned, and propagated for future production.

In some embodiments of the composition according to the present invention, said immune checkpoint inhibitor binds to an immune checkpoint receptor selected from the group consisting of PD-1, CTLA-4, LAG-3, TIGIT, TIM-3, VISTA, BTLA (CD272), OX40 (CD134), B7-H4 (VTCN1), CD96, CD278 (ICOS), CD94/NKG2A and CD160, or to a ligand of an immune checkpoint receptor selected from the group consisting of PD-L1, PD-L2, CD80,CD86, Galectin-3, LSECtin, CD112, Ceacam-1, Gal-9, PtdSer, HMGB1, HVEM, CD155, OX40L, CD275 (ICOSLG).

In some embodiments, the composition according to the present invention comprises an immune checkpoint inhibitor, wherein said immune checkpoint inhibitor is an antibody selected from the group consisting of nivolumab, pembrolizumab, pidilizumab, cemiplimab, JTX-4014, spartalizumab, sintilimab (IBI308), dostarlimab (TSR-042, WBP-285), INCMGA00012 (MGA012), AMP-224, PD1-1, PD1-2, PD1-3, PD1-4, PD1-5, BCD-100, AGEN-2034, Toripalimab (TAB001, JS001), or AMP-514 MEDI0680 nivolumab, avelumab, durvalumab, cosibelimab (CK-301), WBP-3155 (CS1001), atezolizumab or CX-072, or an antigen-binding fragment thereof, or an antigen-binding derivative thereof. Preferably, preferably wherein the antibody is one of avelumab, nivolumab, ipilimumab, pembrolizumab, or an antigen-binding fragment thereof, or an antigen-binding derivative thereof.

In some embodiments of the invention, the composition according to the present invention as disclosed herein comprises a combination of two or more immune checkpoint inhibitors, e.g. two, three, four, five, six immune checkpoint inhibitors as disclosed herein, preferably the composition comprises a combination of two immune checkpoint inhibitors. For example, it is preferred that the composition according to the invention as disclosed herein comprises two or more immune checkpoint inhibitors that target different immune checkpoints, e.g. CTLA-4 and PD-1/PD-L1, PD-1/PD-L1 and TIGIT, PD-1/PD-L1 and OX40, PD-1/PD-L1 and VISTA, CTLA4 and TIGIT, CTLA4 and OX40.

Accordingly, compositions according to the invention may e.g. comprise one of the following combinations of immune checkpoint inhibtors:

### CTLA4-PD-1/PD-L1

Ipilimumab in combination with one of nivolumab, avelumab, pembrolizumab, pidilizumab, PD1-1, PD1-2, PD1-3, PD1-4, PD1-5, durvalumab, atezolizumab

### PD-1/PD-L1 and TIGIT

Tiragolumab in combination with one of nivolumab, avelumab, pembrolizumab, pidilizumab, PD1-1, PD1-2, PD1-3, PD1-4, PD1-5, durvalumab, atezolizumab; or, BMS986207 in combination with one of nivolumab, avelumab, pembrolizumab, pidilizumab, PD1-1, PD1-2, PD1-3, PD1-4, PD1-5, durvalumab, atezolizumab;

### PD-1/PD-L1 and OX40

BMS986178 in combination with one of nivolumab, avelumab, pembrolizumab, pidilizumab, PD1-1, PD1-2, PD1-3, PD1-4, PD1-5, durvalumab, atezolizumab

### PD-1/PD-L1 and VISTA

CI-8993 in combination with one of nivolumab, avelumab, pembrolizumab, pidilizumab, PD1-1, PD1-2, PD1-3, PD1-4, PD1-5, durvalumab, atezolizumab

### PD-1/PD-L1 and OX40

MEDI0562 in combination with one of nivolumab, avelumab, pembrolizumab, pidilizumab, PD1-1, PD1-2, PD1-3, PD1-4, PD1-5, durvalumab, atezolizumab, or PF04518600 in combination with one of nivolumab, avelumab, pembrolizumab, pidilizumab, PD1-1, PD1-2, PD1-3, PD1-4, PD1-5, durvalumab, atezolizumab

### TIM-3 and PD-1/PD-L1

MBG453 in combination with one of of nivolumab, avelumab, pembrolizumab, pidilizumab, PD1-1, PD1-2, PD1-3, PD1-4, PD1-5, durvalumab, atezolizumab

### CTLA4 and TIGIT

Ipilimumab in combination with one of tiragolumab, or BMS986207.

### CTLA4 and OX40

Ipilimumab in combination with one of MEDI0562, or PF04518600.

In some embodiments, the composition according to the present invention comprises a target binding moiety, wherein said target binding moiety of said conjugate binds to a target molecule on the cell surface of cancer cells wherein the target molecule is one of PSMA, CD19, CD37, CD269, sialyl Lewis^{a}, HER-2/neu, epithelial cell adhesion molecule (EpCAM). The target molecules PSMA, CD19, CD37, CD269, sialyl Lewis^{a}, HER-2/neu, epithelial cell adhesion molecule (EpCAM) as disclosed above refer to the following proteins or cell surface antigens: “PSMA” as used herein refers to prostate specific membrane antigen, which is also known as glutamate carboxypeptidase II (GCPII), N-acetyl-L-aspartyl-L-glutamate peptidase I (NAALADase I) or N-acetyl-aspartylglutamate (NAAG) peptidase, is an enzyme that is encoded by the human folate hydrolase (FOLH1) gene. PSMA is a membrane-bound cell surface peptidase that plays different physiological roles and is expressed in various tissues such as the prostate, kidney, small intestine, central and peripheral nervous system. It is highly expressed by malignant prostate epithelial cells and vascular endothelial cells of numerous solid tumor malignancies, including glioblastomas, breast and bladder cancers.

“CD19” as disclosed above refers to B-lymphocyte antigen CD19, also known as CD19 molecule (Cluster of Differentiation 19), B-Lymphocyte Surface Antigen B4, T-Cell Surface Antigen Leu-12 and CVID3 which is a transmembrane protein that in humans is encoded by the gene CD19. CD19 is a biomarker for B lymphocyte development, lymphoma diagnosis and can be utilized as a target for leukemia immunotherapies.

The term “CD37” as disclosed above refers to a protein that in humans is encoded by the CD37 gene and is a member of the “tetraspanin” superfamily or transmembrane 4 superfamily. Tetraspanins are characterized by the presence of four conserved transmembrane domains that are considered as “molecular facilitators” of signaling transduction, involved in a wide range of biological processes including cell growth, survival, adhesion, cell-cell communication, and trafficking, intercellular communication via exosomes, tumorigenesis, metastasis, and regulation of immune responses. Tetraspanin members have also been described to have functional roles in a wide array of cellular processes, including cell motility, development and differentiation, activation, proliferation, migration and tumor invasion (Hemler 2001; Xu-Monette et al. 2016). Increased CD37 expression was found in B cell malignancies (Zou et al. 2018). Most B-cell malignancies express CD37, including B-cell non-Hodgkin lymphoma (NHL) and B-cell chronic lymphocytic leukemia (B-CLL).

Target molecule CD269 as disclosed above refers to B-cell maturation antigen (BCMA or BCM), also known as tumor necrosis factor receptor superfamily member 17 (TNFRSF17), which is a protein that in humans is encoded by the TNFRSF17 gene. CD269 is implicated in leukemia, lymphomas, and multiple myeloma.

Sialyl Lewis^{a} (also referred to as CD15) as disclosed above refers to a tetrasaccharide composed of a sialic acid, fucose and an N-acetyllactosamine. Sialyl Lewis^{a} mediates phagocytosis and chemotaxis, found on neutrophils; expressed in patients with Hodgkin disease, as well as in some B-cell chronic lymphocytic leukemias, acute lymphoblastic leukemias, and most acute nonlymphocytic leukemias. Sialyl Lewis^{a} is present on almost all Reed-Sternberg cells, and can be used in immunohistochemistry to identify the presence of such cells in biopsies which is diagnostic of Hodgkin's lymphoma.

HER-2/neu as disclosed above refers to the receptor tyrosine-protein kinase erbB-2, also known as CD340 (cluster of differentiation 340), proto-oncogene Neu, Erbb2 (rodent), or ERBB2 (human), is a protein that in humans is encoded by the ERBB2 gene. ERBB is abbreviated from erythroblastic oncogene B, a gene isolated from avian genome. It is also frequently called HER2 (from human epidermal growth factor receptor 2) or HER2/neu. HER2 is a member of the human epidermal growth factor receptor (HER/EGFR/ERBB) family. Dimerization of the receptor results in the autophosphorylation of tyrosine residues within the cytoplasmic domain of the receptors and initiates a variety of signaling pathways leading to cell proliferation and tumorigenesis. Amplification or overexpression of HER2 occurs in approximately 15-30% of breast cancers and 10-30% of gastric/gastroesophageal cancers and serves as a prognostic and predictive biomarker.

EpCAM as disclosed above refers to “epithelial cell adhesion molecule” which is a transmembrane glycoprotein mediating Ca2+-independent homotypic cell-cell adhesion in epithelia. EpCAM is a glycosylated type 1 membrane protein having a molecular weight of 30-40 kD and in humans is endcoded by the EPCAM gene. The sequence of the EpCAM molecule predicts the presence of three potential N-linked glycosylation sites. It is composed of 314 amino acids. EpCAM consists of an extracellular domain (242 amino acids) with epidermal growth factor (EGF)- and thyroglobulin repeat-like domains, a single transmembrane domain (23 amino acids), and a short intracellular domain (26 amino acids). EpCAM is involved in cell signaling, migration, proliferation, and differentiation. In addition, EpCAM has oncogenic potential via its capacity to upregulate c-myc. e-fabp, and cyclins A & E. EpCAM may also be referred to as Ep-CAM, 17-1A, HEA125, MK-1, GA733-2, EGP-2, EGP34, KSA, TROP-1, ESA, or KS1/4. EpCAM is expressed exclusively in epithelia and epithelial-derived neoplasms and can be used as diagnostic marker for various cancers.

In the context of the present application the terms “target molecule” and “target epitope”, respectively, refers to an antigen and an epitope of an antigen, respectively, that is specifically bound by a target-binding moiety. Particularly the target molecule is a tumour-associated antigen, in particular an antigen or an epitope which is present on the surface of one or more tumour cell types in an increased concentration and/or in a different steric configuration as compared to the surface of non-tumour cells. Particularly, said antigen or epitope is present on the surface of one or more tumour cell types, but not on the surface of non-tumour cells. In particular embodiments, the target-binding moiety specifically binds to an epitope of an antigen selected from: PSMA, CD19, CD37, CD269, sialyl Lewis^{a}, HER-2/neu, epithelial cell adhesion molecule (EpCAM). In other embodiments, said antigen or epitope is preferentially expressed on cells involved in autoimmune diseases. In particular embodiments, the target-binding moiety specifically binds to an epitope of the IL-6 receptor (IL-6R).

In some embodiments, the composition according to the present invention as disclosed herein comprises a conjugate comprising a target binding moiety, wherein said target binding moiety of said conjugate is an antibody having an Fc region comprising at least one mutation selected from the group consisting of D265C, D265A, A118C, L234A, or L235A (according to the EU numbering system). The “EU index as in Kabat” or “EU numbering system” refers to the numbering of the human IgG1 EU antibody (see e.g. Edelman et al., 1969, Proc Natl Acad Sci USA 63:78-85, hereby entirely incorporated by reference).

In a preferred embodiment, the antibody representing the target-binding moiety of a conjugate according to the present invention as disclosed herein comprises an Fc region carrying a D265C mutation and said linker, if present, or said amatoxin is connected to said antibody via one or two of the D265C residues of said antibody, preferably via a disulfide linkage.

According to some embodiments of the present invention, said antibody representing the target-binding moiety of said conjugate has been genetically engineered to comprise a heavy chain 118Cys, a heavy chain 239Cys, or heavy chain 265Cys according to the EU numbering system, preferably a heavy chain 265Cys according to the EU numbering system, and wherein said linker, if present, or said amatoxin is connected to said antibody via said heavy chain 118Cys, or said heavy chain 239Cys, or heavy chain 265Cys residue, respectively.

According to preferred embodiments, the antibody representing the target-binding moiety of said conjugate according to the invention as disclosed herein comprises an Fc region which comprises a L234 mutation, a L235 mutation and a D265 mutation.

According to a more preferred embodiment, the antibody representing the target-binding moiety of said conjugate according to the invention as disclosed herein comprises an Fc region comprising a L234A, L235A and a D265C mutation (according to EU numbering system).

As used herein, the term “genetically engineered” or “genetic engineering” relates to the modification of the amino acid sequence or part thereof of a given or natural polypeptide or protein in the sense of nucleotide and/or amino acid substitution, insertion, deletion or reversion, or any combinations thereof, by gene technological methods such as e.g. site-directed mutagenesis as described in Biochem. J. (1986) 237, 1-7, or J Biol Chem. 2015 Jan. 30; 290 (5): 2577-2592.

As used herein, the term “amino acid substitution” relates to modifications of the amino acid sequence of the protein, wherein one or more amino acids are replaced with the same number of different amino acids, producing a protein which contains a different amino acid sequence than the original protein. A conservative amino acid substitution is understood to relate to a substitution which due to similar size, charge, polarity and/or conformation does not significantly affect the structure and function of the protein. Groups of conservative amino acids in that sense represent, e.g., the non-polar amino acids Gly, Ala, Val, Ile and Leu; the aromatic amino acids Phe, Trp and Tyr; the positively charged amino acids Lys, Arg and His; and the negatively charged amino acids Asp and Glu. Exemplary amino acid substitutions are presented in Table 1 below:

| Original residues | Examples of substitutions |
|---|---|
| Ala (A) | Val, Leu, Ile, Gly |
| Arg (R) | His, Lys |
| Asn (N) | Gln |
| Asp (D) | Glu |
| Cys (C) | Ser |
| Gln (Q) | Asn |
| Glu (E) | Asp |
| Gly (G) | Pro, Ala |
| His (H) | Lys, Arg |
| Ile (I) | Leu, Val, Met, Ala, Phe |
| Leu (L) | Ile, Val, Met, Ala, Phe |
| Lys (K) | Arg, His |
| Met (M) | Leu, Ile, Phe |
| Phe (F) | Leu, Val, Ile, Tyr, Trp, Met |
| Pro (P) | Ala, Gly |
| Ser (S) | Thr |
| Thr (T) | Ser |
| Trp (W) | Tyr, Phe |
| Tyr (Y) | Trp, Phe |
| Val (V) | Ile, Met, Leu, Phe, Ala |

According to some embodiments of the present invention, said linker, if present, or said amatoxin is connected to said antibody via any of the natural Cys residues of said antibody, preferably via a disulfide linkage.

Furthermore, the conjugate according to the present invention can have a cytotoxic activity of an IC₅₀ better than 10×10⁻⁹ M, 9×10⁻⁹ M, 8×10⁻⁹ M, 7×10⁻⁹ M, 6×10⁻⁹ M, 5×10⁻⁹ M, 4×10⁻⁹ M, 3×10⁻⁹ M, 2×10⁻⁹ M, preferably better than 10×10⁻¹⁰ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ m, and more preferably better than 10×10⁻¹¹ M, 9×10⁻¹¹ M, 8×10⁻¹¹ M, 7×10⁻¹¹ M, 6×10⁻¹¹ M, 5×10⁻¹¹ M, 4×10⁻¹¹ M, 3×10⁻¹¹ M, 2×10⁻¹¹ M, or 1×10⁻¹¹ M.

In some embodiments of the present invention, said conjugate as described comprises an amatoxin comprising (i) an amino acid 4 with a 6′-deoxy position and (ii) an amino acid 8 with an S-deoxy position.

In some embodiments of the present invention, the linker of said conjugate, if present, or said target binding moiety is connected to said amatoxin via (i) the γ C-atom of amatoxin amino acid 1, or (ii) the δ C-atom of amatoxin amino acid 3, or (iii) the 6′-C-atom of amatoxin amino acid 4.

According to preferred embodiments of the present invention, said conjugate in the composition as described comprises a linker.

Said linker can be a stable (non-cleavable) or a cleavable linker. The cleavable linker can be selected from the group consisting of an enzymatically cleavable linker, preferably a protease-cleavable linker, and a chemically cleavable linker, preferably a linker comprising a disulfide bridge.

A “cleavable linker” is understood as comprising at least one cleavage site. As used herein, the term “cleavage site” shall refer to a moiety that is susceptible to specific cleavage at a defined position under particular conditions. Said conditions are, e.g., specific enzymes or a reductive environment in specific body or cell compartments.

A “non-cleavable linker” is understood not to be subject to enzymatical cleavage by e.g. cathepsin B and is released from the conjugates of the invention during degradation (e.g., lysosomal degradation) of the antibody moiety of the conjugate of the invention inside the target cell.

According to some embodiments of the present invention, the cleavage site is an enzymatically cleavable moiety comprising two or more amino acids. Preferably, said enzymatically cleavable moiety comprises a valine-alanine (Val-Ala), valine-citrulline (Val-Cit), valine-lysine (Val-Lys), valine-arginine (Val-Arg) dipeptide, a phenylalanine-lysine-glycine-proline-leucin-glycine (Phe Lys Gly Pro Leu Gly) or alanine-alanine-proline-valine (Ala Ala Pro Val) peptide, or a β-glucuronide or β-galactoside. The enzymatically cleavable moiety may also be referred to as linker.

In particularly preferred embodiments, the enzymatically cleavable moiety according to the invention comprises a dipeptide selected from Phe-Lys, Val-Lys, Phe-Ala, Val-Ala, Phe-Cit and Val-Cit, particularly wherein the cleavable linker further comprises a p-aminobenzyl (PAB) spacer between the dipeptides and the amatoxin:

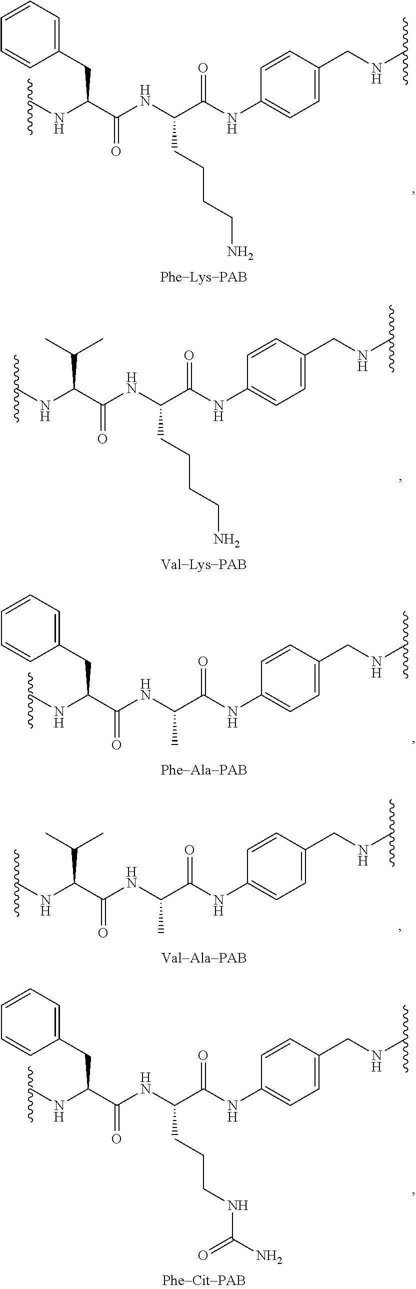 [SMILES: *NCc1ccc(NC(=O)[C@H](CCCCN)NC(=O)[C@H](Cc2ccccc2)N*)cc1 ; *NCc1ccc(NC(=O)[C@H](CCCCN)NC(=O)[C@@H](N*)C(C)C)cc1 ; *NCc1ccc(NC(=O)[C@H](C)NC(=O)[C@H](Cc2ccccc2)N*)cc1 ; *NCc1ccc(NC(=O)[C@H](C)NC(=O)[C@@H](N*)C(C)C)cc1 ; *NCc1ccc(NC(=O)[C@H](CCCNC(N)=O)NC(=O)[C@H](Cc2ccccc2)N*)cc1]

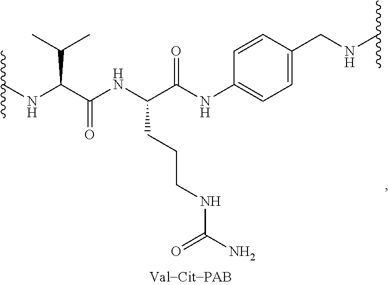 [SMILES: *NCc1ccc(NC(=O)[C@H](CCCNC(N)=O)NC(=O)[C@@H](N*)C(C)C)cc1]

Accordingly, the conjugates of the invention as disclosed herein can comprise an enzymatically cleavable moiety which comprises any one of the dipeptides-PAB moieties Phe-Lys-PAB, Val-LysPAB, Phe-Ala-PAB, Val-Ala-PAB, Phe-Cit-PAB, or Val-Cit-PAB as disclosed above. Preferably, the cleavable moiety of the conjugates of the invention comprises the dipeptide-PAB moiety Val-Ala-PAB.

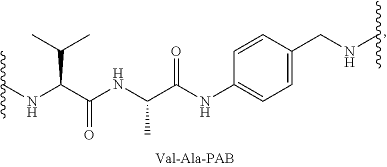 [SMILES: *NCc1ccc(NC(=O)[C@H](C)NC(=O)[C@@H](N*)C(C)C)cc1]

whereby the PAB moiety is linked to the amatoxin.

According to some embodiments, the cleavable moieties or linkers of the invention as disclosed above comprise a thiol reactive group, selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or a maleimide.

According to a preferred embodiment the thiol reactive group is a maleimide (maleimidyl moiety) as depicted below:

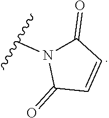 [SMILES: *N1C(=O)C=CC1=O]

According to a particularly preferred embodiment, the linker of the invention comprises the structure (i) prior to coupling, or (ii) following the coupling to an antibody as disclosed herein.

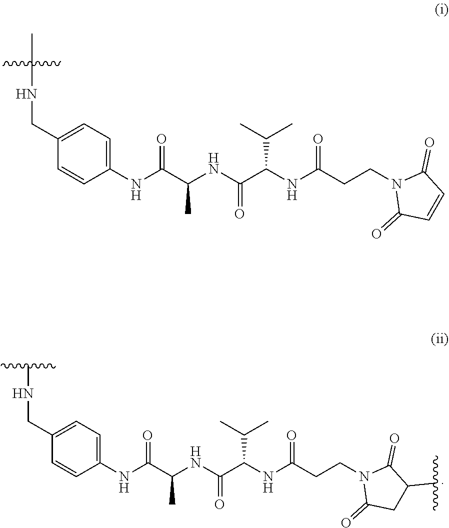 [SMILES: *NCc1ccc(NC(=O)[C@H](C)NC(=O)[C@@H](NC(=O)CCN2C(=O)C=CC2=O)C(C)C)cc1 ; *NCc1ccc(NC(=O)[C@H](C)NC(=O)[C@@H](NC(=O)CCN2C(=O)CC(*)C2=O)C(C)C)cc1]

According to some embodiments, said cleavage site can be cleavable by at least one protease selected from the group consisting of cysteine protease, metalloprotease, serine protease, threonine protease, and aspartic protease.

Cysteine proteases, also known as thiol proteases, are proteases that share a common catalytic mechanism that involves a nucleophilic cysteine thiol in a catalytic triad or dyad.

Metalloproteases are proteases whose catalytic mechanism involves a metal. Most metalloproteases require zinc, but some use cobalt. The metal ion is coordinated to the protein via three ligands. The ligands coordinating the metal ion can vary with histidine, glutamate, aspartate, lysine, and arginine. The fourth coordination position is taken up by a labile water molecule.

Serine proteases are enzymes that cleave peptide bonds in proteins; serine serves as the nucleophilic amino acid at the enzyme's active site. Serine proteases fall into two broad categories based on their structure: chymotrypsin-like (trypsin-like) or subtilisin-like.

Threonine proteases are a family of proteolytic enzymes harbouring a threonine (Thr) residue within the active site. The prototype members of this class of enzymes are the catalytic subunits of the proteasome, however, the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin.

In some embodiments of the present invention, the cleavable site is cleavable by at least one agent selected from the group consisting of Cathepsin A or B, matrix metalloproteinases (MMPs), elastases, β-glucuronidase and β-galactosidase, preferably Cathepsin B.

In some embodiments of the present invention, the cleavage site is a disulfide bond and specific cleavage is conducted by a reductive environment, e.g., an intracellular reductive environment, such as, e.g., acidic pH conditions.

According to some embodiments, the conjugate of the invention as disclosed herein comprises a non-cleavable linker. Non-cleavable linkers suitable for use according to the invention may e.g. include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include (CH₂)ₚ, (C═O)(CH₂)ₚ, and polyethyleneglycol (PEG; (CH₂CH₂O)ₚ), units, wherein p is an integer from 1-6, independently selected for each occasion.

In some embodiments, the non-cleavable linker of the invention comprises one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH2CH2O)p— group where p is an integer from 1-6, wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

For example, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene of the non-cleavable linker as disclosed herein may optionally be interrupted by one or more heteroatoms selected from O, S and N and may e.g. be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro. The definitions of the chemical groups as used herein shall have the meaning and be defined as provided in “Compendium of Chemical Terminology” (“Gold Book”) published by the International Union of Pure and Applied Chemistry (IUPAC) version 2.3.3, goldbook.iupac.org, ISBN: 0-9678550-9-8), the content of which is hereby incorporated by reference.

According to preferred embodiments, the non-cleavable linker of the conjugate of the invention comprises a —(CH₂)ₙ— unit, where n is an integer from, 2-12, e.g., 2-6, e.g. n is 1, 2, 3, 4, 5, or 6.

In a preferred embodiment, the non-cleavable linker of the conjugate of the invention comprises —(CH₂)ₙ— wherein n is 6 and the linker is represented by the formula:

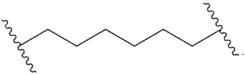 [SMILES: *CCCCCC*]

In some embodiments, the non-cleavable linkers of the invention as disclosed herein further comprise a thiol-reactive group. The thio-reactive group of said non-cleavable linkers as disclosed above may e.g. be selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or a maleimide.

According to a preferred embodiment, the thiol reactive group is a maleimide (maleimidyl moiety) as disclosed above. For example, the non-cleavable linker comprising said maleimide may e.g. have the following structure, whereby the wavy line at the linker terminus indicates the point of attachment to the amatoxin:

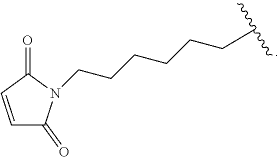 [SMILES: *CCCCCCN1C(=O)C=CC1=O]

Following conjugation to a reactive sulfhydryl on e.g. an antibody or target binding moiety as disclosed herein the meleimidyl moiety of e.g. cleavable or non-cleavable linker as disclosed herein comprise the structure:

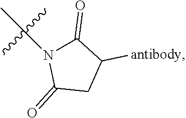

whereby the wavy line represents the attachment site of a cleavable or non-cleavable linker as disclosed herein.

According to preferred embodiments, conjugates of the invention comprising a cleavable or non-cleavable linker which further comprise a thiol-reactive group may be coupled to a naturally occurring sulfhydryl moiety in the antibody of the conjugate, or said cleavable or non-cleavable linker of the conjugates of the invention comprising a thiol-reactive group may be coupled to a sulfhydryl moiety which has been introduced into the antibody by genetic engineering as described in e.g. Nat Biotechnol. 2008 August; 26 (8): 925-32. Preferably, the cleavable or non-cleavable linker as disclosed herein which comprise a thio-reactive group are coupled to sulfhydryl moieties that have been introduced into the Fc region of the antibody of the conjugate of the invention by genetic engineering. Preferred positions within the Fc region of said antibody at which sulfhydryl moieties may be introduced comprise D265, or A118 (according to EU numbering), more preferably D265.

According to preferred embodiments of the present invention, the conjugate in said composition comprises any of the following compounds of formulas (I) to (XI), respectively, as linker-amatoxin moieties:

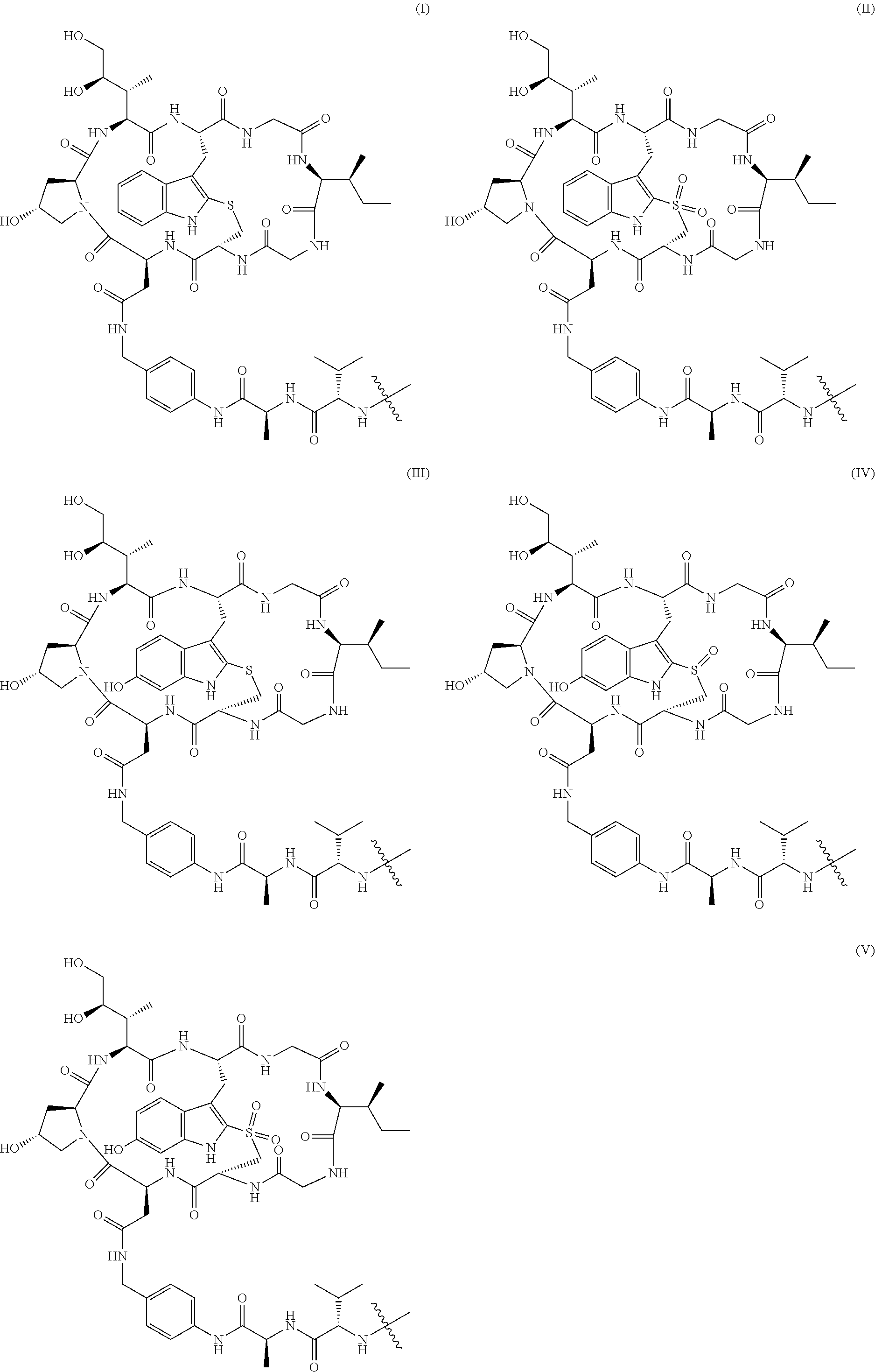

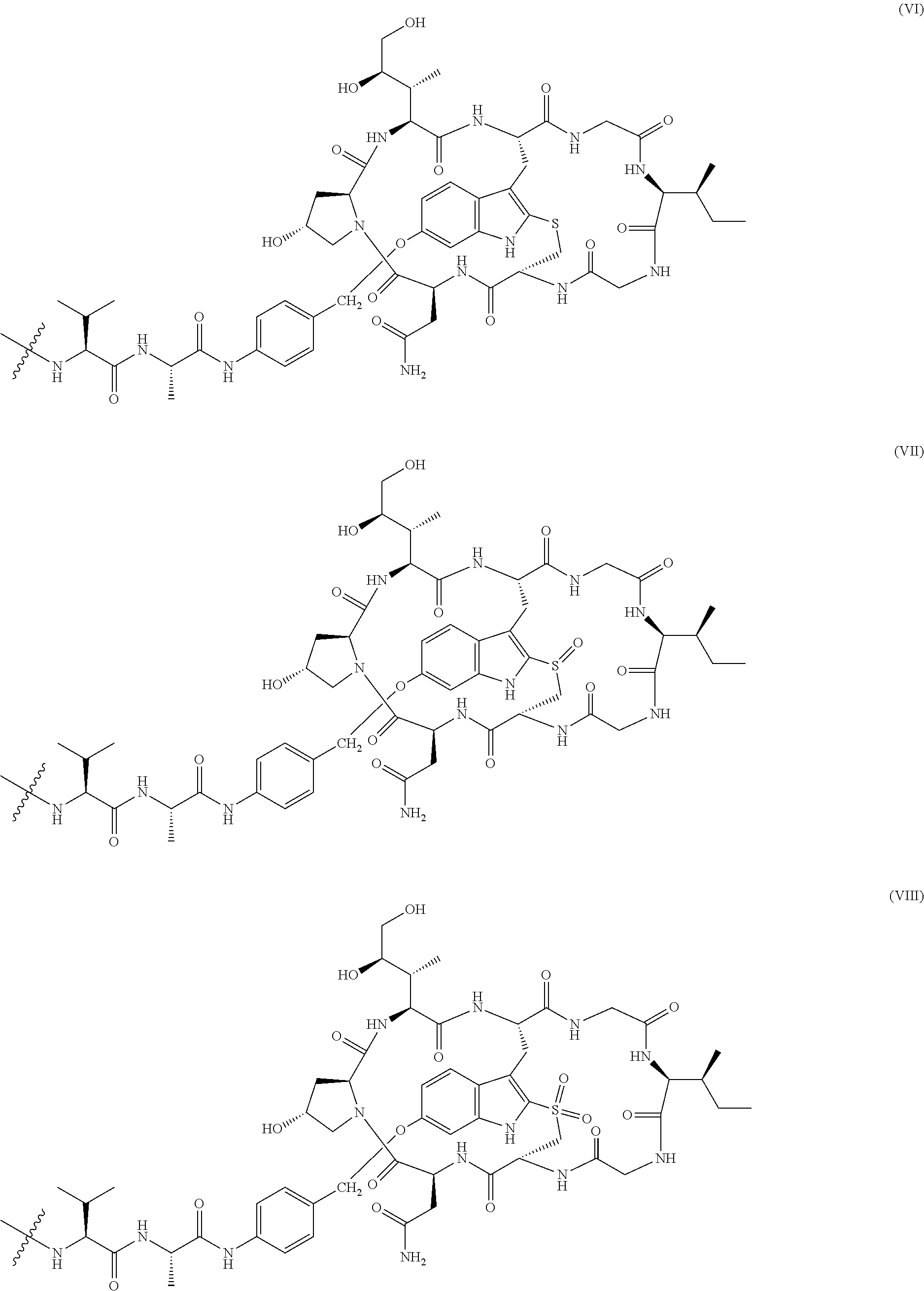

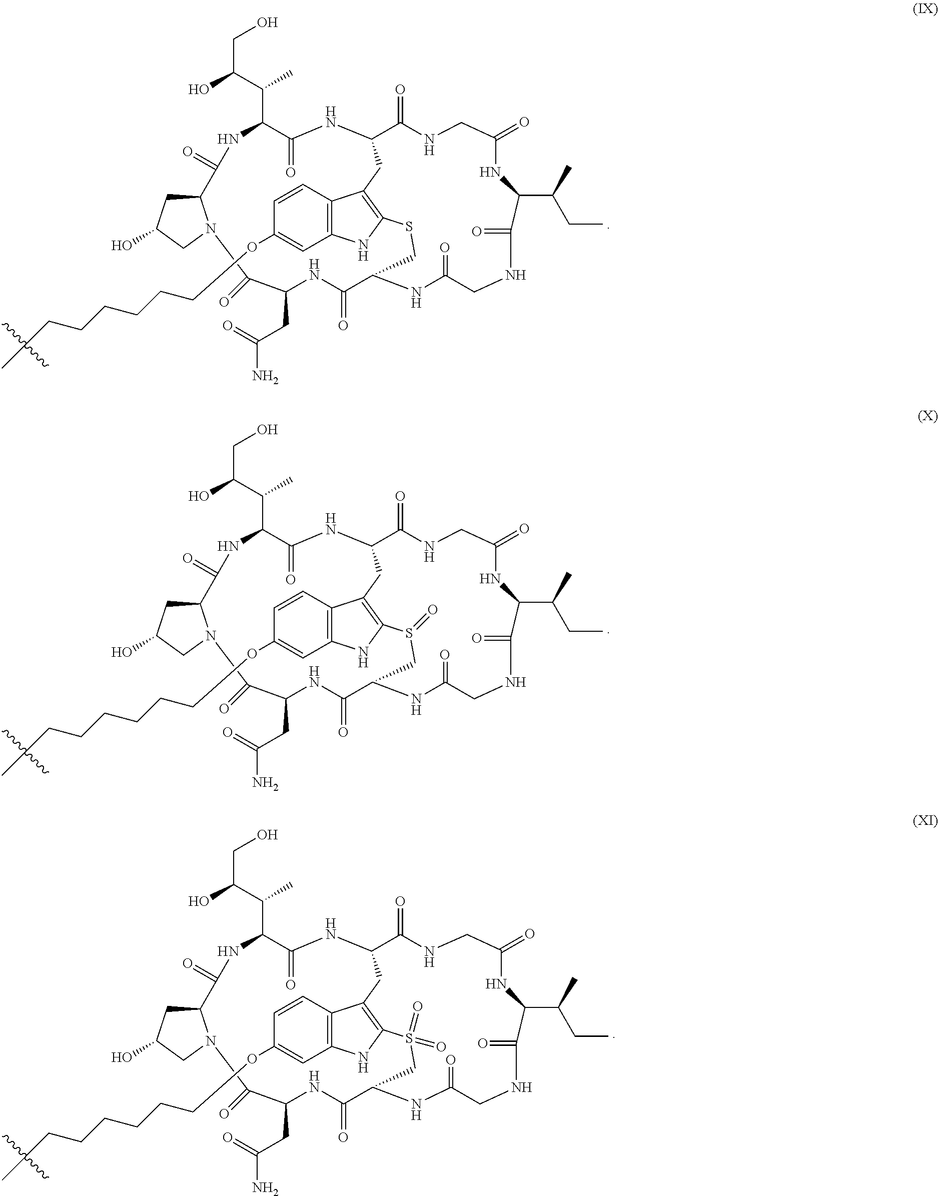

According to one embodiment, the conjugates of the invention are preferably synthesized by reacting thiol groups of antibodies with a compound according to any one of formula XIIa to XXIIa containing a maleimide moiety as reactive cap. The term “reactive cap” as used herein refers to a chemical moiety which reactacts with e.g. thiol groups of the antibody to covalenty link the the compounds of formula (XIIa) to (XXIIa) to the antibody.

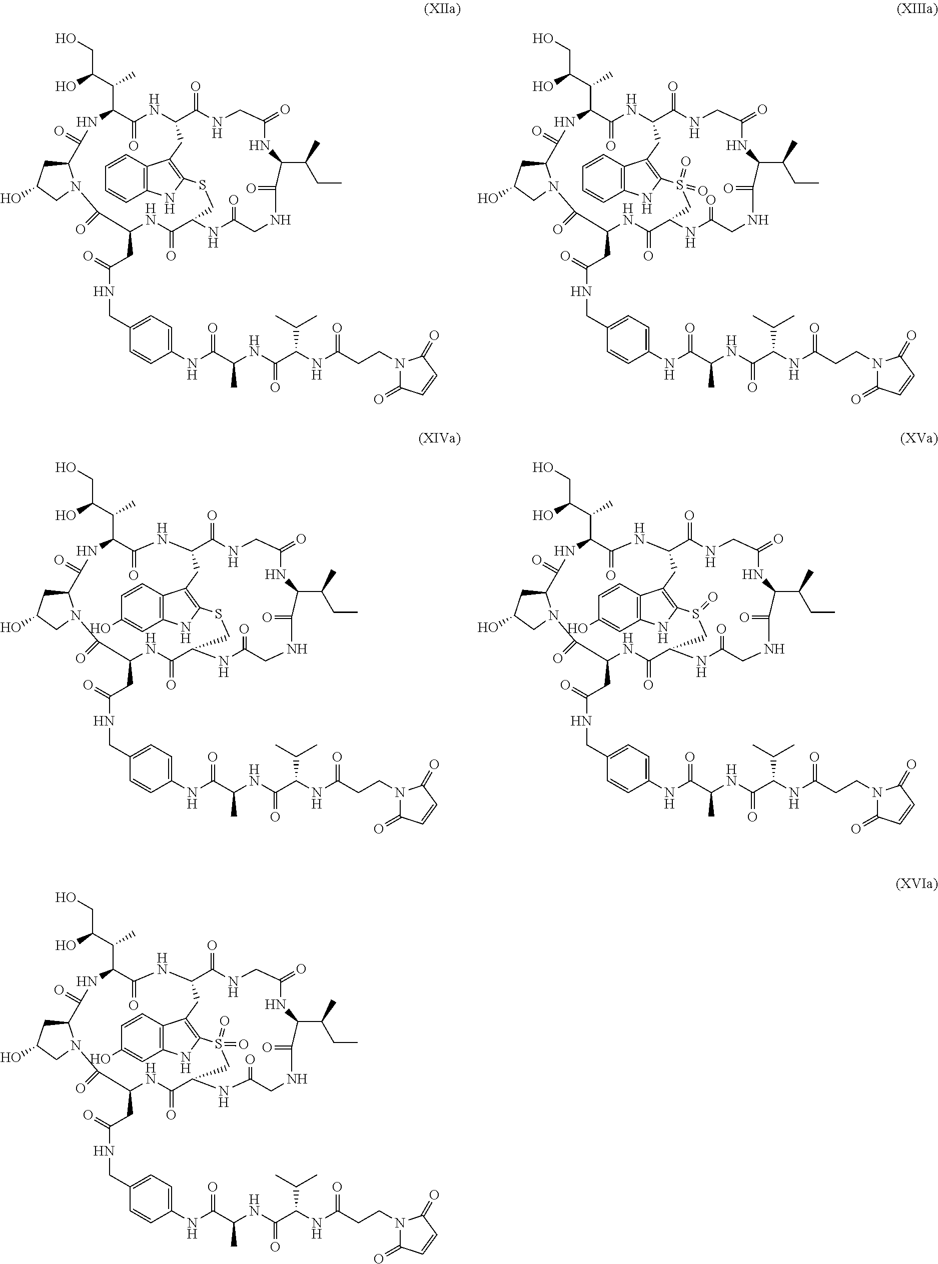

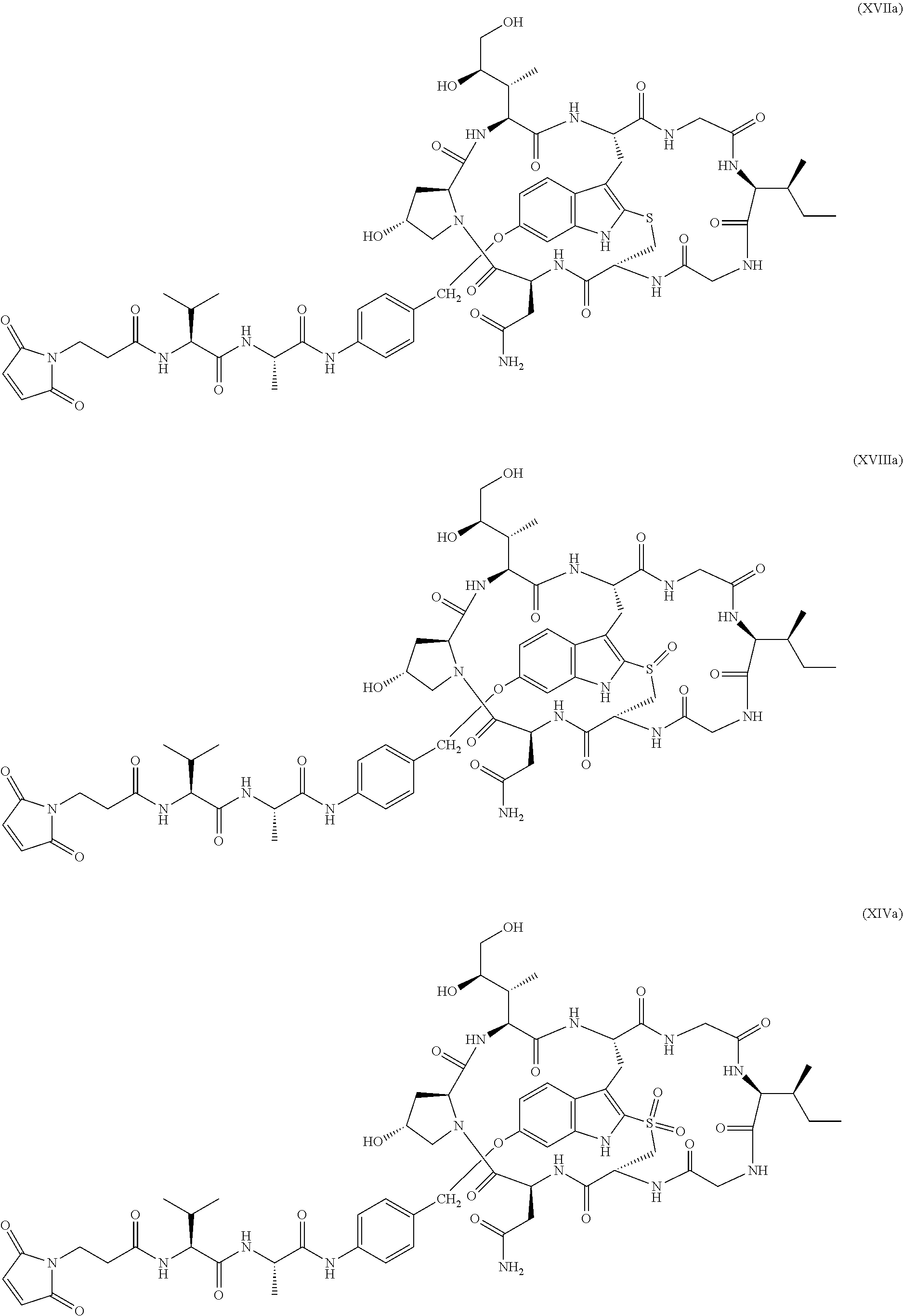

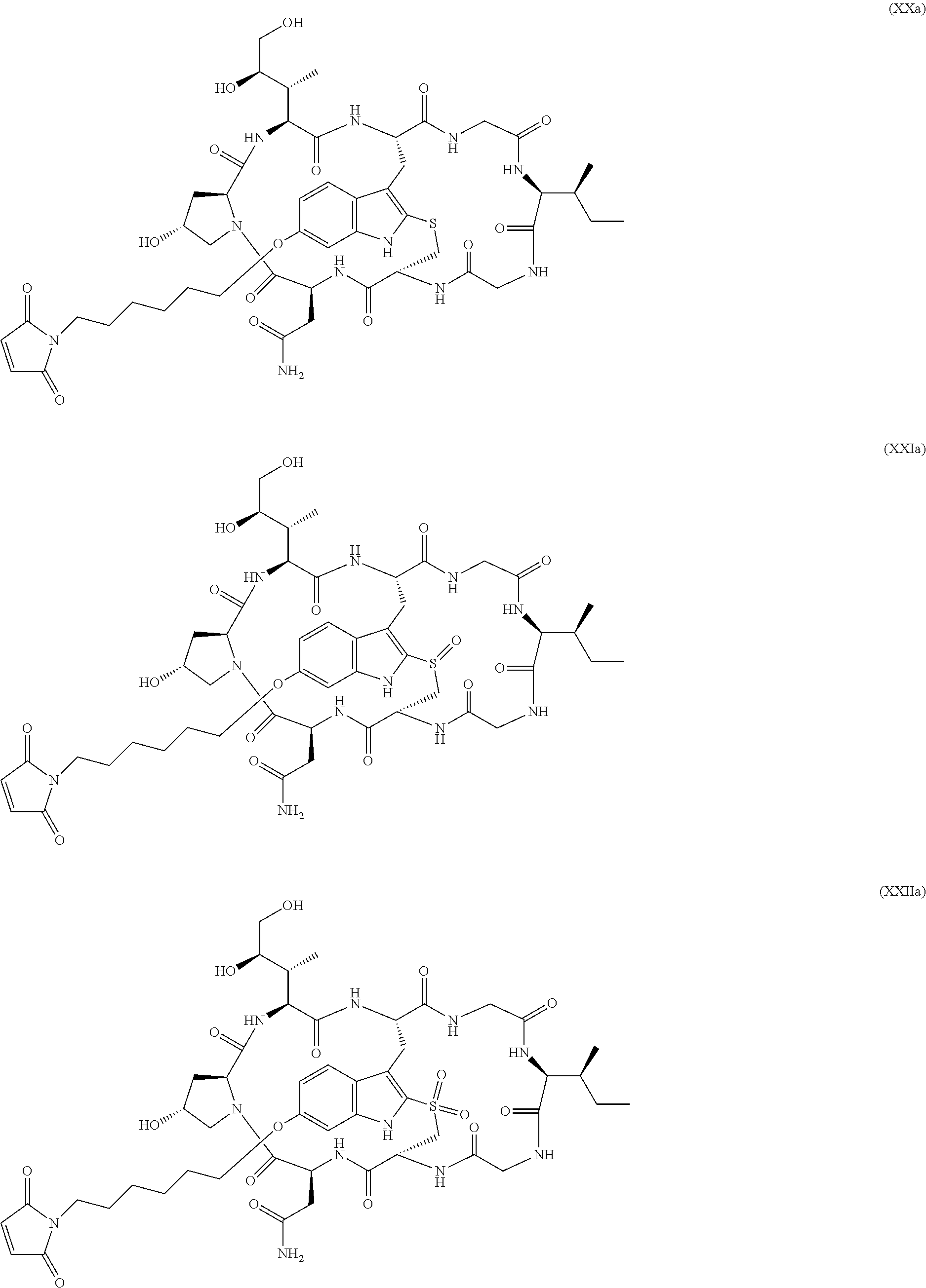

According to some embodiments, the compounds according to formula (XIIa)-(XXIIa) of the invention are used for generating or manufacturing antibody-drug conjugates (ADCs), more specifically antibody-targeted amatoxin conjugates. The ADCs of the invention as disclosed herein may also be referred to as “ATACs”. For example, compounds (XIIa) to (XXIIa) are reacted with target binding moieties, such as e.g. antibodies, e.g. human IgG1 antibodies, under suitable conditions to form ATACs. The compounds of the invention according to formula (XIIa)-(XXIIa) can e.g. be reacted with target binding moieties, such as e.g. antibodies, directed against PSMA, CD19, CD37, CD269, sialyl Lewis^{a}, HER-2/neu, epithelial cell adhesion molecule (EpCAM) to yield the corresponding ATACs. Coupling of compounds (XIIa)-(XXIIa) of the invention to a target binding moiety, or antibody may e.g. be done as disclosed in WO2018/115466 A1 to yield ATACs comprising one of compounds (XIIb)-(XXIIb) of the invention.

According to preferred embodiments of the present invention, said conjugates of the invention are compound according to any one of formula (XIIb) to (XXIIb) as disclosed hereinbelow:

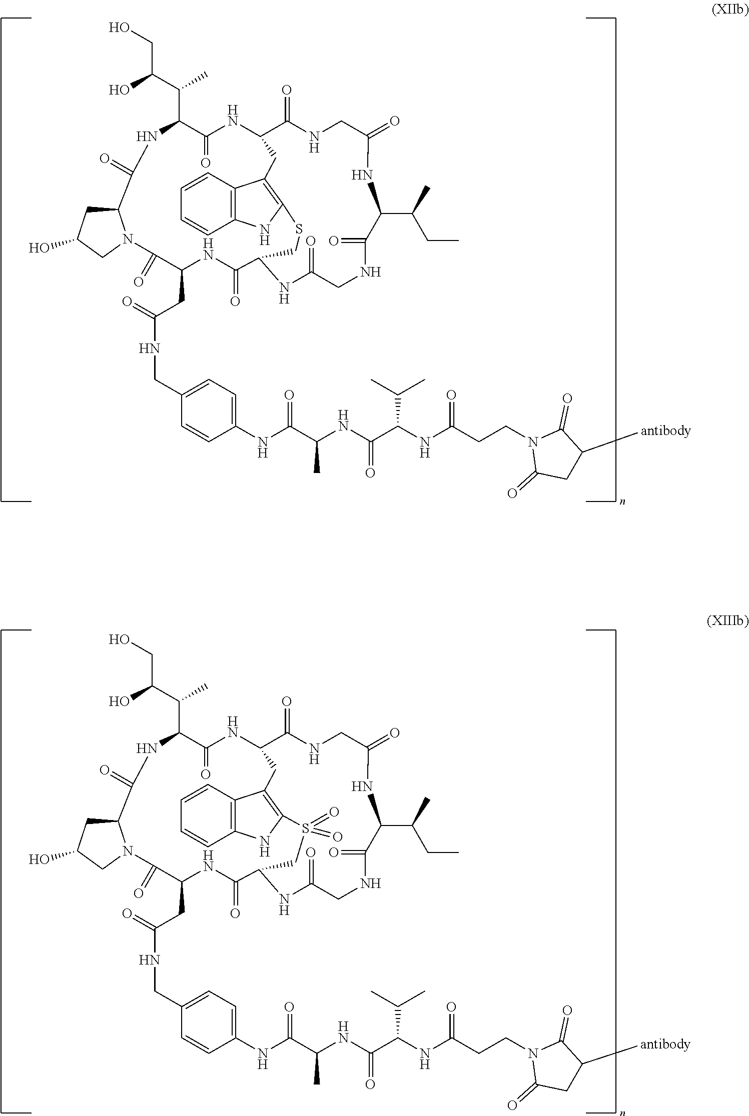

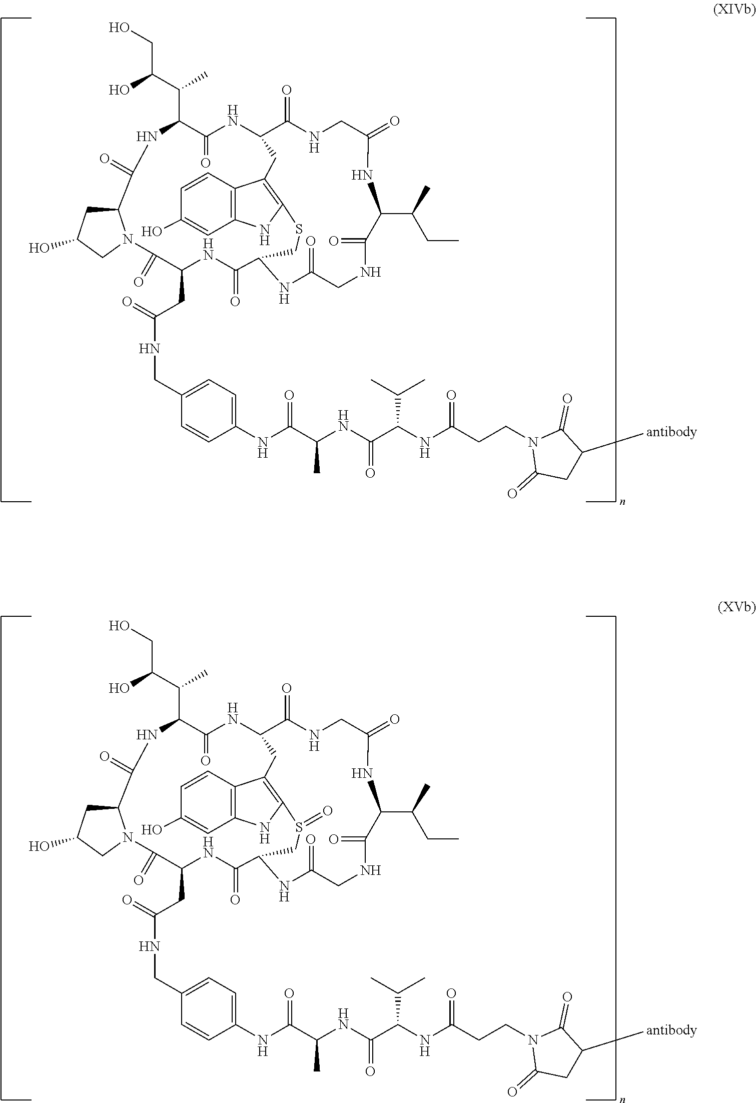

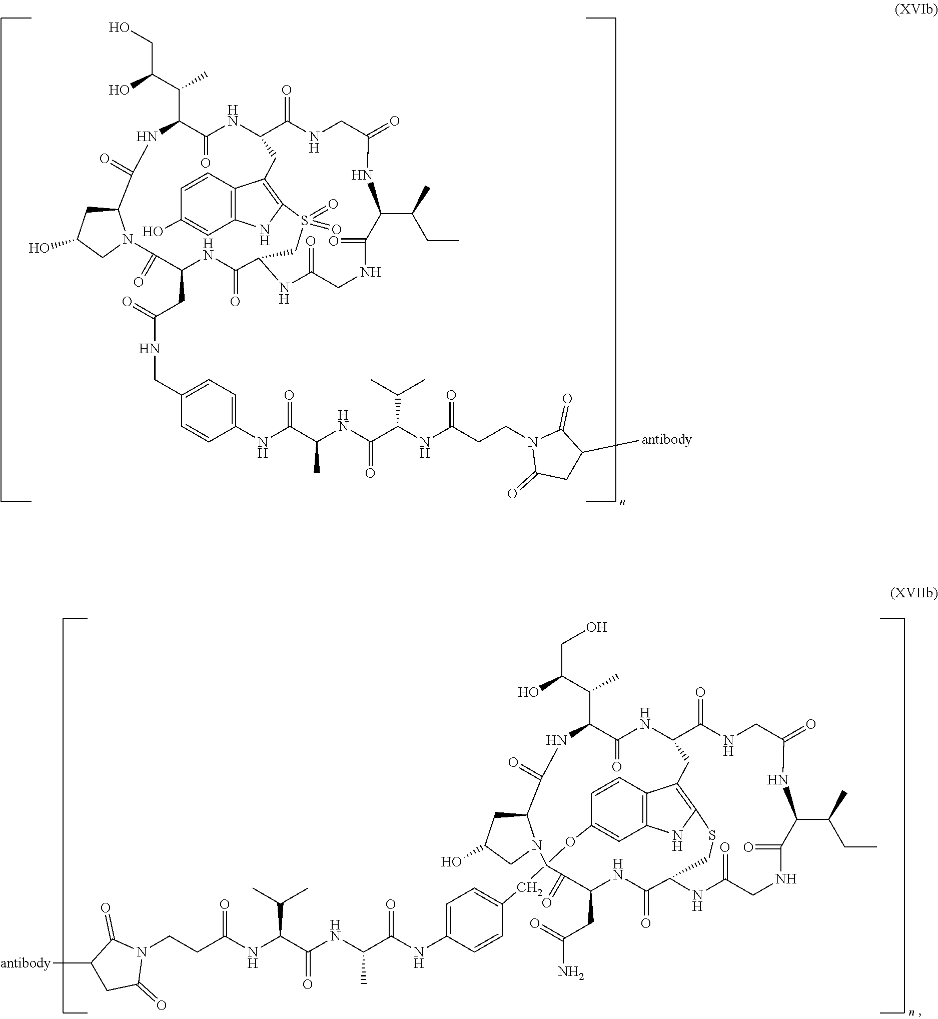

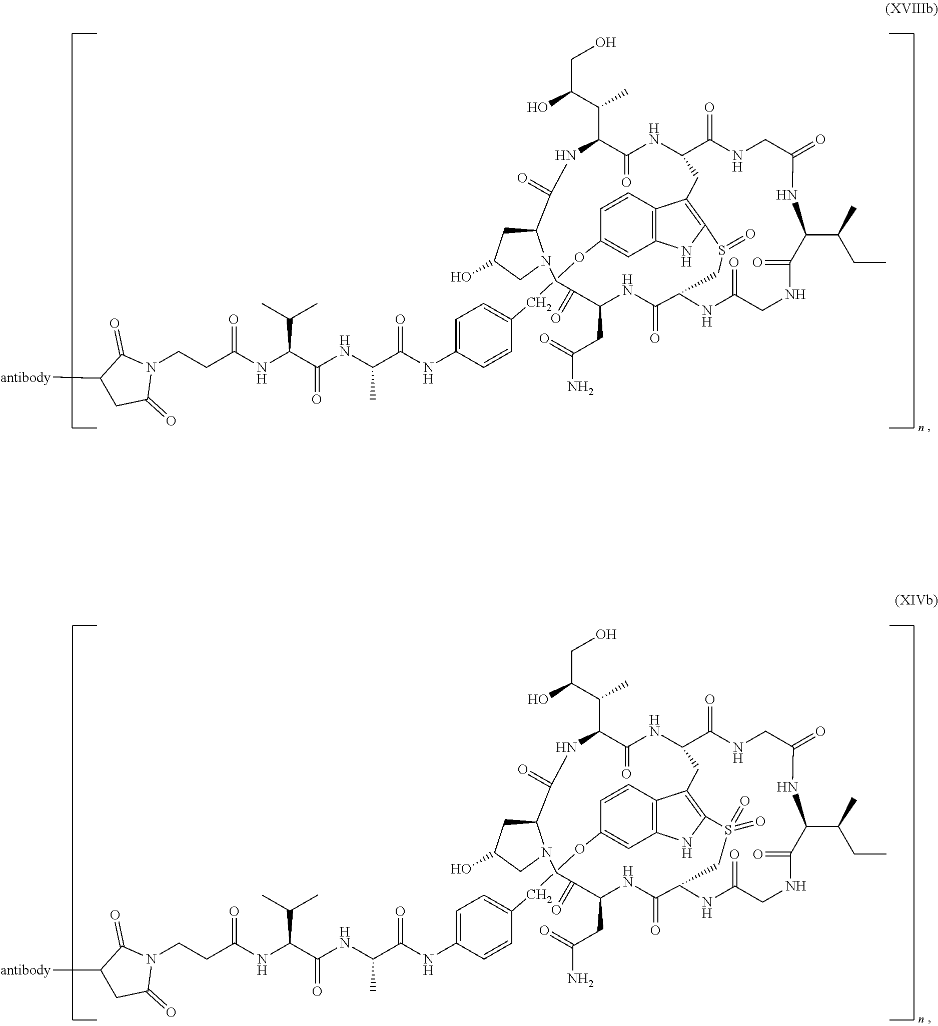

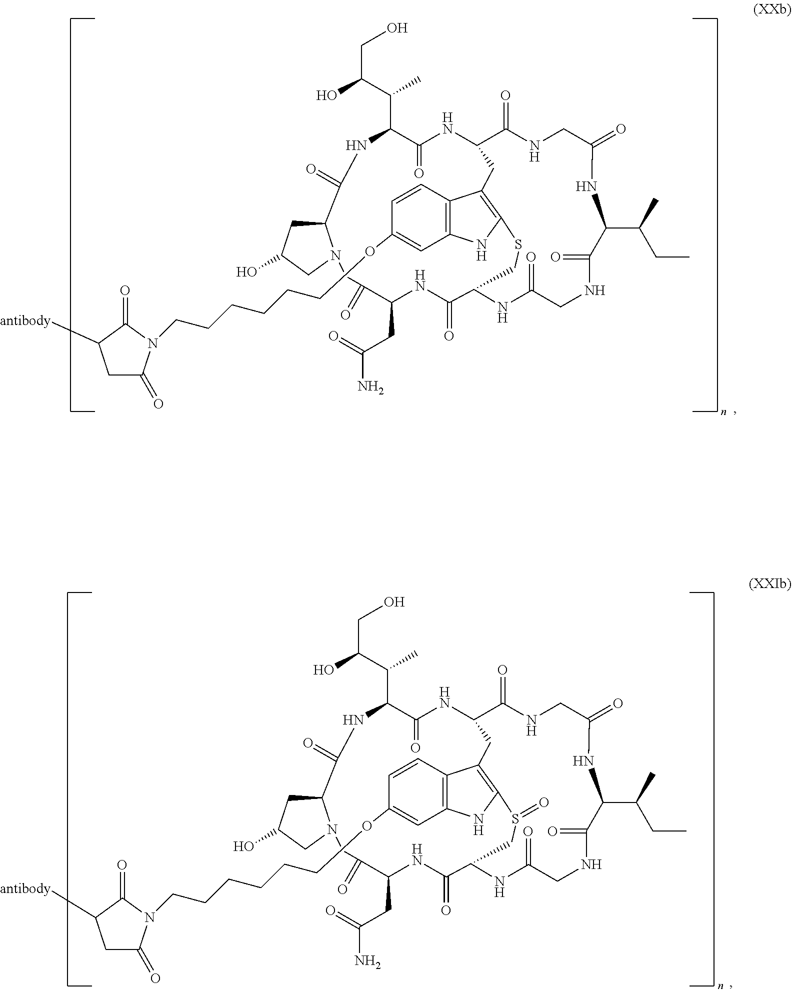

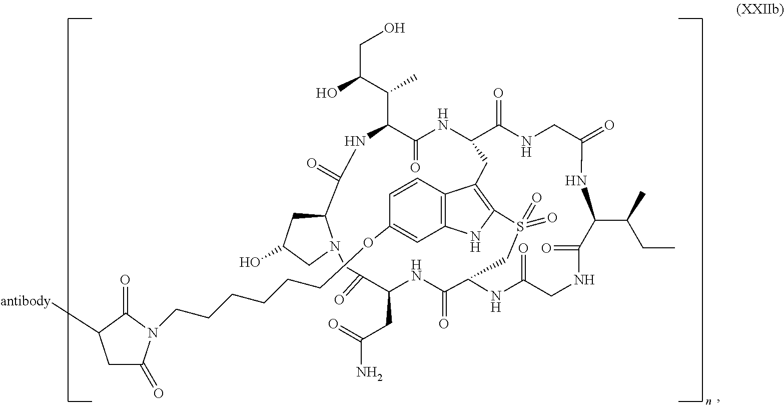

wherein n is preferably from 1 to 10, preferably from 1, 2, 3 to 4, preferably from 1, 2 to 5, preferably from 4 to 7, preferably from 8 to 10.

Furthermore, according to preferred embodiments of the present invention, said conjugate is a compound according to any one of formula XIII to XXII:

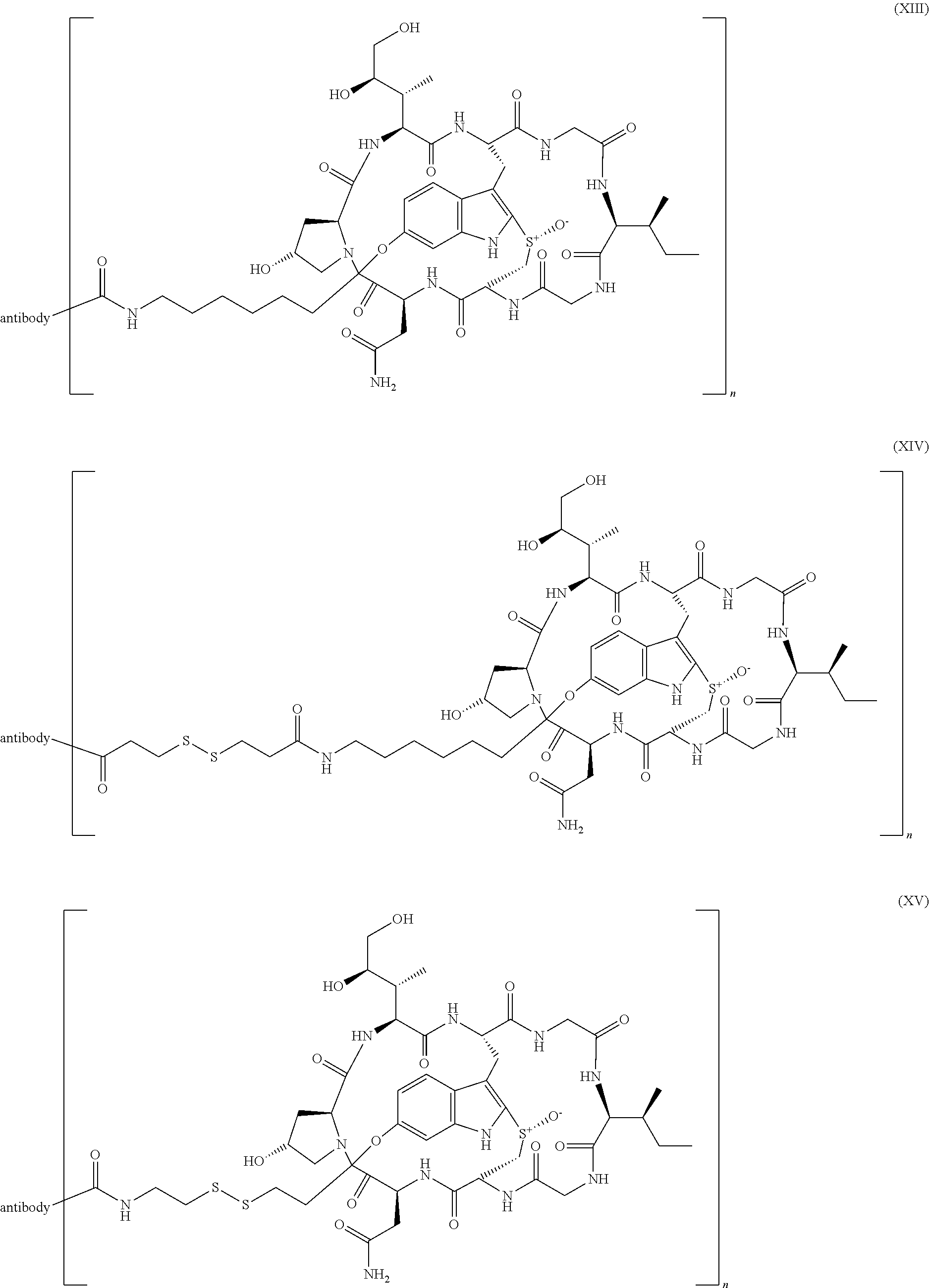

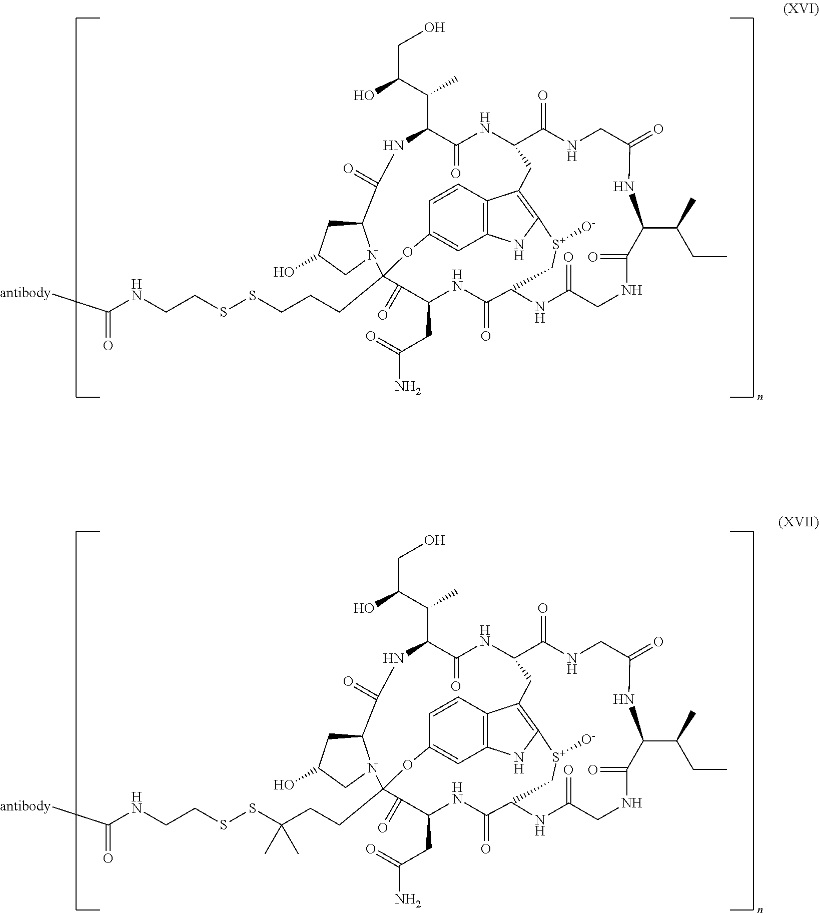

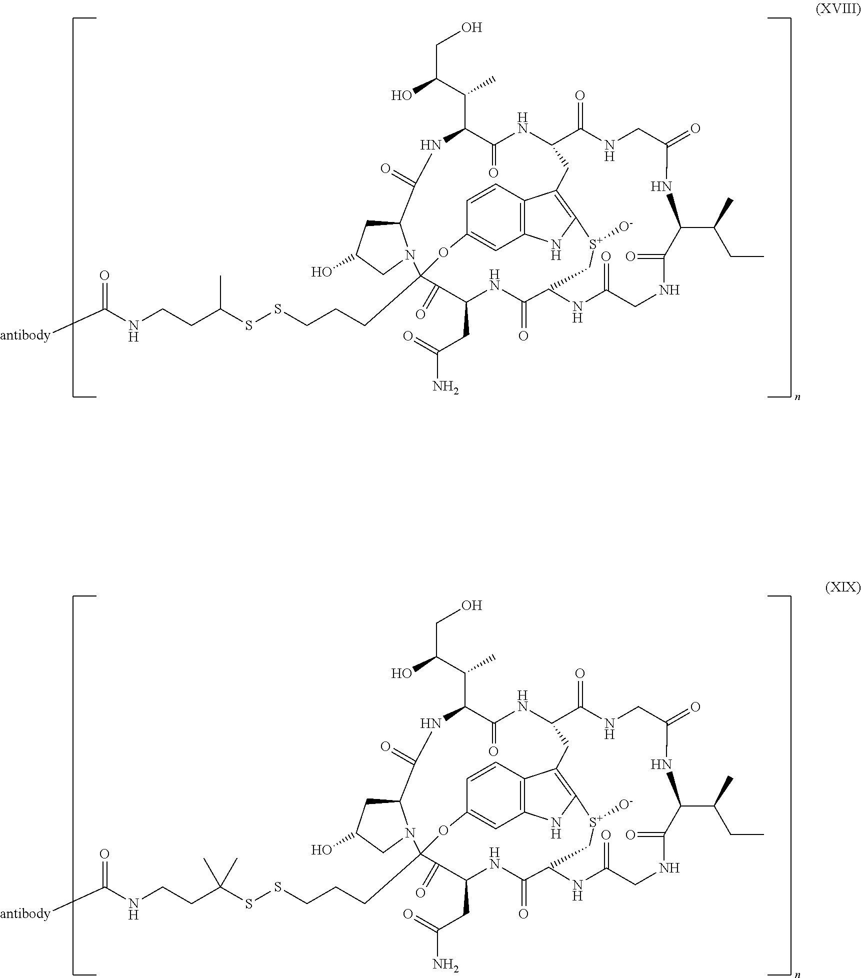

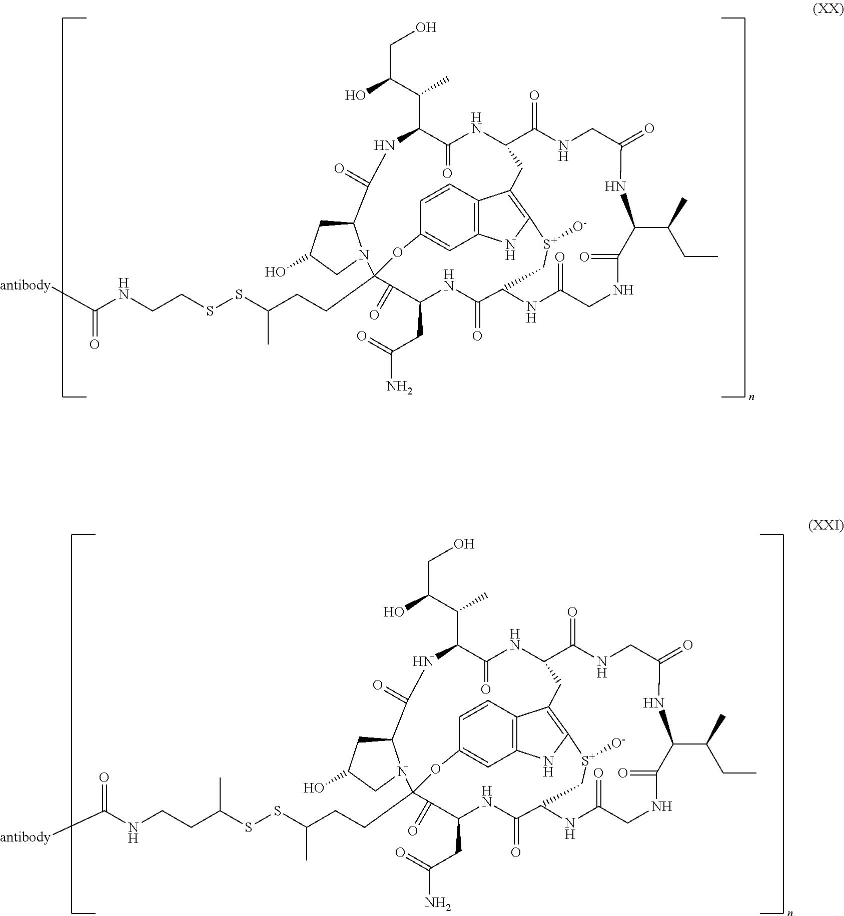

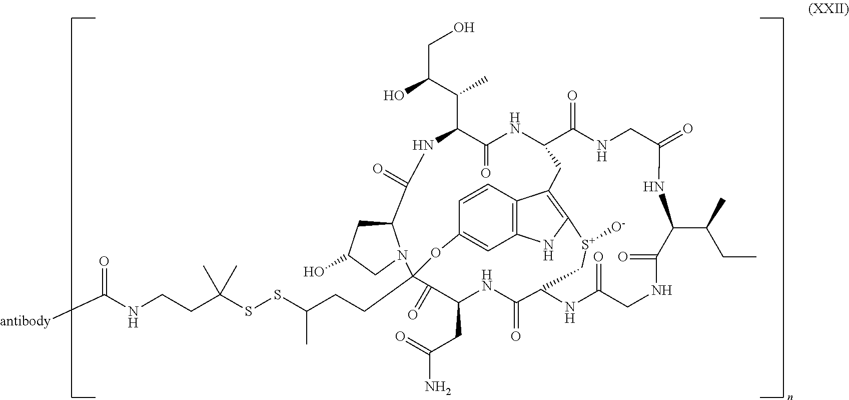

wherein the amatoxin linker moieties are coupled to ε-amino groups of naturally occurring lysine residues of said antibody, and wherein n is preferably from 1 to 8, preferably from 1, 2, 3 to 4, preferably from 2 to 5, preferably from 5 to 7.

Furthermore, according to preferred embodiments of the present invention, said conjugate is a compound according to any one of formula XXIII, XXIV, XIIIb, XXIIIb, XXb, and XVIb:

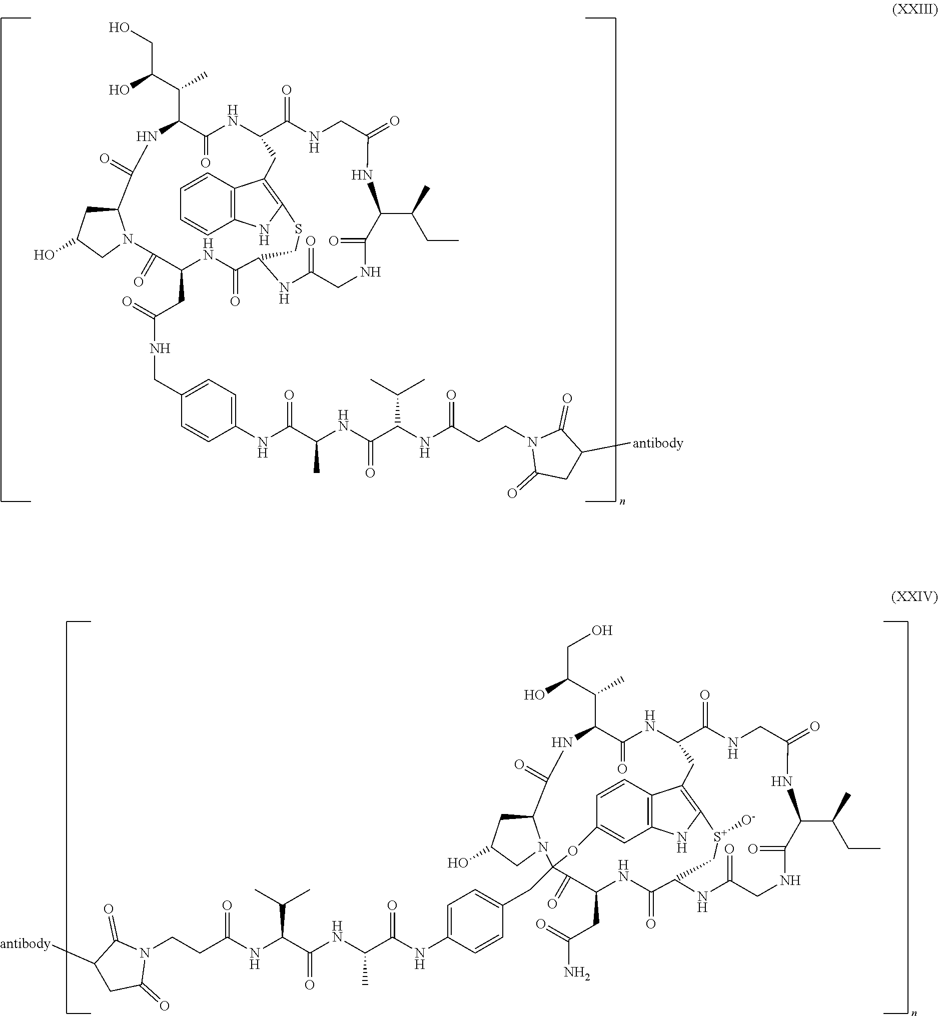

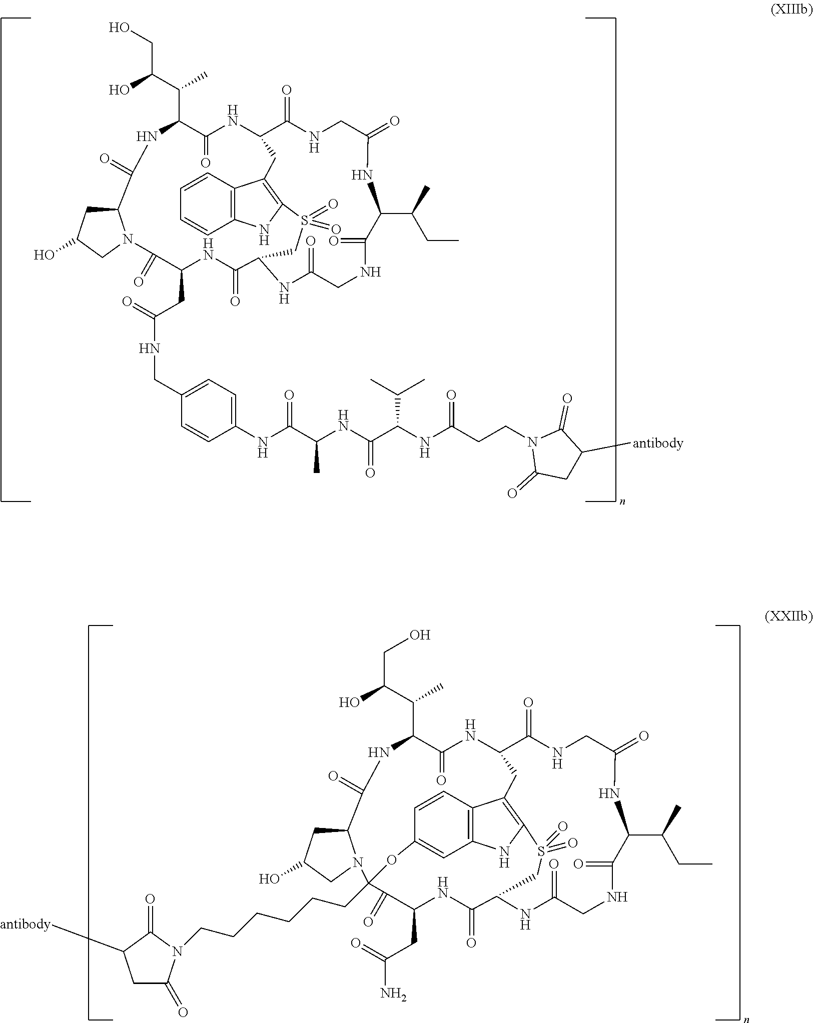

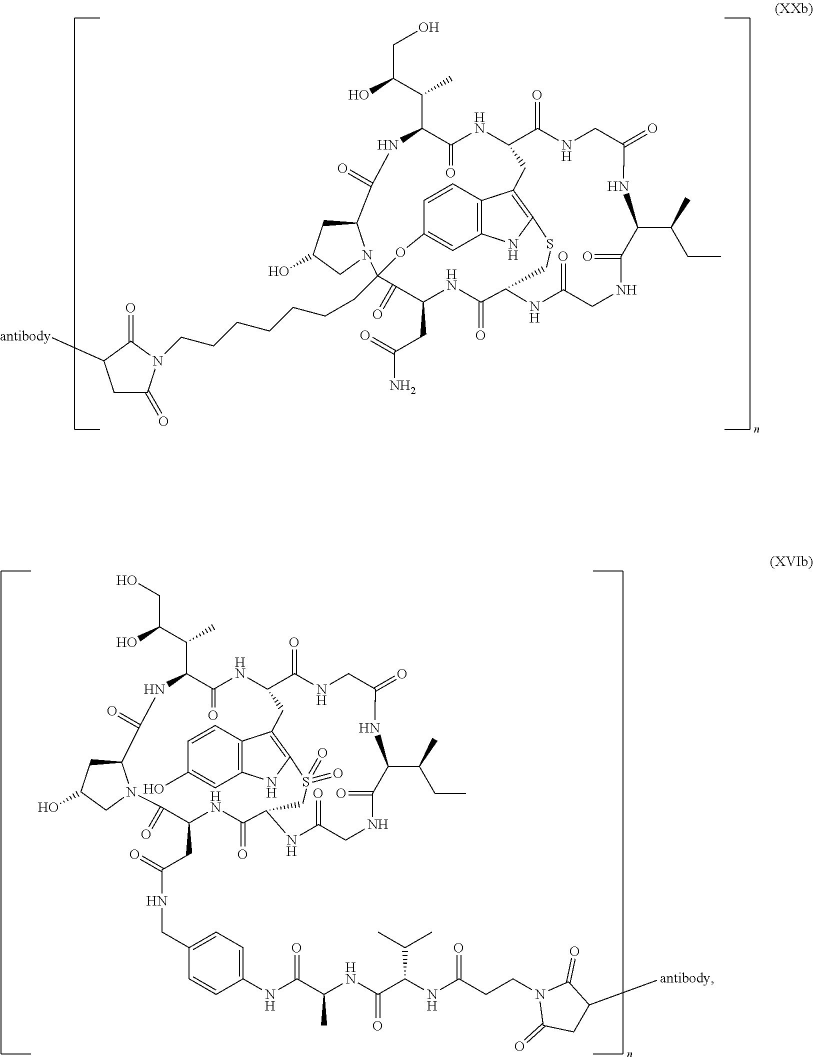

wherein the amatoxin linker moieties are coupled to the thiol groups of cysteine residues of the antibody, and wherein n is preferably from 1 to 10, or e.g. wherein n is from 2, 4 to 6, more preferably wherein n is 1, 2, 4, or 8.

According to preferred embodiments, of the present invention, said composition comprises at least one one of the immune checkpoint inhibitors avelumab, nivolumab, pembrolizumab, durvalumab, or ipilimumab and a conjugate comprising an antibody directed against HER2 (αHER2-LALA-D265C) and an amatoxin according to formula XXIb, XXIIb, XIIb, XIIIb, XVIIIb, or XIXb, wherein the antibody as denoted in the respective amatoxin structures as disclosed above is the αHER2-LALA-D265C as disclosed herein. Thus, according to preferred embodiments of the invention, said composition comprises one of

| | | | immune |
|---|---|---|---|
| | | | checkpoint |
| | Antibody | conjugate | inhibitor |
| 1 | α HER2-LALA- | XXIb, XXIIb, XIIb, XIIIb, | Avelumab |
| | D265C | XVIIIb, or XIXb | |
| 2 | α HER2-LALA- | XXIb, XXIIb, XIIb, XIIIb, | Nivolumab |
| | D265C | XVIIIb, or XIXb | |
| 3 | α HER2-LALA- | XXIb, XXIIb, XIIb, XIIIb, | Pembrolizumab |
| | D265C | XVIIIb, or XIXb | |
| 4 | α HER2-LALA- | XXIb, XXIIb, XIIb, XIIIb, | Ipilimumab |
| | D265C | XVIIIb, or XIXb | |
| 5 | α HER2-LALA- | XXIb, XXIIb, XIIb, XIIIb, | durvalumab |
| | D265C | XVIIIb, or XIXb | |
| 6 | α HER2-LALA- | XXIb, XXIIb, XIIb, XIIIb, | Avelumab + |
| | D265C | XVIIIb, or XIXb | Ipilimumab |

Accordingly, the composition of the invention comprises the antibody as disclosed above coupled to one amatoxin as disclosed above and at least one immune checkpoint inhibitor as disclosed above.

According to particularly preferred embodiments of the present invention, said composition comprises at least one one of the immune checkpoint inhibitors avelumab, nivolumab, pembrolizumab, durvalumab, or ipilimumab and a conjugate comprising an antibody directed against CD19 (chiBCE19-D265C) and an amatoxin according to formula XXIII, XXII, XXb, XXIIb, XXIV, XIIIb, or XVIb, wherein the antibody as denoted in the respective amatoxin structures as disclosed above is CD19 (chiBCE19-D265C). Thus, according to particularly preferred embodiments of the invention, said composition comprises one of:

| | | | immune |
|---|---|---|---|
| | | | checkpoint |
| | Antibody | Conjugate | inhibitor |
| 1 | α CD19 | XXIII, XXII, XXb, | Avelumab |
| | (chiBCE19-D265C) | XXIIb, XXIV, XIIIb, | |
| | | or XVIb | |
| 2 | α CD19 | XXIII, XXII, XXb, | Nivolumab |
| | (chiBCE19-D265C) | XXIIb, XXIV, | XIIIb, |
| | | or XVIb | |
| 3 | α CD19 | XXIII, XXII, XXb, | pembrolizumab |
| | (chiBCE19-D265C) | XXIIb, XXIV, XIIIb, | |
| | | or XVIb | |
| 4 | α CD19 | XXIII, XXII, XXb, | Ipilimumab |
| | (chiBCE19-D265C) | XXIIb, XXIV, XIIIb, | |
| | | or XVIb | |
| 5 | α CD19 | XXIII, XXII, XXb, | durvalumab |
| | (chiBCE19-D265C) | XXIIb, XXIV, XIIIb, | |
| | | or XVIb | |
| 6 | α CD19 | XXIII, XXII, XXb, | Avelumab + |
| | (chiBCE19-D265C) | XXIIb, XXIV, XIIIb, | ipilimumab |
| | | or XVIb | |

Accordingly, the composition of the invention comprises the antibody as disclosed above coupled to one amatoxin as disclosed above and at least one immune checkpoint inhibitor as disclosed above.

According to particularly preferred embodiments, said composition according to the invention comprises at least one of the immune checkpoint inhibitors avelumab, nivolumab, pembrolizumab, durvalumab, or ipilimumab and a conjugate comprising an antibody directed against PSMA and an amatoxin according to formula XXIII, XXII, XXb, XXIIb, XXIV, XIIIb, or XVIb, wherein the antibody as denoted in the respective amatoxin structures as disclosed above is an anti-PSMA antibody. Thus, according to particularly preferred embodiments of the invention, said composition comprises one of

| | | | immune |
|---|---|---|---|
| | | | checkpoint |
| | Antibody | Conjugate | inhibitor |
| 1 | α PSMA | XXIII, XXII, XXb, XXIIb, | Avelumab |
| | | XXIV, XIIIb, or XVIb | |
| 2 | α PSMA | XXIII, XXII, XXb, XXIIb, | Nivolumab |
| | | XXIV, XIIIb, or XVIb | |
| 3 | α PSMA | XXIII, XXII, XXb, XXIIb, | Pembrolizumab |
| | | XXIV, XIIIb, or XVIb | |
| 4 | α PSMA | XXIII, XXII, XXb, XXIIb, | Ipilimumab |
| | | XXIV, XIIIb, or XVIb | |
| 5 | α PSMA | XXIII, XXII, XXb, XXIIb, | Durvalumab |
| | | XXIV, XIIIb, or XVIb | |
| 6 | α PSMA | XXIII, XXII, XXb, XXIIb, | Avelumab + |
| | | XXIV, XIIIb, or XVIb | Ipilimumab |

Accordingly, the composition of the invention comprises the antibody as disclosed above coupled to one amatoxin as disclosed above and at least one immune checkpoint inhibitor as disclosed above.

According to particularly preferred embodiments, the anti-PSMA antibody of the conjugate of the invention as disclosed above is an anti-PSMA antibody as disclosed in WO 2020/025564. For example, the antibody of the conjugate of the invention as disclosed herein is one of 3-F11-var1, 3-F11-var2, 3-F11-var3, 3-F11-var4, 3-F11-var5, 3-F11-var6, 3-F11-var7, 3-F11-var8, 3-F11-var9, 3-F11-var10, 3-F11-var11, 3-F11-var12, 3-F11-var13, 3-F11-var14, 3-F11-var115, or 3-F11-var16, preferably, the antibody of the conjugate of the invention is one of 3-F11-var1, 3-F11-var13, or 3-F11-var16 as disclosed in WO 2020/025564.

According to particularly preferred embodiments, the anti-PSMA antibody as disclosed above for use in the composition of the invention comprises at least one mutation in its Fc region selected from L234A, L235A and D265C (numbering according to EU nomenclature), preferably L234A, L235A and D265C.

According to particularly preferred embodiments, said composition according to the invention comprises at least one one of the immune checkpoint inhibitors avelumab, nivolumab, pembrolizumab, durvalumab, or ipilimumab and a conjugate comprising an antibody directed against CD37 and an amatoxin according to formula XXIII, XXII, XXb, XXIIb, XXIV, XIIIb, or XVIb, wherein the antibody as denoted in the respective amatoxin structures as disclosed above is an anti-CD37 antibody. Thus, according to particularly preferred embodiments of the invention, said composition comprises one of

| | | | immune |
|---|---|---|---|
| | | | checkpoint |
| | Antibody | Conjugate | inhibitor |
| 1 | α CD37 | XXIII, XXII, XXb, XXIIb, | Avelumab |
| | | XXIV, XIIIb, or XVIb | |
| 2 | α CD37 | XXIII, XXII, XXb, XXIIb, | Nivolumab |
| | | XXIV, XIIIb, or XVIb | |
| 3 | α CD37 | XXIII, XXII, XXb, XXIIb, | Pembrolizumab |
| | | XXIV, XIIIb, or XVIb | |
| 4 | α CD37 | XXIII, XXII, XXb, XXIIb, | Ipilimumab |
| | | XXIV, XIIIb, or XVIb | |
| 5 | α CD37 | XXIII, XXII, XXb, XXIIb, | Durvalumab |
| | | XXIV, XIIIb, or XVIb | |
| 6 | α CD37 | XXIII, XXII, XXb, XXIIb, | Avelumab + |
| | | XXIV, XIIIb, or XVIb | Ipilimumab |

Accordingly, the composition of the invention comprises the antibody as disclosed above coupled to one amatoxin as disclosed above and at least one immune checkpoint inhibitor as disclosed above.

According to particularly preferred embodiments, the anti-CD37 antibody as disclosed above for use in the composition of the invention comprises at least one mutation in its Fc region selected from L234A, L235A and D265C (numbering according to EU nomenclature), preferably L234A, L235A and D265C.

According to particularly preferred embodiments, said composition according to the invention comprises at least one of the immune checkpoint inhibitors avelumab, nivolumab, pembrolizumab, durvalumab, or ipilimumab and a conjugate comprising an antibody directed against CD269 and an amatoxin according to formula XXIII, XXII, XXb, XXIIb, XXIV, XIIIb, or XVIb, wherein the antibody as denoted in the respective amatoxin structures as disclosed above is an anti-CD269 antibody. Thus, according to particularly preferred embodiments of the invention, said composition comprises one of

| | | | immune |
|---|---|---|---|
| | | | checkpoint |
| | Antibody | Conjugate | inhibitor |
| 1 | α CD269 | XXIII, XXII, XXb, XXIIb, | Avelumab |
| | | XXIV, XIIIb, or XVIb | |
| 2 | α CD269 | XXIII, XXII, XXb, XXIIb, | Nivolumab |
| | | XXIV, XIIIb, or XVIb | |
| 3 | α CD269 | XXIII, XXII, XXb, XXIIb, | pembrolizumab |
| | | XXIV, XIIIb, or XVIb | |
| 4 | α CD269 | XXIII, XXII, XXb, XXIIb, | Ipilimumab |
| | | XXIV, XIIIb, or XVIb | |
| 5 | α CD269 | XXIII, XXII, XXb, XXIIb, | Durvalumab |
| | | XXIV, XIIIb, or XVIb | |
| 5 | α CD269 | XXIII, XXII, XXb, XXIIb, | Avelumab + |
| | | XXIV, XIIIb, or XVIb | ipilimumab |

Accordingly, the composition of the invention comprises the antibody as disclosed above coupled to one amatoxin as disclosed above and at least one immune checkpoint inhibitor as disclosed above.

According to particularly preferred embodiments, the anti-CD269 antibody of the conjugate of the invention as disclosed above is the humanized antibody J22.9-ISY as disclosed in WO2018/115466 and wherein the conjugate comprises an amatoxin linker-moiety according to formula (I), or wherein the conjugate is represented by formula (XIIb) as disclosed herein.

According to some embodiments, the anti-CD269 antibody as disclosed above for use in the composition of the invention may e.g. comprise at least one mutation in its Fc region selected from L234A, L235A and D265C (numbering according to EU nomenclature), preferably the Fc region of said antibody comprises the mutations L234A, L235A and D265C.

According to particularly preferred embodiments, said composition according to the invention comprises at least one of the immune checkpoint inhibitors avelumab, nivolumab, pembrolizumab, durvalumab, or ipilimumab and a conjugate comprising an antibody directed against sialyl Lewis^{a} and an amatoxin according to formula XXIII, XXII, XXb, XXIIb, XXIV, XIIIb, or XVIb, wherein the antibody as denoted in the respective amatoxin structures as disclosed above is an anti-α sialyl Lewis^{a} antibody Thus, according to particularly preferred embodiments of the invention, said composition comprises one of

| | | | immune |
|---|---|---|---|
| | | | checkpoint |
| | Antibody | conjugate | inhibitor |
| 1 | α sialyl Lewis^{a} | XXIII, XXII, XXb, XXIIb, | Avelumab |
| | | XXIV, XIIIb, or XVIb | |
| 2 | α sialyl Lewis^{a} | XXIII, XXII, XXb, XXIIb, | Nivolumab |
| | | XXIV, XIIIb, or XVIb | |
| 3 | α sialyl Lewis^{a} | XXIII, XXII, XXb, XXIIb, | pembrolizumab |
| | | XXIV, XIIIb, or XVIb | |
| 4 | α sialyl Lewis^{a} | XXIII, XXII, XXb, XXIIb, | Ipilimumab |
| | | XXIV, XIIIb, or XVIb | |
| 5 | α sialyl Lewis^{a} | XXIII, XXII, XXb, XXIIb, | Durvalumab |
| | | XXIV, XIIIb, or XVIb | |
| 6 | α sialyl Lewis^{a} | XXIII, XXII, XXb, XXIIb, | Avelumab + |
| | | XXIV, XIIIb, or XVIb | Ipilimumab |

Accordingly, the composition of the invention comprises the antibody as disclosed above coupled to one amatoxin as disclosed above and at least one immune checkpoint inhibitor as disclosed above.

According to some embodiments, the anti-sialyl Lewis^{a} antibody as disclosed above for use in the composition of the invention comprises at least one mutation in its Fc region selected from L234A, L235A and D265C (numbering according to EU nomeclature), preferably L234A, L235A and D265C.

According to particularly preferred embodiments, said composition according to the invention comprises at least one one of the immune checkpoint inhibitors avelumab, nivolumab, pembrolizumab, durvalumab or ipilimumab and a conjugate comprising an antibody directed against EpCAM and an amatoxin according to formula XXIII, XXII, XXb, XXIIb, XXIV, XIIIb, or XVIb, wherein the antibody as denoted in the respective amatoxin structures as disclosed above is an anti-EpCAM antibody. Thus, according to particularly preferred embodiments of the invention, said composition comprises one of

| | | | immune |
|---|---|---|---|
| | | | checkpoint |
| | Antibody | Conjugate | inhibitor |
| 1 | α EpCAM | XXIII, XXII, XXb, XXIIb, | Avelumab |
| | | XXIV, XIIIb, or XVIb | |
| 2 | α EpCAM | XXIII, XXII, XXb, XXIIb, | Nivolumab |
| | | XXIV, XIIIb, or XVIb | |
| 3 | α EpCAM | XXIII, XXII, XXb, XXIIb, | pembrolizumab |
| | | XXIV, XIIIb, or XVIb | |
| 4 | α EpCAM | XXIII, XXII, XXb, XXIIb, | Ipilimumab |
| | | XXIV, XIIIb, or XVIb | |
| 5 | α EpCAM | XXIII, XXII, XXb, XXIIb, | Durvalumab |
| | | XXIV, XIIIb, or XVIb | |
| 6 | α EpCAM | XXIII, XXII, XXb, XXIIb, | Avelumab + |
| | | XXIV, XIIIb, or XVIb | ipilimumab |

Accordingly, the composition of the invention comprises the antibody as disclosed above coupled to one amatoxin as disclosed above and at least one immune checkpoint inhibitor as disclosed above.

According to some embodiments, the anti-EpCAM antibody as disclosed above for use in the composition of the invention comprises at least one mutation in its Fc region selected from L234A, L235A and D265C (numbering according to EU nomenclature), preferably L234A, L235A and D265C.

According to particularly preferred embodiments the conjugate for use in the composition of the invention as disclosed above comprises or is according to formula XIIb, XIVb, XVIIIb, XXb, or XVIIb. Thus, according to particularly preferred embodiments, said composition according to the invention comprises at least one one of the immune checkpoint inhibitors avelumab, nivolumab, pembrolizumab, durvalumab, or ipilimumab and a conjugate according to formula XXIII, XXII, XXb, XXIIb, XXIV, XIIIb, or XVIb. Thus, according to particularly preferred embodiments of the invention, said composition comprises at least one of

| | | immune checkpoint |
|---|---|---|
| | Conjugate | inhibitor |
| 1 | XXIII, XXII, XXb, XXIIb, | |
| | XXIV, XIIIb, or XVIb | Avelumab |
| 2 | XXIII, XXII, XXb, XXIIb, | |
| | XXIV, XIIIb, or XVIb | Nivolumab |
| 3 | XXIII, XXII, XXb, XXIIb, | |
| | XXIV, XIIIb, or XVIb | pembrolizumab |
| 4 | XXIII, XXII, XXb, XXIIb, | |
| | XXIV, XIIIb, or XVIb | Ipilimumab |
| 5 | XXIII, XXII, XXb, XXIIb, | |
| | XXIV, XIIIb, or XVIb | Durvalumab |
| 6 | XXIII, XXII, XXb, XXIIb, | |
| | XXIV, XIIIb, or XVIb | Avelumab + |
| | | ipilimumab |

Accordingly, the composition of the invention comprises at least a conjugate as disclosed above and at least one immune checkpoint inhibitor as disclosed above.

According to one embodiment, the present invention pertains a conjugate according to formula (XIIb), (XIIIb), (XIVb), (XVb), (XVIb), (XVIIb), (XVIIIb), (XIXb), (XXb), (XXIb), (XXIIb) for use in the manufacture of a composition of the invention.

According to one embodiment, the present invention pertains to an immune checkpoint inhibitor selected from the group of avelumab, nivolumab, pembrolizumab, Ipilimumab, or durvalumab for use in the manufacture of a composition according to the invention as disclosed herein.

According to a fourth aspect of the present invention, a pharmaceutical formulation is provided, comprising the composition (for use) as described, further comprising one or more pharmaceutically acceptable buffers, surfactants, diluents, carriers, excipients, fillers, binders, lubricants, glidants, disintegrants, adsorbents, and/or preservatives.

In aqueous form, said pharmaceutical formulation may be ready for administration, while in lyophilised form said formulation can be transferred into liquid form prior to administration, e.g., by addition of water for injection which may or may not comprise a preservative such as for example, but not limited to, benzyl alcohol, antioxidants like vitamin A, vitamin E, vitamin C, retinyl palmitate, and selenium, the amino acids cysteine and methionine, citric acid and sodium citrate, synthetic preservatives like the parabens methyl paraben and propyl paraben.

Said pharmaceutical formulation may further comprise one or more stabilizer, which may be, e.g., an amino acid, a sugar polyol, a disaccharide and/or a polysaccharide. Said pharmaceutical formulation may further comprise one or more surfactant, one or more isotonizing agents, and/or one or more metal ion chelator, and/or one or more preservative.

The pharmaceutical formulation as described herein can be suitable for at least intravenous, intramuscular or subcutaneous administration. Alternatively, said conjugate according to the present invention may be provided in a depot formulation which allows the sustained release of the biologically active agent over a certain period of time.

In still another aspect of the present invention, a primary packaging, such as a prefilled syringe or pen, a vial, or an infusion bag is provided, which comprises said formulation according to the previous aspect of the invention.

The prefilled syringe or pen may contain the formulation either in lyophilised form (which has then to be solubilised, e.g., with water for injection, prior to administration), or in aqueous form. Said syringe or pen is often a disposable article for single use only, and may have a volume between 0.1 and 20 ml. However, the syringe or pen may also be a multi-use or multi-dose syringe or pen.

Said vial may also contain the formulation in lyophilised form or in aqueous form and may serve as a single or multiple use device. As a multiple use device, said vial can have a bigger volume. Said infusion bag usually contains the formulation in aqueous form and may have a volume between 20 and 5000 ml.

In some embodiments of the present invention, the composition for use in the treatment of cancer, or the pharmaceutical formulation as described, relates to a cancer which is selected from the group consisting of melanoma, squamous and non-squamous non-small cell lung cancer, metastatic small cell lung cancer, renal cell carcinoma, Hodgkin lymphoma, B lymphocyte-associated malignancies, urothelial carcinoma, head and neck squamous cell carcinoma, Merkel cell carcinoma, hepatocellular carcinoma, gastric and gastroesophageal carcinoma, metastatic colorectal cancer, multiple myeloma, primary mediastinal B cell lymphoma, recurrent or metastatic cervical cancer, and metastatic cutaneous squamous cell carcinoma, prostate cancer, breast cancer, including triple-negative breast cancer (TNBC).

In preferred embodiments, the present invention relates to said composition or pharmaceutical formulation as disclosed herein for use in the treatment of B lymphocyte-associated malignancies, in particular for use in the treatment of non-Hodgkin's lymphoma, follicular lymphoma, diffuse large B cell non-Hodgkin's lymphoma, and chronic lymphocytic leukaemia.

In some embodiments, the present invention pertains to an immune checkpoint inhibitor as disclosed herein for use in a composition of the invention, or in a pharmaceutical composition of the invention. Accordingly, the present invention pertains to avelumab, nivolumab, pembrolizumab, ipilimumab, PD1-1, PD1-2, PD1-3, PD1-4, PD1-5, pidilizumab, cemiplimab, JTX-4014, spartalizumab, sintilimab (IBI308), dostarlimab, Toripalimab, durvalumab, or atezolizumab for use in a composition or pharmaceutical formulation according to the invention as disclosed herein.

In some embodiments, the present invention pertains to the use of avelumab, nivolumab, pembrolizumab, ipilimumab, or durvalumab in a composition or pharmaceutical formulation according to the invention as disclosed herein.

The invention further relates to a method for the treatment of cancer in a human subject in need thereof, wherein the method comprises administering to the subject a composition comprising (a) at least one immune checkpoint inhibitor and (b) at least one conjugate, wherein said conjugate is comprising (i) a target binding moiety, (ii) at least one amatoxin, and (iii) optionally at least one linker connecting said target binding moiety with said at least one amatoxin.

According to one embodiment, the method of treatment of cancer in a human subject in need thereof as disclosed above comprises administering the composition or the pharmaceutical formulation as disclosed above for the treatment of melanoma, squamous and non-squamous non-small cell lung cancer, metastatic small cell lung cancer, renal cell carcinoma, Hodgkin lymphoma, urothelial carcinoma, head and neck squamous cell carcinoma, Merkel cell carcinoma, hepatocellular carcinoma, gastric and gastroesophageal carcinoma, metastatic colorectal cancer, primary mediastinal B cell lymphoma, recurrent or metastatic cervical cancer, and metastatic cutaneous squamous cell carcinoma.

In some embodiments, the method of treating cancer as disclosed above comprises adiministering the immune checkpoint inhibitor and the conjugate of the composition as disclosed herein sequentially or simultaneously. In case of a sequential administration of the immune checkpoint inhibitor and of the conjugate of the invention the checkpoint inhibitor may be administered first, followed by the administration of the conjugate, alternatively, the conjugate of the invention may e.g. be administered first followed by the administration of the immune checkpoint inhibitor. In preferred embodiments, the immune checkpoint inhibitor and the conjugate of the invention are administered intravenously (i.v.) sequentially or simultaneously as disclosed above. The term “simultaneous administration” as used herein refers to the administration of the immune checkpoint inhibitor and of the conjugate of the invention on the same day, e.g. within 1 hour, 2 hours, 4 hours, 6 hours, 8 hours, 12 hours, 18 hours, 20 hours, or 23 hours apart from each other.

The invention further relates to the use of a composition comprising (a) at least one immune checkpoint inhibitor and (b) at least one conjugate, wherein said conjugate is comprising (i) a target binding moiety, (ii) at least one amatoxin, and (iii) optionally at least one linker connecting said target binding moiety with said at least one amatoxin, for the treatment of cancer.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Example 1: Synthesis of Linker Toxins

### Synthesis of Linker Toxin XIIIa

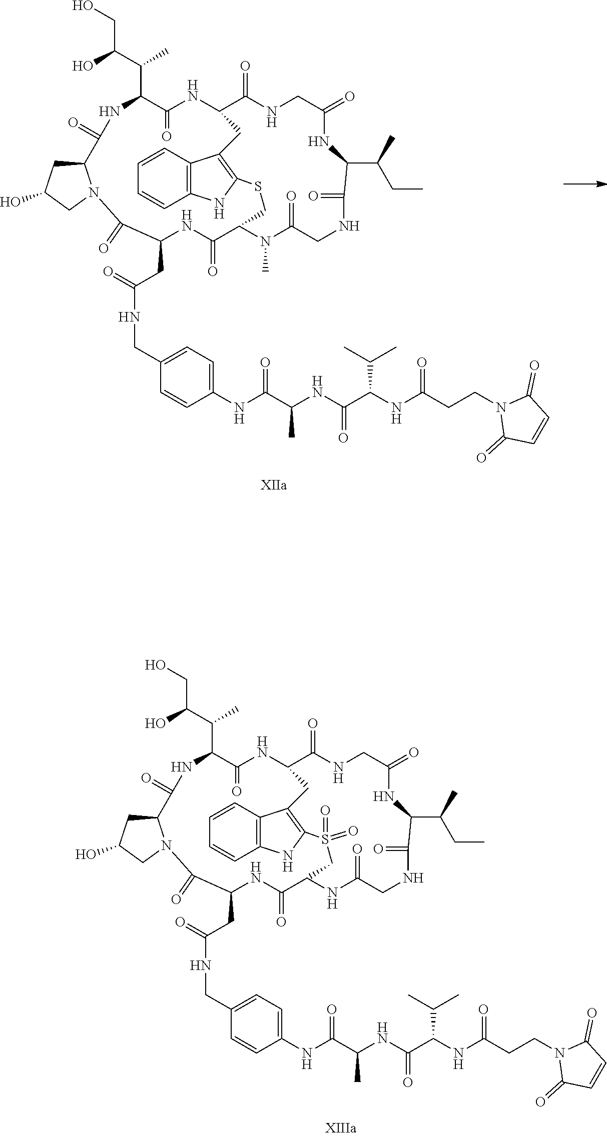

5.15 mg (3.92 μmol) Thioether XIIa (as described in WO2018/115466) were dissolved in 1000 μl acetic acid. At ambient temperature 2078.99 μl of a solution of m-CPBA (meta-chloroperbenzoic acid, 69.5%, Aldrich) in acetic acid were added (stock solution: 5.15 mg m-CPBA in 5000 μl acetic acid). After stirring for 3 h 15 min at ambient temperature the solution was dropped into 15 ml ice-cold MTBE. The whole mixture was stored for additional 1 h at −18° C. and the precipitate was isolated by centrifugation. The solid residue was washed with 15 ml ice-cold MTBE and dried. The crude product was purified by HPLC to yield 3.12 mg (59%) of the pure product. The product was freeze-dried from acetonitrile/water (1/1) to a white amorphous solid.

HPLC purification:

Prep. HPLC was carried out with a Phenomenex Luna-C18 (2), 10 μm column (250×21.2 mm) (flow 30 ml/min, λ=290nm) with water, 0.05% TFA (solvent A) and pure acetonitrile (solvent B) with the following gradient:
0.0 min→95% A; 14.8 min→50% A; 15.0 min→0% A; 18.0 min→0% A; 18.5 min→95% A, 22.0 min→95% A.

MS (ESI+) [MH]⁺ found: 1346.7 calc.: 1346.47 (C₆₁H₈₁N₁₄O₁₉S)

[M+Na]⁺ found: 1368.5 calc.: 1368.45 (C₆₁H₈₀N₁₄NaO₁₉S)

### Synthesis of Linker Toxin XVIa

### Variant a)

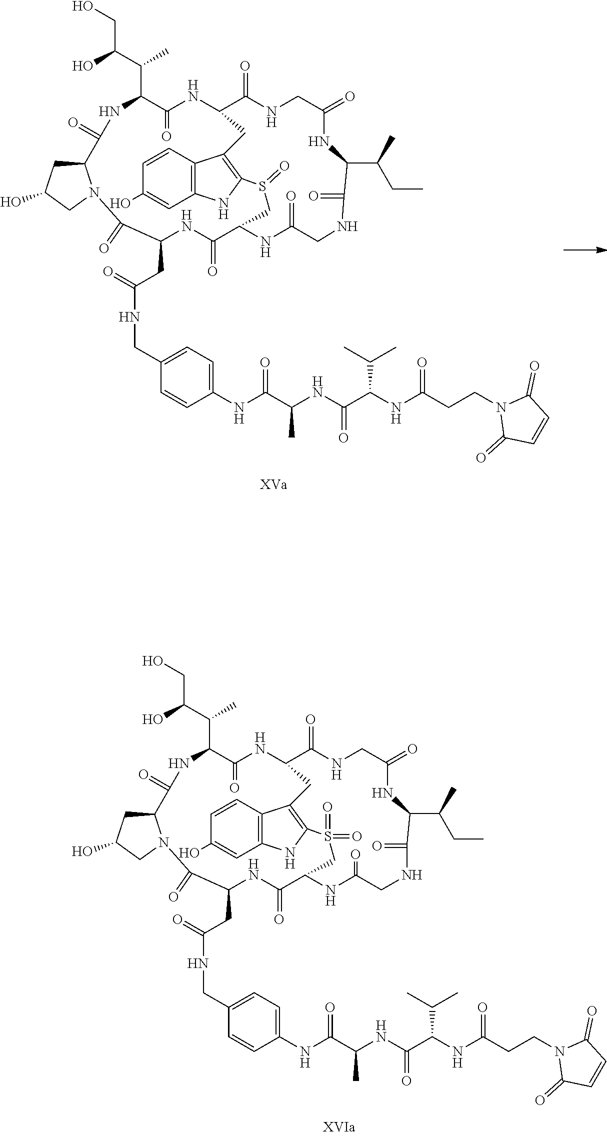 [SMILES: CC[C@H](C)[C@@H]1NC(=O)CNC(=O)[C@@H]2Cc3c([nH]c4cc(O)ccc34)S(=O)C[C@H](NC(=O)CNC1=O)C(=O)N[C@@H](CC(=O)NCc1ccc(NC(=O)[C@H](C)NC(=O)[C@@H](NC(=O)CCN3C(=O)C=CC3=O)C(C)C)cc1)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4cc(O)ccc34)S(=O)(=O)C[C@H](NC(=O)CNC1=O)C(=O)N[C@@H](CC(=O)NCc1ccc(NC(=O)[C@H](C)NC(=O)[C@@H](NC(=O)CCN3C(=O)C=CC3=O)C(C)C)cc1)C(=O)N1C[C@H](O)C[C@H]1C(=O)N[C@@H]([C@@H](C)[C@@H](O)CO)C(=O)N2]

10.09 mg (7.50 μmol) Sulfoxide XVa (as described in WO2016/142049) were dissolved in 2000 μl acetic acid. At ambient temperature 1784.28 μl of a solution of m-CPBA (meta-chloroperbenzoic acid, 69.5%, Aldrich) in acetic acid were added (stock solution: 5.18 mg m-CPBA in 5000 μl acetic acid). After stirring for 3.5 h at ambient temperature the solution was dropped into 30 ml ice-cold MTBE. The whole mixture was stored for additional 45 min at −18° C. and the precipitate was isolated by centrifugation. The solid residue was washed with 30 ml ice-cold MTBE and dried. The crude product was purified by HPLC to yield 5.82 mg (57%) of the pure product. The product was freeze-dried from acetonitrile/water (1/1) to a white amorphous solid.

HPLC purification:

Prep. HPLC was carried out with a Phenomenex Luna-C18 (2), 10 μm column (250×21.2 mm) (flow 30 ml/min, λ=305 nm) with water, 0.05% TFA (solvent A) and pure acetonitrile (solvent B) with the following gradient:
0.0 min→95% A; 14.8 min→50% A; 15.0 min→0% A; 18.0 min→0% A; 18.5 min→95% A, 22.0 min→95% A.

MS (ESI+) [MH]⁺ found: 1362.4 calc.: 1362.47 (C₆₁H₈₁N₁₄O₂₀S)

[M+Na]⁺ found: 1384.6 calc.: 1384.45 (C₆₁H₈₀N₁₄NaO₂₀S)

### Variant b)

### Step 1

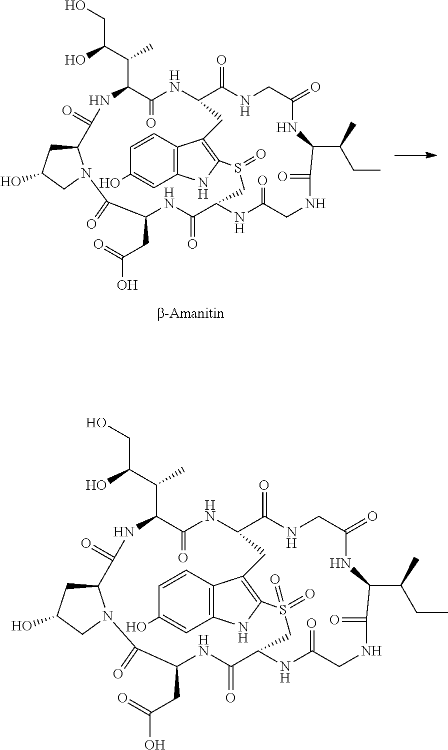 [SMILES: CC[C@H](C)[C@@H]1NC(=O)CNC(=O)[C@@H]2Cc3c([nH]c4cc(O)ccc34)S(=O)C[C@H](NC(=O)CNC1=O)C(=O)N[C@@H](CC(=O)O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4cc(O)ccc34)S(=O)(=O)C[C@H](NC(=O)CNC1=O)C(=O)N[C@@H](CC(=O)O)C(=O)N1C[C@H](O)C[C@H]1C(=O)N[C@@H]([C@@H](C)[C@@H](O)CO)C(=O)N2]

20.25 mg (22.01 μmol) β-Amanitin were dissolved in 4000 μl acetic acid. At ambient temperature 5634.38 μl of a solution of m-CPBA (meta-chloroperbenzoic acid, 69.5%, Aldrich) in acetic acid were added (stock solution: 10.67 mg m-CPBA in 10 ml acetic acid). After stirring for 3.5 h at ambient temperature the solution was dropped into 60 ml ice-cold MTBE. The whole mixture was stored for additional 30 min at −18° C. and the precipitate was isolated by centrifugation. The solid residue was washed with 60 ml ice-cold MTBE and dried. The crude product was purified by HPLC to yield 10.46 mg (51%) of the pure product. The product was freeze-dried from acetonitrile/water (1/1) to a white amorphous solid.

HPLC purification:

Prep. HPLC was carried out with a Phenomenex Luna-C18 (2), 10 μm column (250×21.2 mm) (flow 30 ml/min, λ=305 nm) with water, 0.05% TFA (solvent A) and pure acetonitrile (solvent B) with the following gradient:
0.0 min→97% A; 1.0 min→90% A; 23.0 min→86% A; 23.1 min→0% A; 27.0 min→0% A, 27.5 min→97% A; 30.0 min→97% A.

MS (ESI+) [MH]⁺ found: 936.9; calc.: 936.98 (C₃₉H₅₄N₉O₁₆S)

[M+Na]⁺ found: 958.9; calc.: 958.96 (C₃₉H₅₃N₉NaO₁₆S)

### Step 2

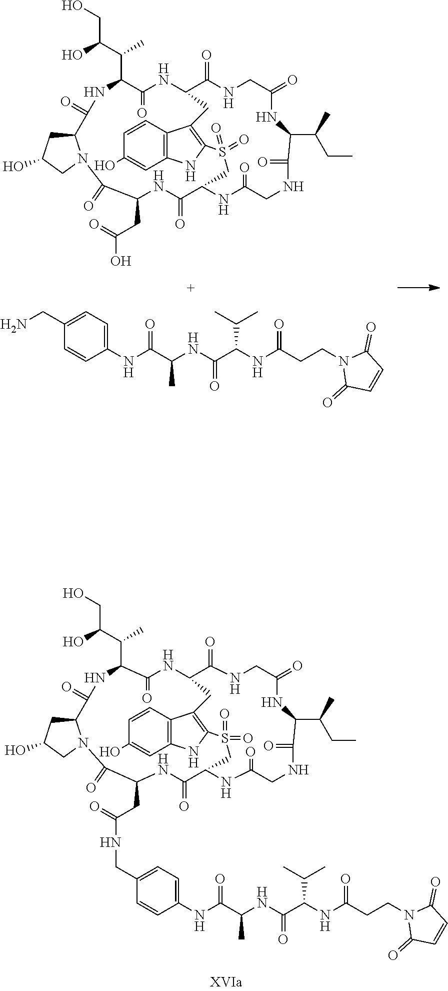 [SMILES: CC[C@H](C)[C@@H]1NC(=O)CNC(=O)[C@@H]2Cc3c([nH]c4cc(O)ccc34)S(=O)(=O)C[C@H](NC(=O)CNC1=O)C(=O)N[C@@H](CC(=O)O)C(=O)N1C[C@H](O)C[C@H]1C(=O)N[C@@H]([C@@H](C)[C@@H](O)CO)C(=O)N2 ; CC(C)[C@H](NC(=O)CCN1C(=O)C=CC1=O)C(=O)N[C@@H](C)C(=O)Nc1ccc(CN)cc1 ; CC[C@H](C)[C@@H]1NC(=O)CNC(=O)[C@@H]2Cc3c([nH]c4cc(O)ccc34)S(=O)(=O)C[C@H](NC(=O)CNC1=O)C(=O)N[C@@H](CC(=O)NCc1ccc(NC(=O)[C@H](C)NC(=O)[C@@H](NC(=O)CCN3C(=O)C=CC3=O)C(C)C)cc1)C(=O)N1C[C@H](O)C[C@H]1C(=O)N[C@@H]([C@@H](C)[C@@H](O)CO)C(=O)N2]

7.99 mg (8.54 μmol) of step 1 product were dissolved in 665 μl dry DMF. At ambient temperature 127.98 μl of a solution of TBTU in dry DMF were added. Afterwards a solution of 128.04 μl of DIPEA in dry DMF was added (stock solution: 52.25 μl DIPEA in 1500 μl dry DMF). The resulted solution was stirred for 1minute at ambient temperature under argon. Then a solution of 128.05 μl of BMP-Val-Ala-PAB-NH2 (WO2018115466) was added. The resulted reaction mixture was stirred for 2 h at room temperature. The solvent was removed under reduced pressure. The crude product was purified by HPLC to yield 7.34 mg (63%) of the pure product.

HPLC purification:

Prep. HPLC was carried out with a Phenomenex Luna-C18 (2), 10 μm column (250×21.2 mm) (flow 15 ml/min, λ=305 nm) 95% water, 5% methanol, 0.05% TFA (solvent A) and 95% methanol, 5% water, 0.05% TFA (solvent B) with the following gradient:
0.0 min→100% A; 5.0 min→50% A; 10.0 min→30% A; 20.0 min→20% A; 45.0 min→0% A, 50.0 min→100% A; 55.0 min→100% A.

### Synthesis of Linker Toxin XIXa

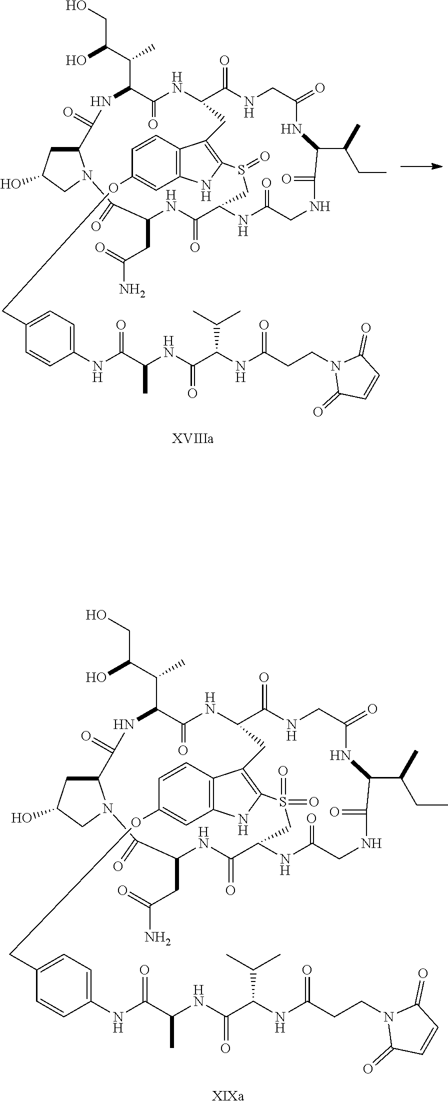

5.07 mg (3.77 μmol) Sulfoxide XVIIIa (as described in WO2016/142049) were dissolved in 1000 μl acetic acid. At ambient temperature 1043.57 μl of a solution of m-CPBA (meta-chloroperbenzoic acid, 69.5%, Aldrich) in acetic acid were added (stock solution: 4.93 mg m-CPBA in 5000 μl acetic acid). After stirring for 3 h 15 min at ambient temperature the solution was dropped into 15 ml ice-cold MTBE. The whole mixture was stored for additional 1 h at −18° C. and the precipitate was isolated by centrifugation. The solid residue was washed with 15 ml ice-cold MTBE and dried. The crude product was purified by HPLC to yield 2.44 mg (48%) of the pure product. The product was freeze-dried from acetonitrile/water (1/1) to a white amorphous solid.

HPLC purification:

Prep. HPLC was carried out with a Phenomenex Luna-C18 (2), 10 μm column (250×21.2 mm) (flow 30 ml/min, λ=305 nm) with water, 0.05% TFA (solvent A) and pure acetonitrile (solvent B) with the following gradient:
0.0 min→95% A; 14.8 min→50% A; 15.0 min→0% A; 18.0 min→0% A; 18.5 min→95% A, 22.0 min→95% A.

MS (ESI+) [MH]⁺ found: 1362.4; calc.: 1362.47 (C₆₁H₈₁N₁₄O₂₀S)

[M+Na]⁺ found: 1384.6; calc.: 1384.45 (C₆₁H₈₀N₁₄NaO₂₀S)

### Synthesis of Linker Toxin XXIIa

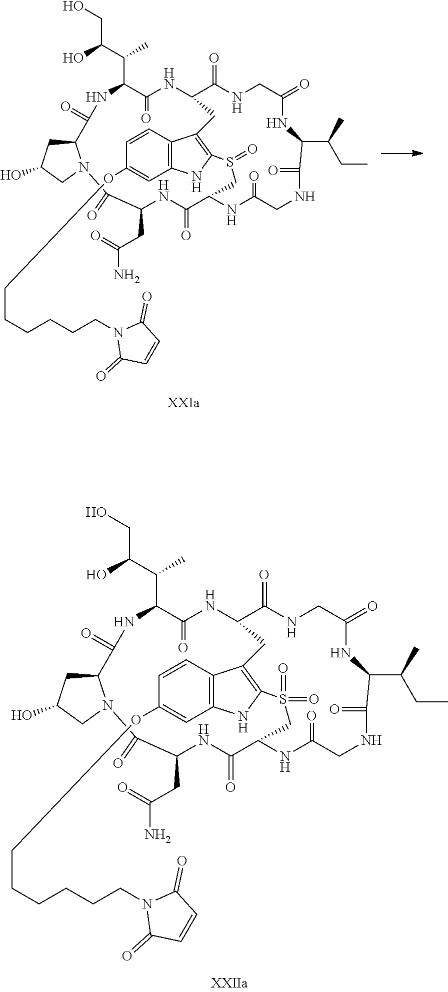 [SMILES: CC[C@H](C)[C@@H]1NC(=O)CNC(=O)[C@@H]2Cc3c([nH]c4cc(OCCCCCCN5C(=O)C=CC5=O)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4cc(OCCCCCCN5C(=O)C=CC5=O)ccc34)S(=O)(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

5.13 mg (4.67 μmol) Sulfoxide XXIa (WO2016142049) were dissolved in 1000 μl acetic acid. At ambient temperature 1291.27 μl of a solution of m-CPBA (meta-chloroperbenzoic acid, 69.5%, Aldrich) in acetic acid were added (stock solution: 4.94 mg m-CPBA in 5000 μl acetic acid). After stirring for 3.5 h at ambient temperature the solution was dropped into 15 ml ice-cold MTBE. The whole mixture was stored for additional 30 min on ice and the precipitate was isolated by centrifugation. The solid residue was washed with 15 ml ice-cold MTBE and dried. The crude product was purified by HPLC to yield 3.6 mg (69%) of the pure product. The product was freeze-dried from acetonitrile/water (1/1) to a white amorphous solid.

HPLC purification:

Prep. HPLC was carried out with a Phenomenex Luna-C18 (2), 10 μm column (250×21.2 mm) (flow 30 ml/min, λ=305 nm) with water, 0.05% TFA (solvent A) and pure acetonitrile (solvent B) with the following gradient:
0.0 min→95% A; 14.8 min→50% A; 15.0 min→0% A; 18.0 min→0% A; 18.5 min→95% A, 22.0 min→95% A.

MS (ESI+) [MH]⁺ found: 1115.4 calc.: 1115.22 (C₄₉H₆₈N₁₁O₁₇S)

[M+Na]⁺ found: 1137.2 calc.: 1137.20 (C₄₉H₆₇N₁₁NaO₁₇S)

### Example 2: Antibody-Targeted Amatoxin Conjugates

Antibodies were conjugated to the amatoxin linker conjugates by means of the so-called Thiomab technology. In this approach, the conjugation takes place by conjugation of the maleimide residue of the toxin linker construct to the free SH group of a cysteine residue in the antibody, as shown in the following reaction scheme:

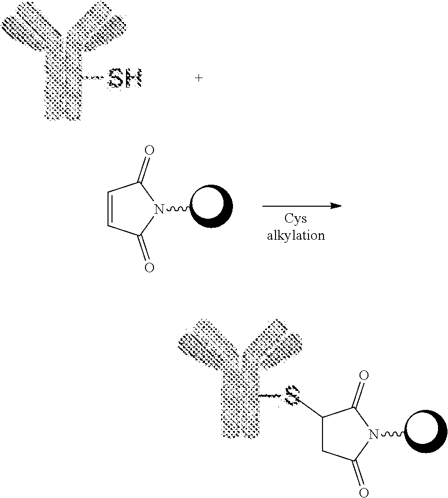

The principles of this conjugation method, are disclosed in Junutula et al (2008), the content of which is incorporated herein by reference.

The antibodies used in the present experiments comprise a D265C substitution in both Fc domains, in order to provide a cystein residue that has such free SH group. The respective technology is disclosed in WO2016/142049 A1, the content of which is incorporated herein by reference, and which results in a homogenous product with a fixed drug to antibody ration (“DAR”) of 2 and a site specific conjugation.

### Example 3: Induction of Immunogenic Cell Death by Antibody-Targeted Amatoxin Conjugates

### Example 3.1: Treatment of cells for ICD marker measurements

The Her2-positive cell line BT474 is a human breast cancer cell line. The CD79b-positive cell line BJAB is a human Burkitt lymphoma-derived B cell line.

Cells of the Her2-positive cell line BT474 and of the CD79b-positive cell line BJAB, respectively, were plated at 8×10⁴ cells per well in 100 μL of media. Flat bottom or U-bottom plates were used for BT474 or BJAB cells, respectively. The following day, media was changed. This step significantly improved ATP assay reproducibility by reducing variability among replicate wells. Then, cells were treated with media alone, Maytansine (100 nM), Amanitin (100 nM), Anti-HER2-Amanitin conjugate (50 nM antibody), or Anti-CD79b-Amanitin conjugate (50 nM antibody) for 16-72 h.

### Example 3.2: Calreticulin Measurement

Cells were assayed at 24, 48, and 72 h post-treatment. The adherent BT474 cells were lifted with a solution of highly purified, recombinant cell-dissociation enzymes (TrypLE, Thermo-Fisher) for 2 min. at 37° C. Both BT474 and BJAB cells were washed with PBS/2% FBS and then fixed in 0.5% paraformaldehyde for 5 min. After washing twice with cold PBS/2% FBS, cells were incubated for 5 min. with an Fc receptor binding inhibitor (FC block, eBioscience), blocking Fc receptor-mediated non-specific binding, followed by 30-min. incubation with Anti-Calreticulin-FITC (Abcam) or isotype control-FITC (Abcam). Cells were washed twice with PBS/2% FBS. Propidium iodide (PI) was added, and samples were analyzed by flow cytometry on a FACSCanto instrument (Becton-Dickinson) using FACS Diva software.

### Example 3.3: Extracellular ATP Measurement

At 16, 40, and 64 h post-treatment, culture media was transferred to reaction tubes (Eppendorf) and centrifuged gently. Extracellular ATP concentrations were determined by use of the ENLITEN ATP Assay (Promega). Chemiluminescence was measured on a SpectraMax ME chemiluminescence reader (Molecular Devices).

### Example 3.4: Extracellular HMGB1 Measurement

Culture media was collected at 24, 48, and 72 h post-treatment. Analytes were captured on a Nunc Maxisorp 96-well plate coated at pH 9 with 1 μg/mL Anti-HMGB1 antibody (clone 1D5, Sigma), and plates were blocked with casein buffer (ThermoFisher). A standard curve was generated using recombinant human HMGB1 protein (R&D Systems) added to culture media that had been incubated at 37° C./5% CO₂ for the same number of days as the experimental samples. This step had the effect of normalizing background signals observed in fresh culture media. Media from the experimental samples were transferred to fresh tubes, centrifuged gently to pellet debris, and then added to the prepared plate along with the standard curve. After 1 h, the plate was washed with PBS/0.1% Tween-20, and an Anti-HMGB1 polyclonal antibody (ab18256, Abcam) was added at 1 μg/mL in PBS for 1 h. The plate was washed and an anti-rabbit peroxidase-conjugated secondary antibody (Jackson Immunoresearch) was applied at a 1:3000 dilution in PBS. After 30 min, the plate was washed with PBS/0.1% Tween-20, and bound secondary antibody was detected using Ultra TMB (Thermo Fisher); signals were read on a Molecular Devices Spectra Max M5 plate reader. With respect to sample handling, experimental samples could not be frozen for subsequent analysis as the HMGB1 signal degraded. However, samples could be kept at 4° C. for analysis on the following day.

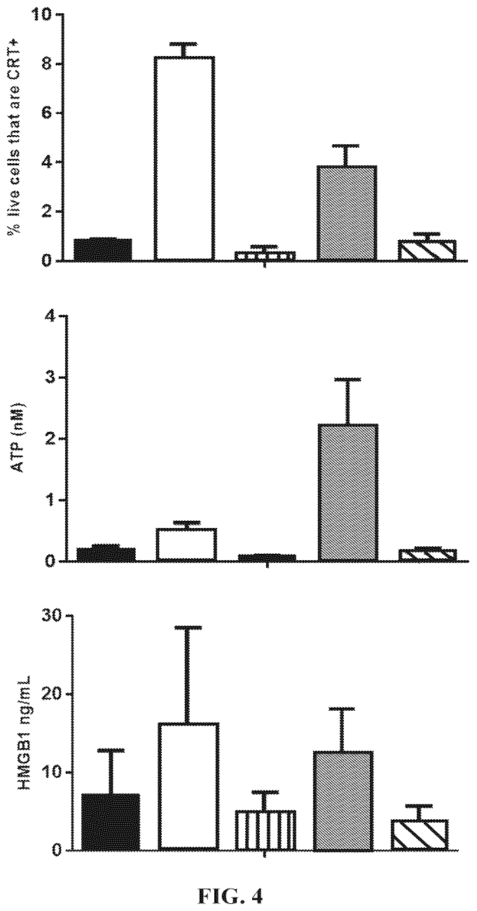

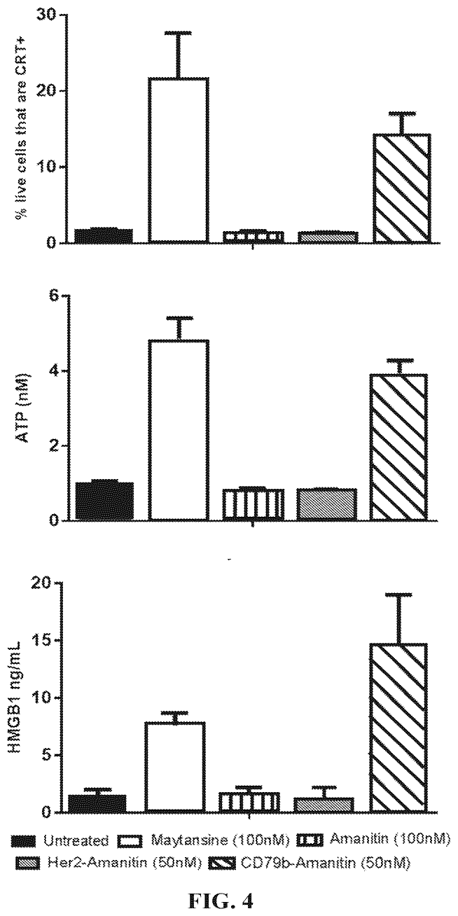

Results of ICD marker measurements from BT474 and BJAB cells, respectively, are shown in FIG. 4.

In contrast to unconjugated Maytansine, unconjugated Amanitin did not induce cell surface exposure of calreticulin (CRT), ATP secretion, or HMGB1 release by either of the two cell lines, as compared to control cells treated with media only. This finding is consistent with the poor cellular uptake of unconjugated amanitin. However, amanitin-conjugated antibody-drug conjugates (ADCs) induced exposure and secretion of said ICD markers, respectively, in a target-dependent manner.

In Her2-positive BT474 cells (FIG. 4A-C), the Anti-HER2-Amanitin conjugate, but not the Anti-CD79b-Amanitin conjugate, induced cell surface exposure of CRT (FIG. 4A), ATP secretion (FIG. 4 B), and HMGB1 release (FIG. 4 C). In contrast, in CD79b-positive BJAB cells (FIG. 4 D-F), the Anti-CD79b-Amanitin conjugate, but not the Anti-HER2-Amanitin conjugate induced cell surface exposure of CRT (FIG. 4 D), ATP secretion (FIG. 4 E), and HMGB1 release (FIG. 4 F).

Hence, the ATAC-induced exposure of CRT, secretion of ATP, and release of HMGB1 in said cell lines was dependent from the specificity of the target-binding moiety of the ATAC.

### Example 4: Synergistic Cytotoxic Action of ATAC and Immune Checkpoint Inhibitor In Vivo

The cytotoxic activity of the combination comprising an ICI and an ATAC has been assessed by use of a tumor mouse model in vivo. The study consisted of 6 experimental groups with 12 animals each. CD19-positive Raji cells (human Burkitt lymphoma, DSMZ) were premixed with human peripheral blood mononuclear cells (PBMCs, German Red Cross) and inoculated subcutaneously on study day 0. Treatment with PBS, the CD19-specific ATAC chiBCE19-D265C-XIIb alone at a dose of 0.1 mg/kg body weight and a dose of 0.3 mg/kg body weight (single dose i.v.),, respectively, the PD-L1-specific antibody Avelumab alone at a dose of 20 mg/kg body weight (i.v. on days 0, 3, 6, 8, 10, 13), or a combination of the CD19-specific ATAC chiBCE19-D265C-XIIb at a dose of 0.1 mg/kg body weight and a dose of 0.3 mg/kg body weight (single dose i.v.), respectively, and the PD-L1-specific antibody Avelumab at a dose of 20 mg/kg body weight (i.v. on days 0, 3, 6, 8, 10, 13), was started on day 0 after cell inoculation. Tumor volumes were measured twice per week by calliper measurements and body weights were determined in parallel. Animals were sacrificed and a necropsy performed when either tumor volumes were >1600 mm³ or when mice needed to be sacrificed due to ethical reasons (according to German animal welfare legislation).

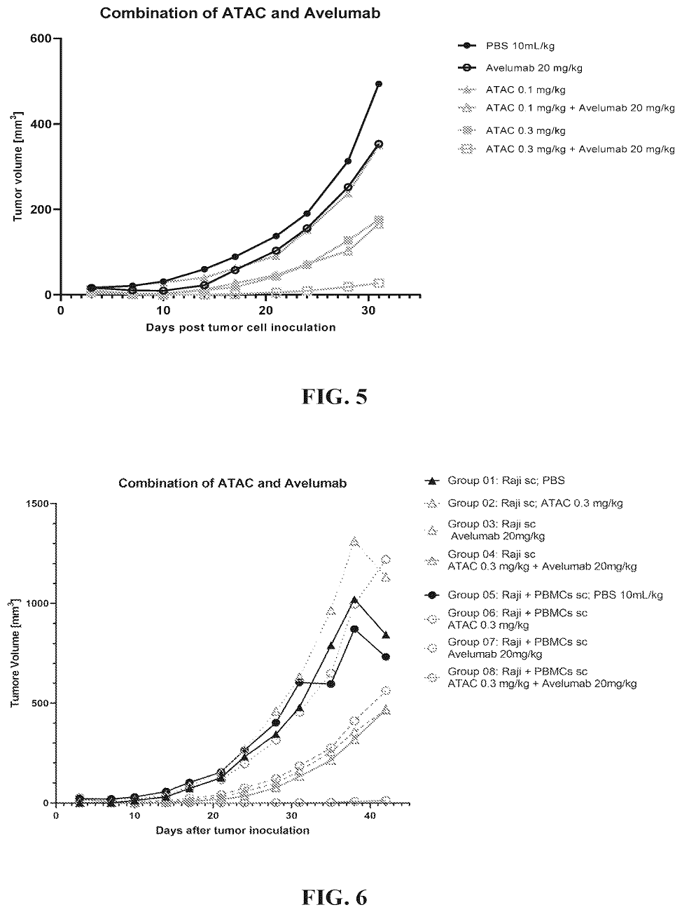

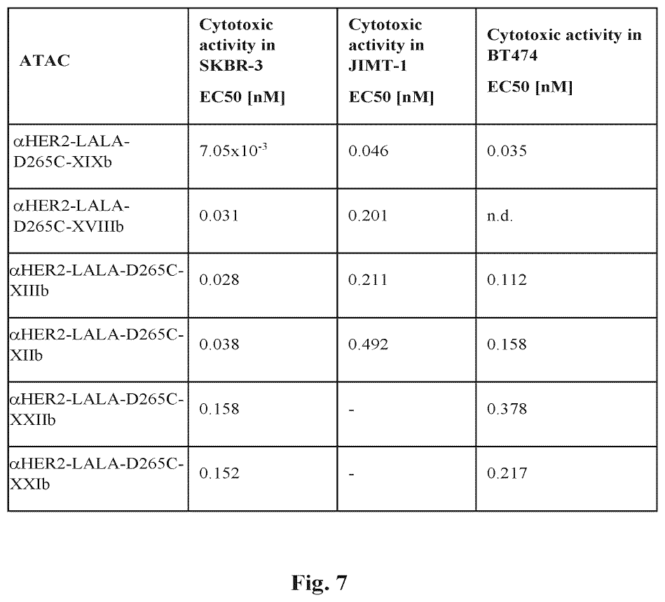

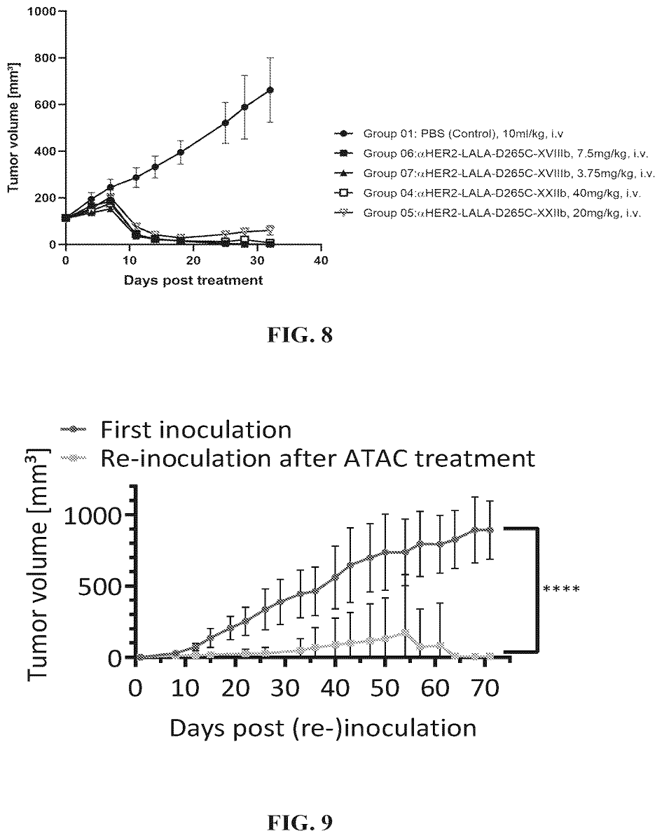

Results of the study are shown in FIG. 5. Reduction of the tumor volumes achieved by either the PD-L1-specific antibody Avelumab alone or the CD19-specific ATAC chiBCE19-D265C-XIIb alone at a dose of 0.1 mg/kg body weight, as compared to the control group (PBS), were comparable. However, reduction of tumor volumes found with the combination of the CD19-specific ATAC chiBCE19-D265C-XIIb at a dose of 0.1 mg/kg body weight and the PD-L1-specific antibody Avelumab was significantly higher than the sum of reductions achieved by both individual agents alone. The same was observed when the ATAC was used at a higher concentration of 0.3 mg/kg body weight indicating a synergistic effect of the ATAC and the Immune Checkpoint Inhibitor with regard to their tumor-cell killing activities in vivo.

TABLE 1
Mean Tumorvolumes at day 31 post tumor cell inoculation.

| | Mean TV | |
|---|---|---|
| | [mm³] | SEM |
| PBS 10 mL/kg | 493.6 | 106.1 |
| Avelumab 20 mg/kg | 353.1 | 76.3 |
| ATAC 0.1 mg/kg | 349.9 | 87.7 |
| ATAC 0.1 mg/kg + Avelumab 20 mg/kg | 165.5 | 26.9 |
| ATAC 0.3 mg/kg | 176.0 | 87.5 |
| ATAC 0.3 mg/kg + Avelumab 20 mg/kg | 27.4 | 22.1 |

TV: tumor volumes;
SEM: standard error of the mean

### Example 4: Dependency of the Synergistic Cytotoxic Action of ATAC and Immune Checkpoint Inhibitor In Vivo from the Presence of PBMCs

The cytotoxic activity of the combination comprising an ICI and an ATAC has further been assessed by use of a tumor mouse model in vivo in the absence and presence of human peripheral blood mononuclear cells (PBMCs). The study consisted of 8 experimental groups with 12 animals each. For 4 groups, CD19-positive Raji cells (human Burkitt lymphoma, DSMZ) were premixed with human PBMCs (German Red Cross) and inoculated subcutaneously on day 0. The remaining 4 groups were inoculated subcutaneously with Raji cells only. Treatment with PBS, the CD19-specific ATAC chiBCE19-D265C-XIIb alone at a dose of 0.3 mg/kg body weight, or the PD-L1-specific antibody Avelumab (Bavencio®) alone at a dose of 20 mg/kg body weight, or the combination of the CD19-specific ATAC chiBCE19-D265C-XIIb and the PD-L1-specific antibody Avelumab, was started on day 0 after cell inoculation (for details see Table 2), whereby Avelumab and the CD19-specific ATAC chiBCE19-D265C-XIIb were administered i.v. sequentially. Tumor volumes were measured twice per week by calliper measurements, and body weights were determined in parallel. Animals were sacrificed and necropsies were performed when either tumor volumes were >1600 mm³ or when the mice needed to be sacrificed due to ethical reasons (according to German animal welfare legislation).

TABLE 2
Experimental Details of the Study in Example 4

| | | Dose | | Inoculated |
|---|---|---|---|---|
| | | Protein | | cells s.c. |
| Group | Treatment | [mg/kg] | Schedule | (day 0) |
| 1 | PBS | 10 ml/kg | Day 0, 3, 6, | Raji |
| | | | 8, 10, 13 | |
| 2 | chiBCE19- | 0.3 | Single dose, | Raji |
| | D265C-XIIb | | day 0 | |
| 3 | Avelumab | 20 | Day 0, 3, 6, | Raji |
| | | | 8, 10, 13 | |
| 4 | chiBCE19- | 0.3 | Single dose, | Raji |
| | D265C-XIIb | | day 0 | |
| | Avelumab | 20 | Day 0, 3, 6, | |
| | | | 8, 10, 13 | |
| 5 | PBS | 10 ml/kg | Day 0, 3, 6, | Raji + PBMC Donor 1 |
| | | | 8, 10, 13 | Raji + PBMC Donor 2 |
| | | | | Raji + PBMC Donor 3 |
| | | | | Raji + PBMC Donor 4 |
| 6 | chiBCE19- | 0.3 | Single dose, | Raji + PBMC Donor 1 |
| | D265C-XIIb | | day 0 | Raji + PBMC Donor 2 |
| | | | | Raji + PBMC Donor 3 |
| | | | | Raji + PBMC Donor 4 |
| 7 | Avelumab | 20 | Day 0, 3, 6, | Raji + PBMC Donor 1 |
| | | | 8, 10, 13 | Raji + PBMC Donor 2 |
| | | | | Raji + PBMC Donor 3 |
| | | | | Raji + PBMC Donor 4 |
| 8 | chiBCE19- | 0.3 | Single dose, | Raji + PBMC Donor 1 |
| | D265C-XIIb | | day 0 | Raji + PBMC Donor 2 |
| | Avelumab | 20 | Day 0, 3, 6, | Raji + PBMC Donor 3 |
| | | | 8, 10, 13 | Raji + PBMC Donor 4 |

Results of the study are shown in FIG. 6. In the absence of human PBMCs, the combination of the CD19-specific ATAC chiBCE19-D265C-XIIb at a dose of 0.3 mg/kg body weight with the PD-L1-specific antibody Avelumab at a dose of 20 mg/kg body weight did not result in a higher reduction in tumor volumes in vivo than achieved by the CD19-specific ATAC chiBCE19-D265C-XIIb alone. In contrast, in the presence of human PBMCs, the combination of the CD19-specific ATAC chiBCE19-D265C-XIIb at a dose of 0.3 mg/kg body weight with the PD-L1-specific antibody Avelumab at a dose of 20 mg/kg body weight yielded a higher reduction in tumor volumes in vivo as compared to the reduction achieved by the CD19-specific ATAC chiBCE19-D265C-XIIb alone.

In conclusion, the synergistic cytotoxic effect of ATAC and Immune Checkpoint Inhibitor in vivo depends on the presence of human PBMCs in the mouse model used.

### Example 5: Efficacy of Amanitin Based anti-CD19 ATAC chiBCE19-D265C-XIIb and Pembrolizumab in a Raji Tumor Xenograft Model in NOD/SCID Mice Reconstituted with Human PBMCs

TABLE 3
Experimental Details of the Study of Example 5

| | | | | Inoculated |
|---|---|---|---|---|
| | | dose | | cells s.c. |
| Group | Treatment | [mg/kg] | Schedule | (d 0) |
| 1 | 0.9% NaCl | 10 mL//kg | Day 0, 3, 6, 8, 10 | Raji + PBMC |
| | | | | Raji + PBMC |
| | | | | Raji + PBMC |
| 2 | chiBCE19- | 0.1 | Single dose, day0 | Raji + PBMC |
| | D265C-XIIb | | | Raji + PBMC |
| | | | | Raji + PBMC |
| 3 | chiBCE19- | 0.3 | Single dose, day0 | Raji + PBMC |
| | D265C-XIIb | | | Raji + PBMC |
| | | | | Raji + PBMC |
| 4 | Pembrolizumab | 20 | Day 0, 3, 6, 8, 10 | Raji + PBMC |
| | | | | Raji + PBMC |
| | | | | Raji + PBMC |
| 5 | chiBCE19- | 0.1 | Single dose, day0 | Raji + PBMC |
| | D265C-XIIb | | | Raji + PBMC |
| | Pembrolizumab* | 20 | Day 0, 3, 6, 8, 10 | Raji + PBMC |
| 6 | chiBCE19- | 0.3 | Single dose, day0 | Raji + PBMC |
| | D265C-XIIb | | | Raji + PBMC |
| | Pembrolizumab* | 20 | Day 0, 3, 6, 8, 10 | Raji + PBMC |

*dosing of chiBCE19-D265C-XIIb and Pembrolizumab was done sequentially on day 0

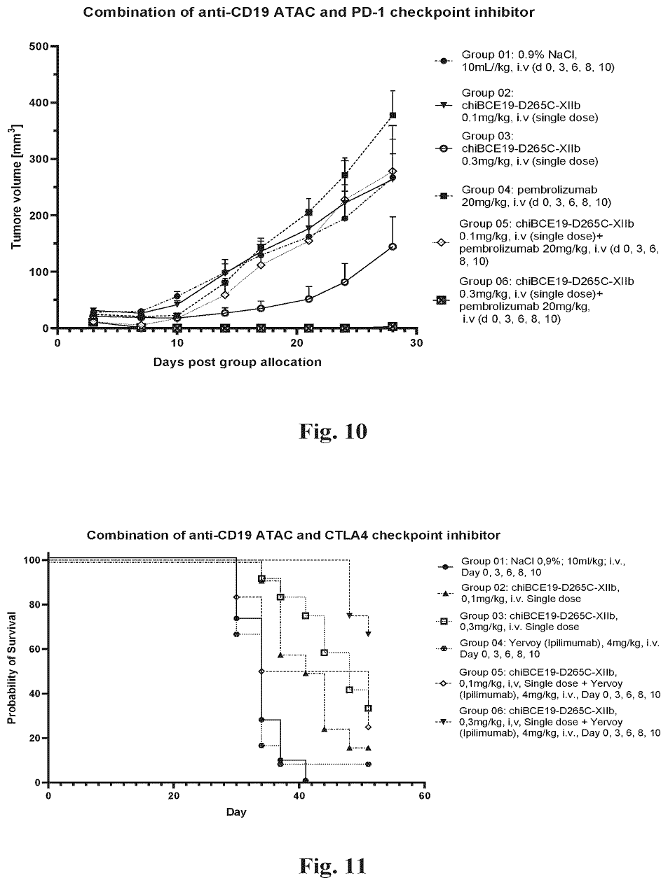

The study consisted of 6 experimental groups with 9 animals each. For all groups, CD19-positive Raji cells (human Burkitt lymphoma, DSMZ) were premixed with human PBMCs (German Red Cross) and inoculated subcutaneously on day 0. Treatment with PBS, the CD19-specific ATAC chiBCE19-D265C-XIIb alone at a dose of 0.1 mg/kg and 0.3 mg/kg body weight, or the PD-1-specific antibody Pembrolizumab (Keytruda®) alone at a dose of 20 mg/kg body weight, or the combination of the CD19-specific ATAC chiBCE19-D265C-XIIb and the PD-1-specific antibody Pembrolizumab, was started on day 0 after cell inoculation (for details see Table 3). Tumor volumes were measured twice per week by caliper measurements, and body weights were determined in parallel. Animals were sacrificed and necropsies were performed when either tumor volumes were >1600 mm³ or when the mice needed to be sacrificed due to ethical reasons (according to German animal welfare legislation). Results of the study are shown in FIG. 10 which illustrate that the synergistic cytotoxic effect of ATAC and anti-PD-1 Immune Checkpoint Inhibitor in vivo depends on the presence of human PBMCs in the mouse model used and that an effective dose for the anti-CD19 ATAC is needed in order to induce immunogenic cell death which synergizes with the activity of the immune checkpoint inhibitor.

### Example 6: Efficacy of anti-CD19 ATAC chiBCE19-D265C-XIIb and Ipilimumab in a Raji Tumor Xenograft Model in NOD/SCID Mice Reconstituted with Human PBMCs

TABLE 4
Experimental Details of the Study of Example 6, all animals
were inoculated with Raji + PBMC cells (s.c.) at day 0

| | | | Application | |
|---|---|---|---|---|
| | | dose | volume | |
| Group | Treatment | [mg/kg] | [mL/kg] | Schedule |
| 1 | 0.9% NaCl | 10 mL//kg | 10 | Day 0, 3, 6, 8, 10 |
| 2 | chiBCE19- | 0.1 | 10 | Single dose, day0 |
| | D265C-XIIb | | | |
| 3 | chiBCE19- | 0.3 | 10 | Single dose, day0 |
| | D265C-XIIb | | | |
| 4 | Yervoy | 4 | 10 | Day 0, 3, 6, 8, 10 |
| | (Ipilimumab) | | | |
| | chiBCE19- | 10 | 0.1 | Single dose, day0 |
| | D265C-XIIb | | | |
| 5 | Yervoy | 4 | 10 | Day 0, 3, 6, 8, 10 |
| | (Ipilimumab)* | | | |
| 6 | chiBCE19- | 0.3 | 10 | Single dose, day0 |
| | D265C-XIIb | | | |
| | Yervoy | 4 | 10 | Day 0, 3, 6, 8, 10 |
| | (Ipilimumab)* | | | |

*dosing of chiBCE19-D265C-XIIb and Ipilimumab was done sequentially on day 0

The study consisted of 6 experimental groups with 12 animals each. For all groups, CD19-positive Raji cells (human Burkitt lymphoma, DSMZ) were premixed with human PBMCs (German Red Cross) and inoculated subcutaneously on day 0. Treatment with PBS, the CD19-specific ATAC chiBCE19-D265C-XIIb alone at a dose of 0.1 mg/kg and 0.3 mg/kg body weight, or the CTLA4-specific antibody Ipilimumab (Yervoy®) alone at a dose of 4 mg/kg body weight, or the combination of the CD19-specific ATAC chiBCE19-D265C-XIIb and the CTLA4-specific antibody Ipilimumab, was started on day 0 after cell inoculation (for details see Table 4). Animals were sacrificed and necropsies were performed when either tumor volumes were >1600 mm³ or when the mice needed to be sacrificed due to ethical reasons (according to German animal welfare legislation). Results of the study are shown in FIG. 11 which illustrate a synergistic effect on survival in animals that received a combination treatment of CTLA4 immune checkpoint inhibitor and anti-CD19-ATAC (group 6). The results indicate that an effective amount of the anti-CD19 ATAC is needed to induce immunogenic cell death which in combination with a CTLA4 checkpoint inhibitor (e.g. Ipilimumab) acts synergistically as evidenced by prolonged survival of the respective treatment group.

### References

Darvin P et al. (2018). Immune checkpoint inhibitors: recent progress and potential biomarkers. Experimental & Molecular Medicine Vol. 50: 165-176.
Dyck L and Mills KHG (2017). Immune checkpoints and their inhibition in cancer and infectious diseases. Eur. J. Immunol. Vol. 47: 765-779.
Hemler ME (2001). Specific tetraspanin functions. The Journal of Cell Biology Vol. 155(7): 1103-1107.
Kroemer G et al. (2013). Immunogenic cell death in cancer therapy. Annu. Rev. Immunol. Vol. 31: 51-72.
Li et al. (2016). A mini-review for cancer immunotherapy: molecular understanding of PD-1/PD-L1 pathway and translational blockade of immune checkpoints. Int. J. Mol. Sci. Vol. 17:1151 (doi:10.3390/ijms17071151).
Marshall HT and Djamgoz MBA (2018). Immuno-oncology: Emerging targets and combination therapies. Front. Oncol. Vol. 8: 315 (doi: 10.3389/fonc.2018.00315)
Martins et al. (2019). Adverse effects of immune-checkpoint inhibitors: epidemiology, management and surveillance. Nature Reviews—Clinical Oncology Vol. 16: 563-580.
Obeid et al. (2007). Calreticulin exposure dictates the immunogenicity of cancer cell death. Nature Medicine Vol. 13: 54-61.
Qin et al. (2019). Novel immune checkpoint targets: moving beyond PD-1 and CTLA-4. Molecular Cancer Vol. 18: 155-168.
Sambi M et al. (2019). Current challenges in cancer immunotherapy: multimodal approaches to improve efficacy and patient response rates. Journal of Oncology Vol. 2019 (doi.org/10.1155/2019/4508794).
Singh S et al. (2020). Immune checkpoint inhibitors: a promising anticancer therapy. Drug Discovery Today Vol. 25 (1): 223-229.
Taams LS and de Gruijl TD (2020). Immune checkpoint inhibition: from molecules to clinical application. Clinical and Experimental Immunology Vol. 200: 105-107.
Wei SC et al. (2018). Fundamental mechanisms of immune checkpoint blockade therapy. Cancer Discov. Vol. 8(9): 1069-1086.
Wieland et al. (1978). Amatoxins, phallotoxins, phallolysin, and antamanide: the biologically active components of poisonous amanita mushroom. CRC Crit Rev Biochem. Vol. 5: 185-260.
Xu-Monette ZY et al. (2016). Assessment of CD37 B-cell antigen and cell of origin significantly improves risk prediction in diffuse large B-cell lymphoma. Blood Vol. 128(26): 3083-3100.
Zou F et al. (2018). Expression and Function of Tetraspanins and Their Interacting Partners in B Cells. Frontiers in Immunology Vol. 9. Article 1606.

What is claimed is:

1. A composition comprising
(a) at least one immune checkpoint inhibitor and
(b) at least one conjugate, wherein said conjugate comprises
(i) a target binding moiety,
(ii) at least one amatoxin, and
(iii) optionally at least one linker connecting said target binding moiety with said at least one amatoxin,
wherein said target binding moiety of said conjugate is an antibody having an Fc region comprising a D265C mutation (according to the EU numbering system),
wherein said linker, if present, or said amatoxin is connected to said antibody via the D265C residue of said antibody, and
wherein said immune checkpoint inhibitor is nivolumab, pidilizumab, pembrolizumab, atezolizuab, avelumab, durvalumab, cemiplimab, or ipilimumab, or an antigen-binding fragment thereof.

2. The composition of claim 1, wherein said target binding moiety of said conjugate is a murine, a chimeric, a humanized or a human antibody, or antigen-binding fragment thereof, respectively.

3. The composition of claim 1, wherein the target binding moiety of said conjugate binds to a target molecule on the cell surface of cancer cells selected from the group consisting of PSMA, CD19, CD37, CD269, sialyl Lewisª, HER-2/neu, and epithelial cell adhesion molecule (EpCAM).

4. The composition of claim 1, wherein the target binding moiety further comprises at least one mutation selected from the group consisting of D265A, A118C, L234A, and L235A.

5. The composition of claim 1, wherein the amatoxin of said conjugate is selected from the group consisting of α-amanitin, β-amanitin, γ-amanitin, ε-amanitin, amanin, amaninamide, amanullin, amanullinic acid, and salts and analogues thereof.

6. The composition of claim 1, wherein the linker of said conjugate, if present, is a non-cleavable or a cleavable linker, and wherein said cleavable linker is selected from the group consisting of an enzymatically cleavable linker and a chemically cleavable linker.

7. The composition of claim 1, wherein the linker of said conjugate, if present, or said target binding moiety is connected to said amatoxin via (i) the γ C-atom of amatoxin amino acid 1, or (ii) the δ C-atom of amatoxin amino acid 3, or (iii) the 6′-C-atom of amatoxin amino acid 4.

8. The composition of claim 1, wherein said conjugate comprises any of the following compounds of formulas (I) to (XII), respectively, as linker-amatoxin moieties:

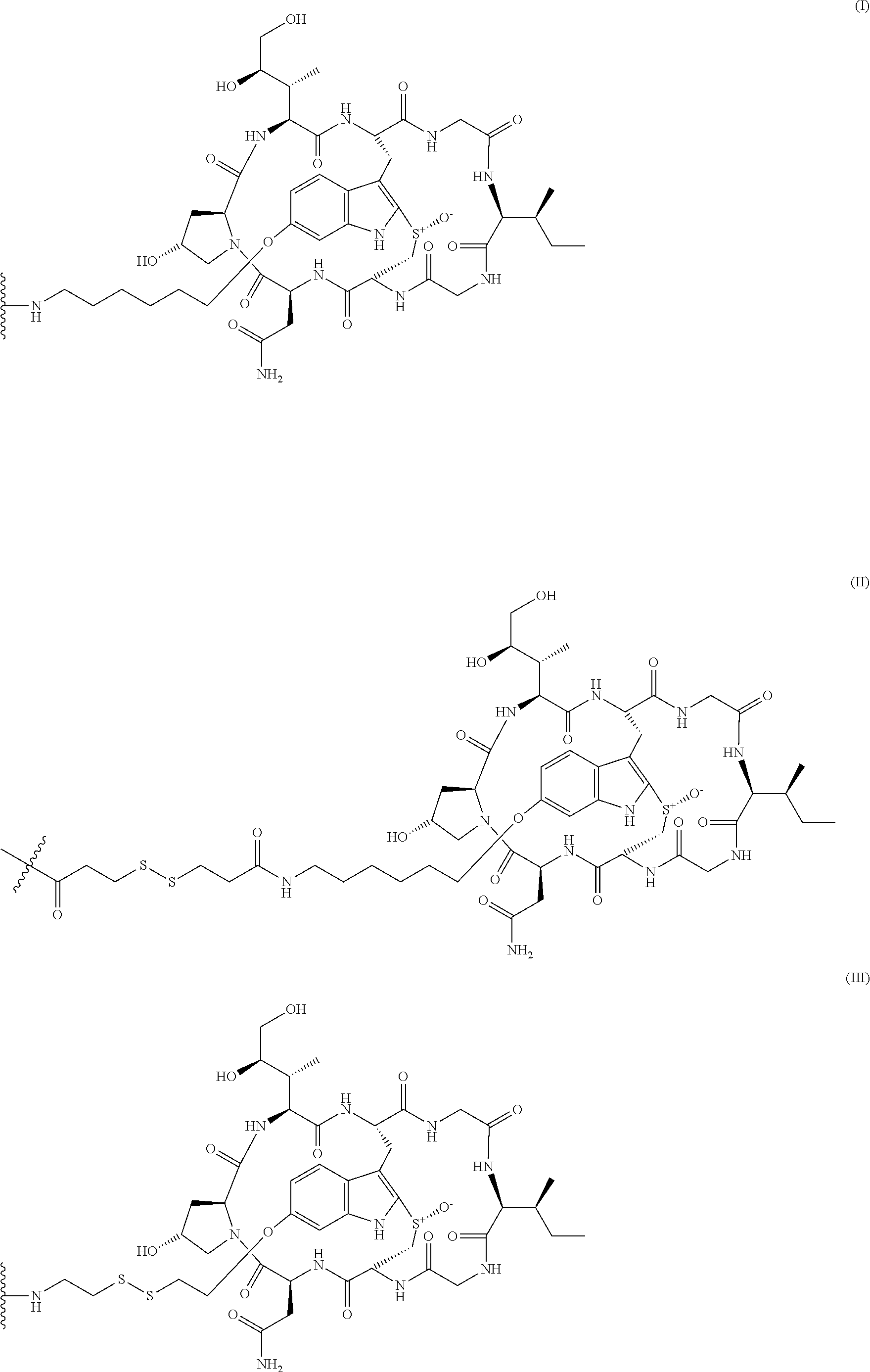

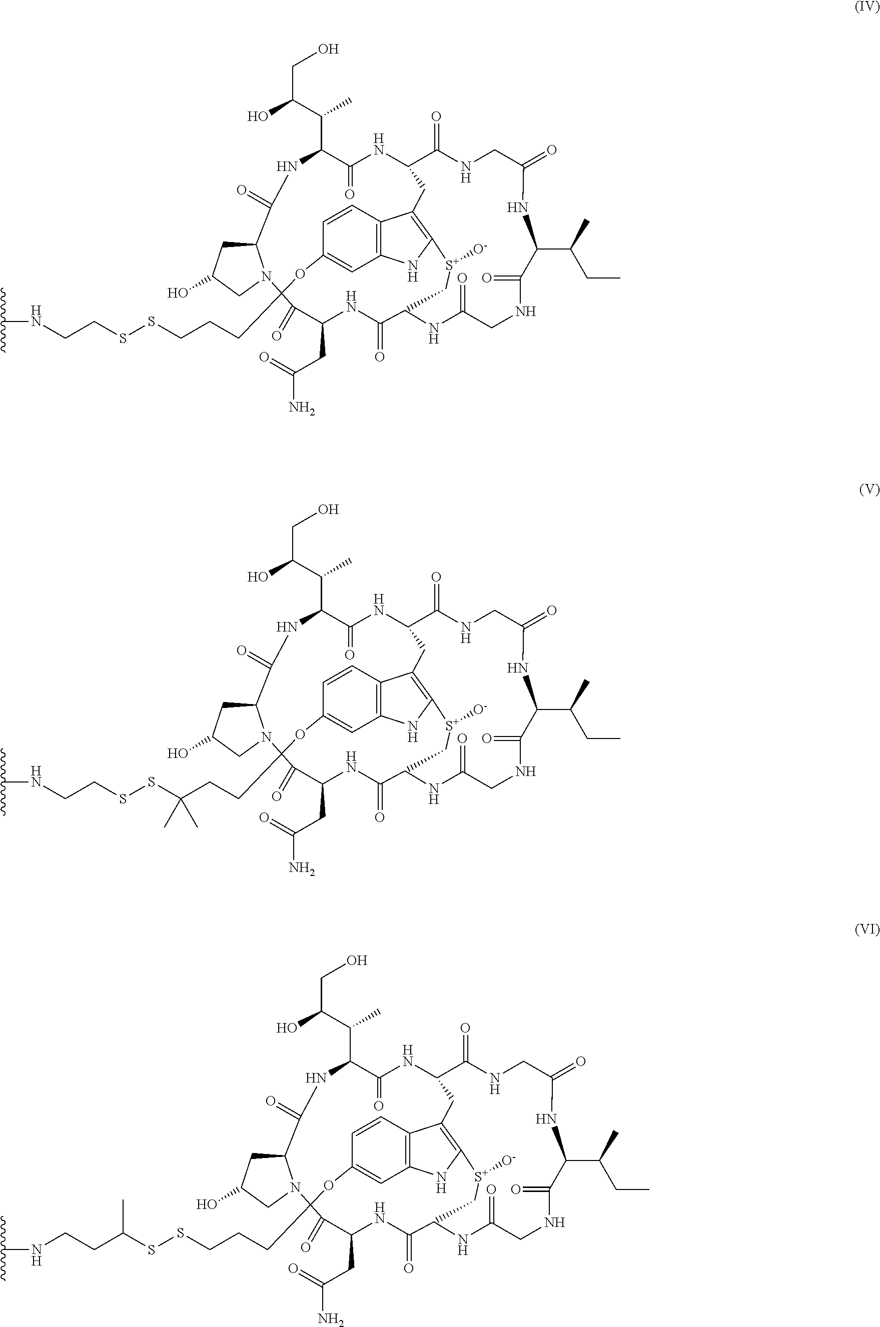

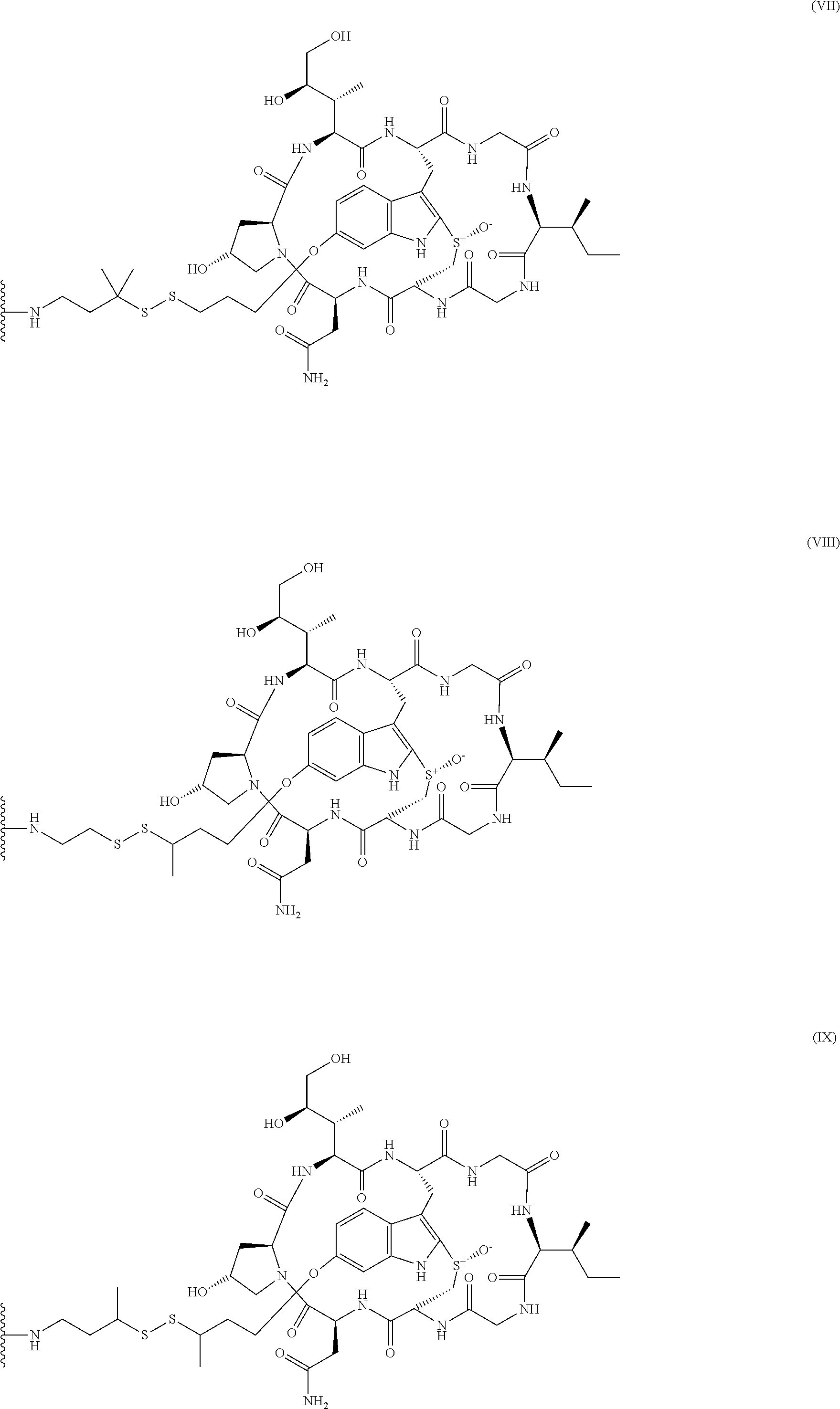

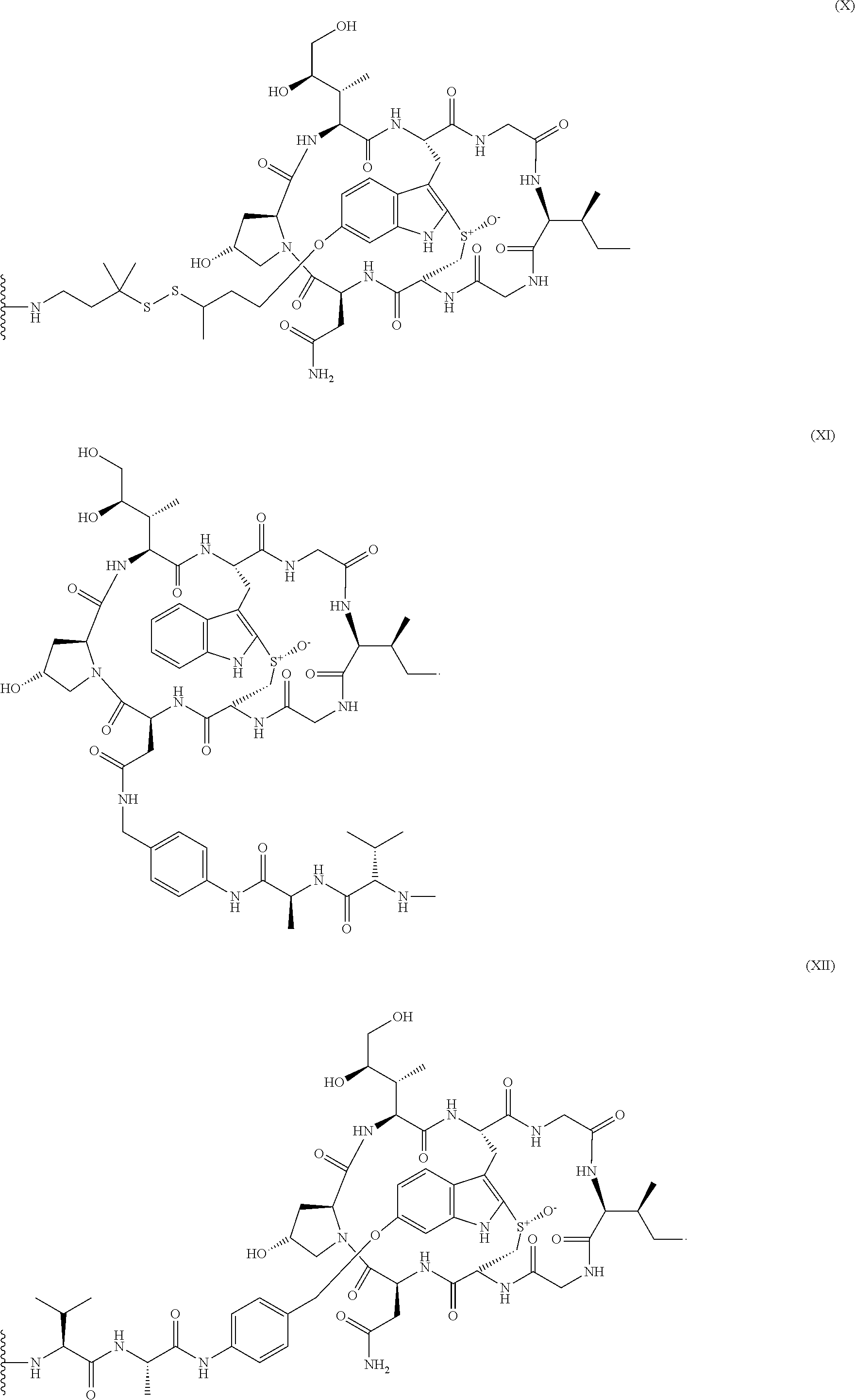

9. The composition of claim 1, wherein said conjugate is a compound according to any one of formulas XIII to XXII: 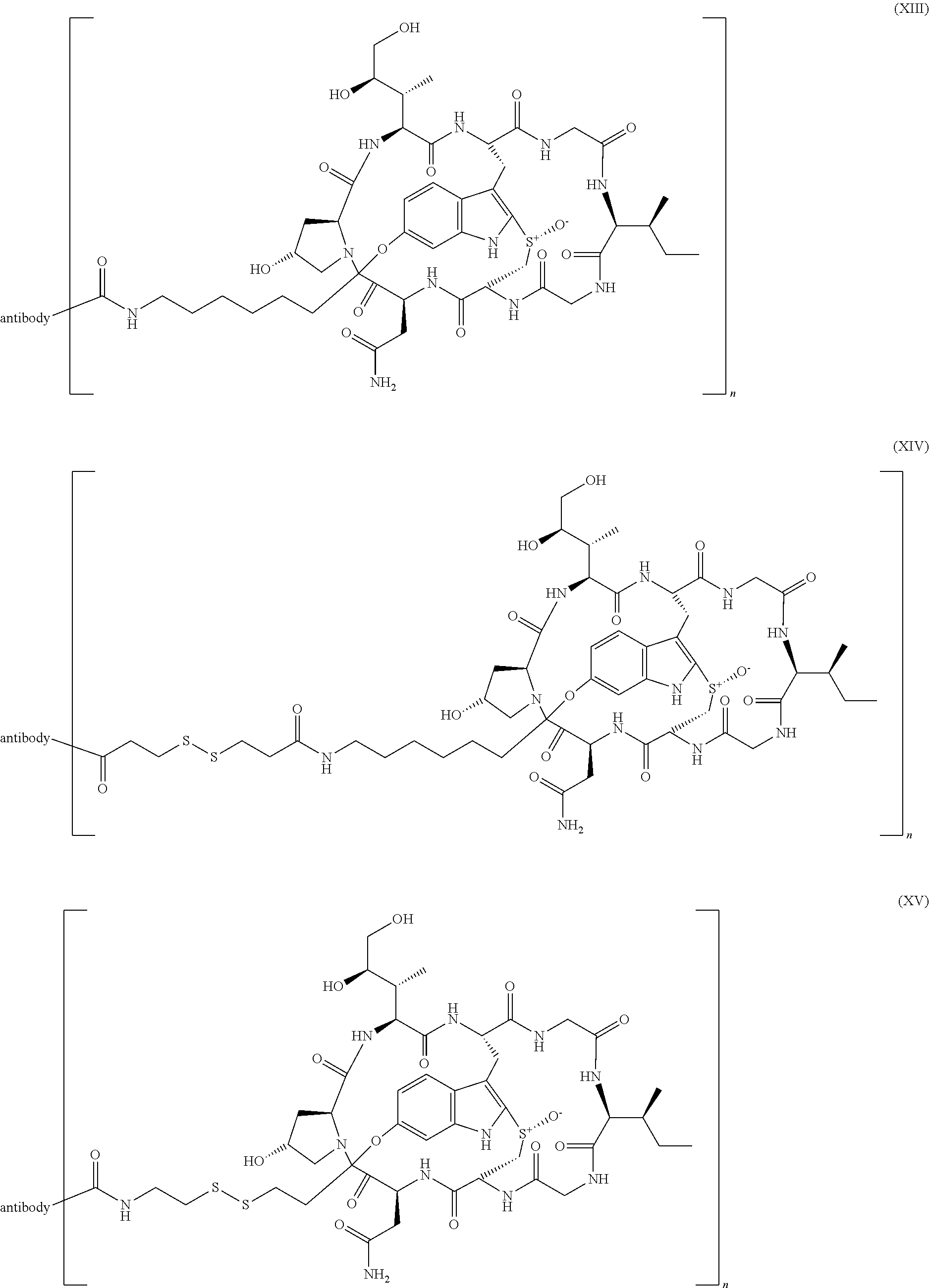 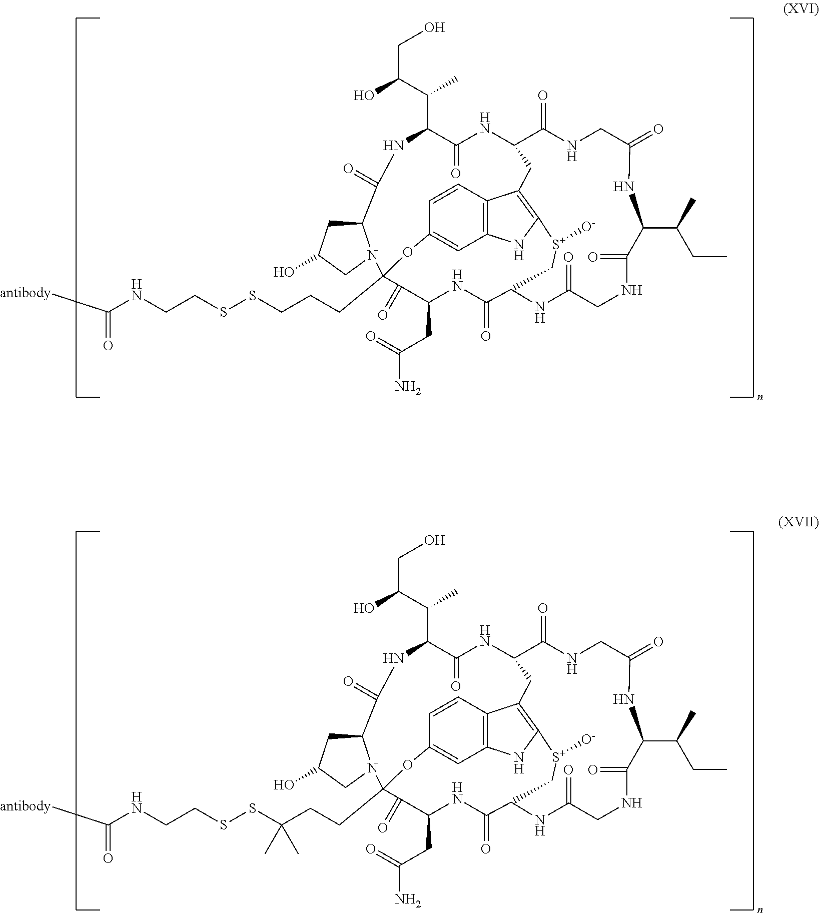 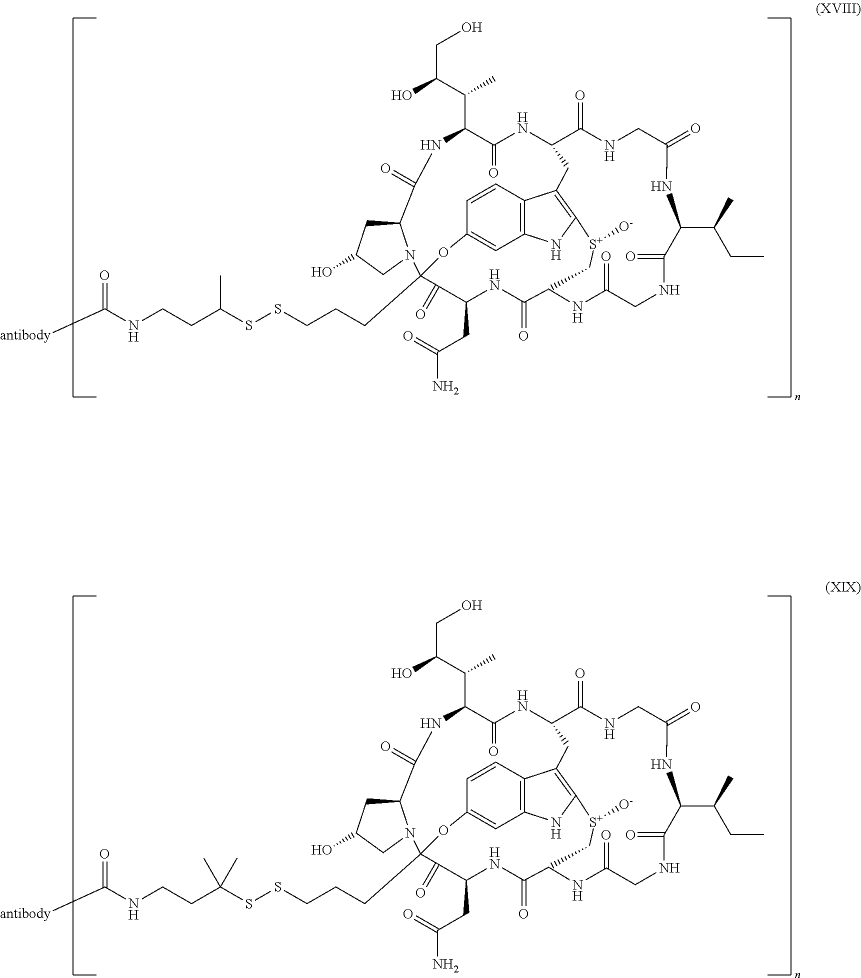 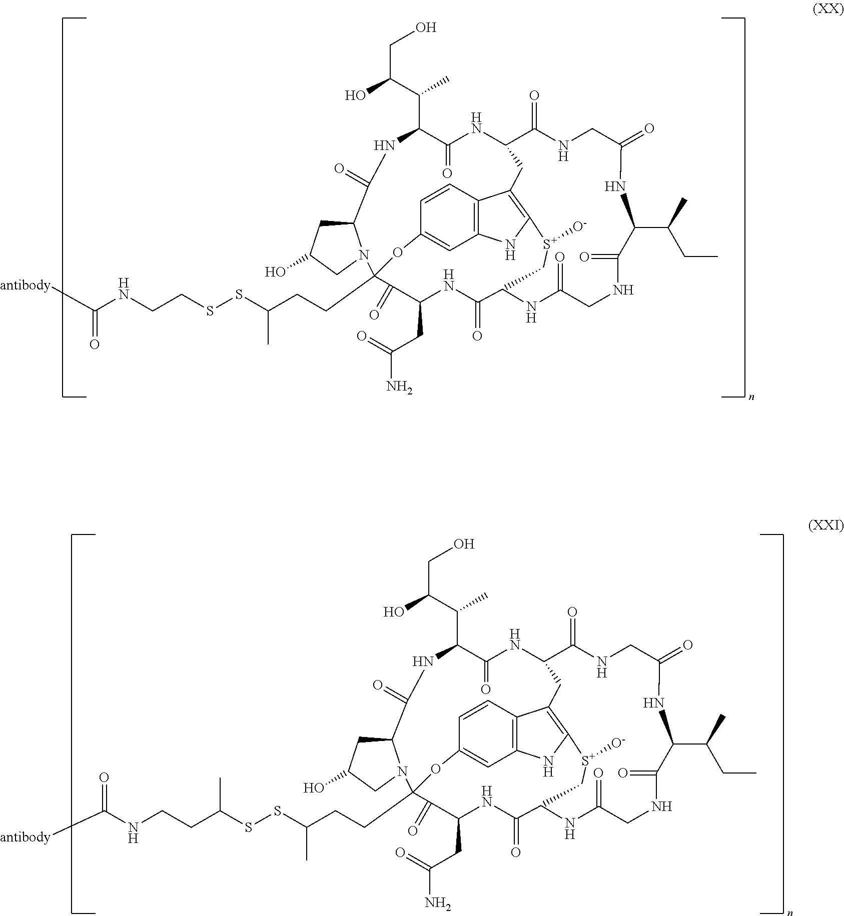 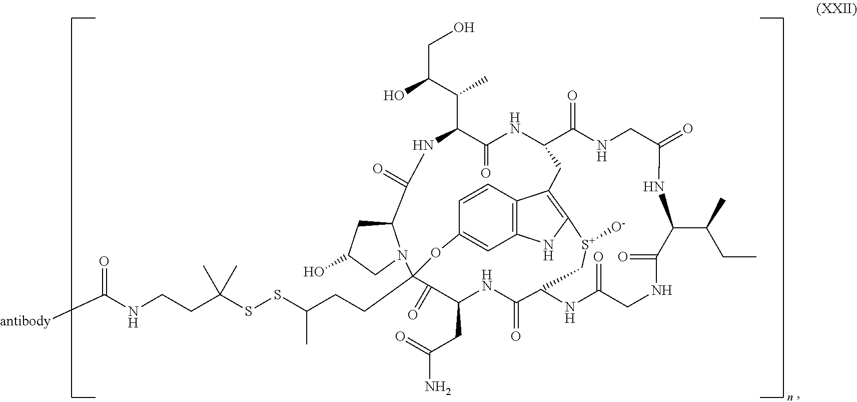
wherein said amatoxin linker moieties are coupled to ε-amino groups of naturally occurring lysine residues of said antibody, and wherein n is from 1 to 8.

10. The composition of claim 1, wherein said conjugate is a compound according to any one of formulas XXII, XXIV, XIIIb, XVb, XVIb, XVIIb, XVIIIb, XIXb, XXb, XXIb, and XXIIb: 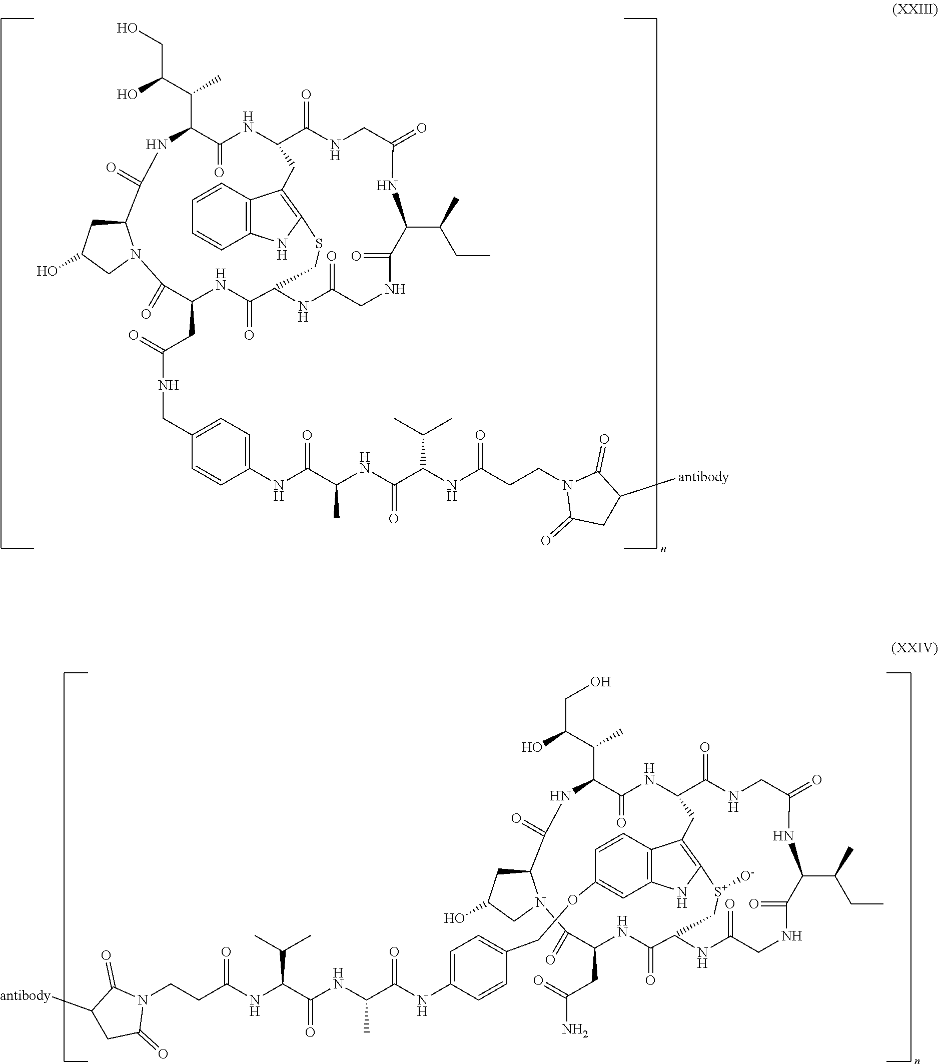 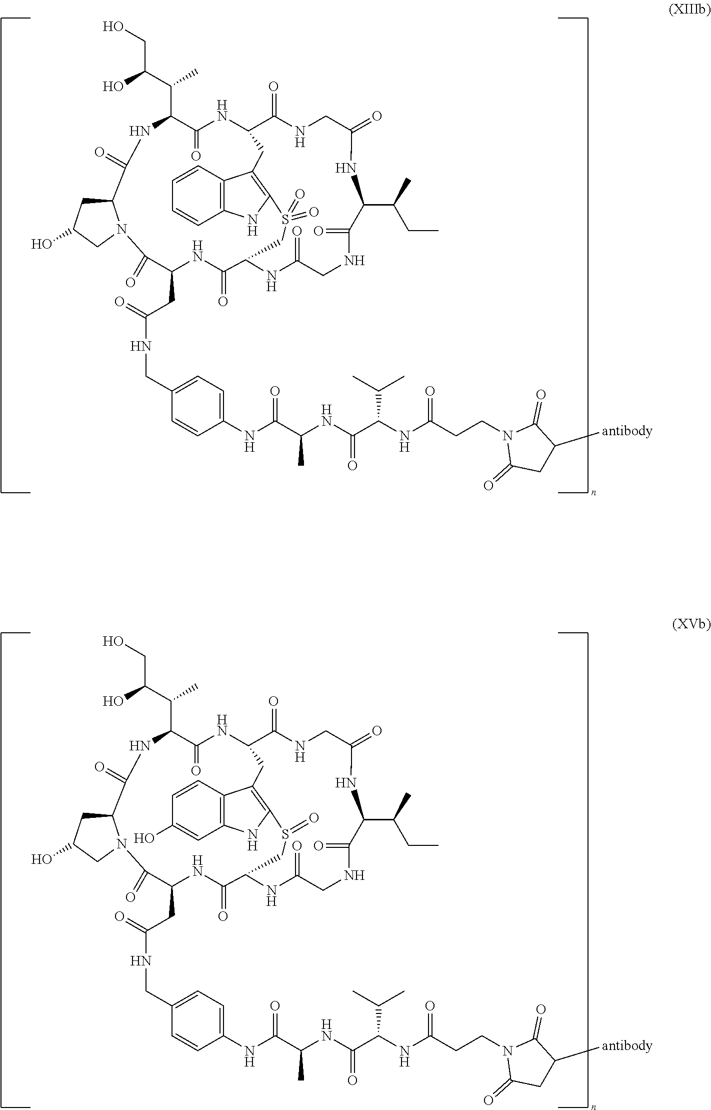 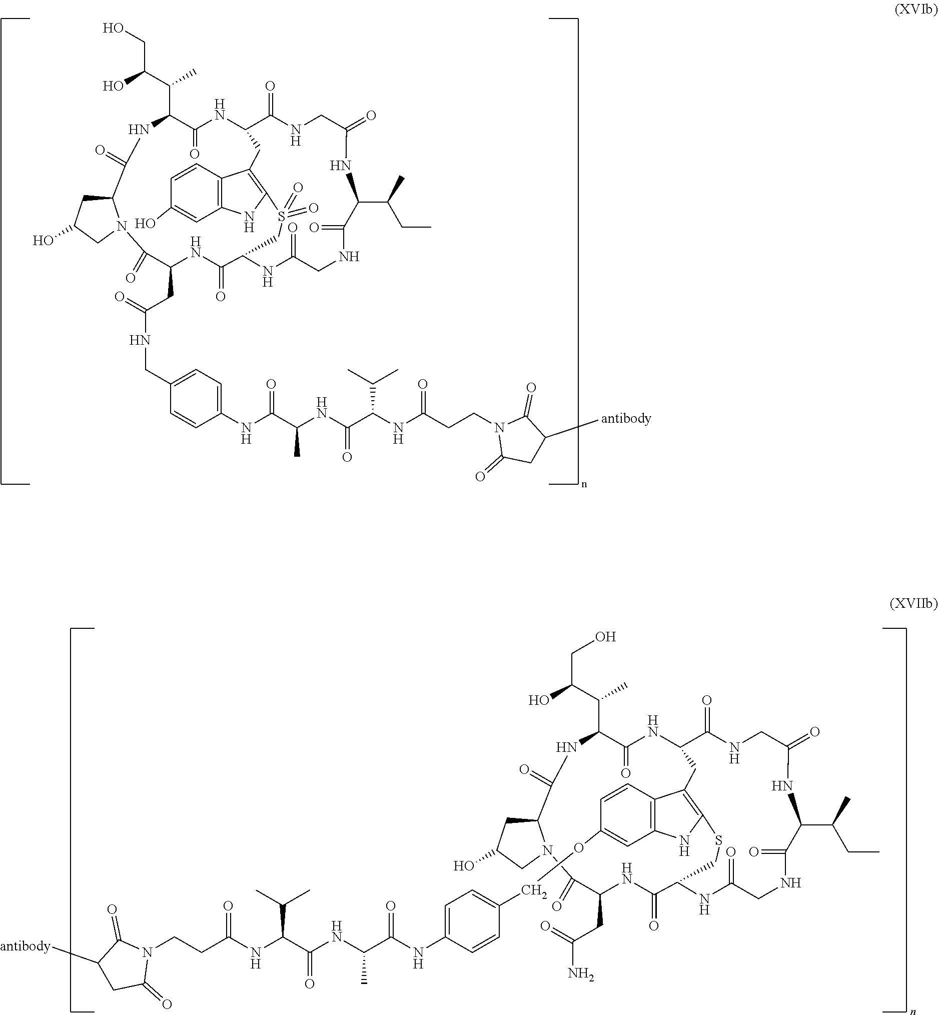 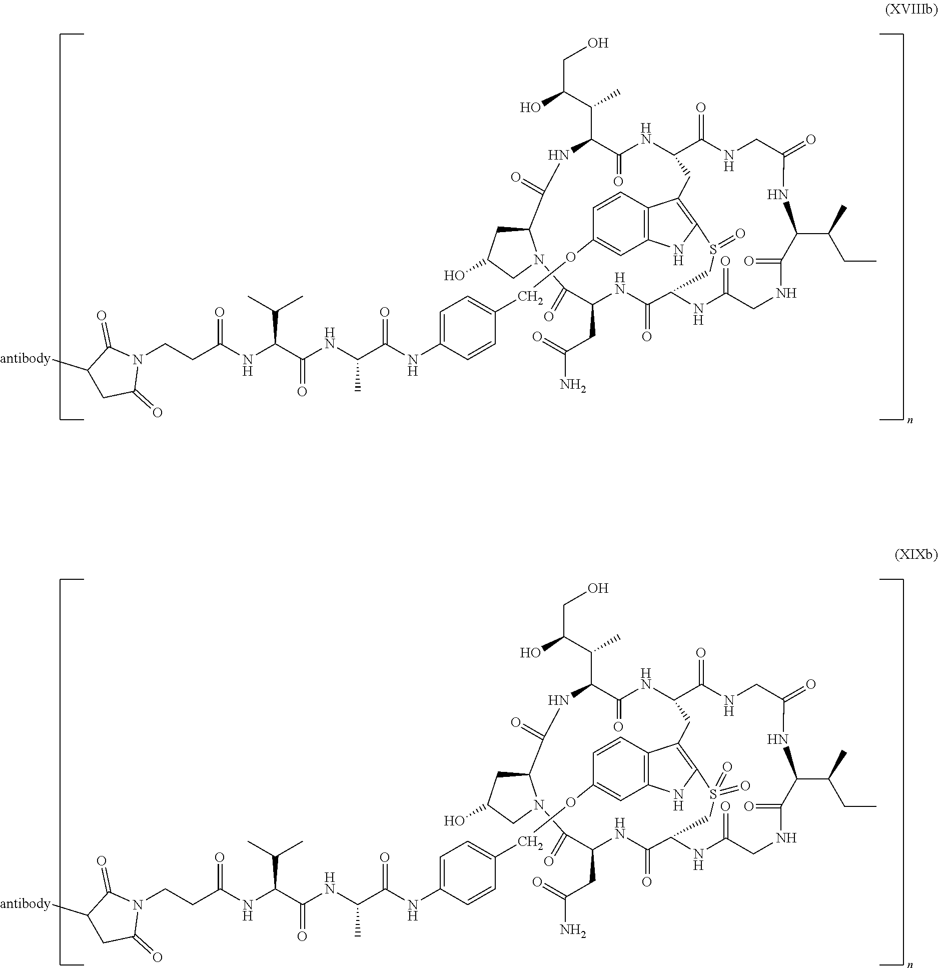 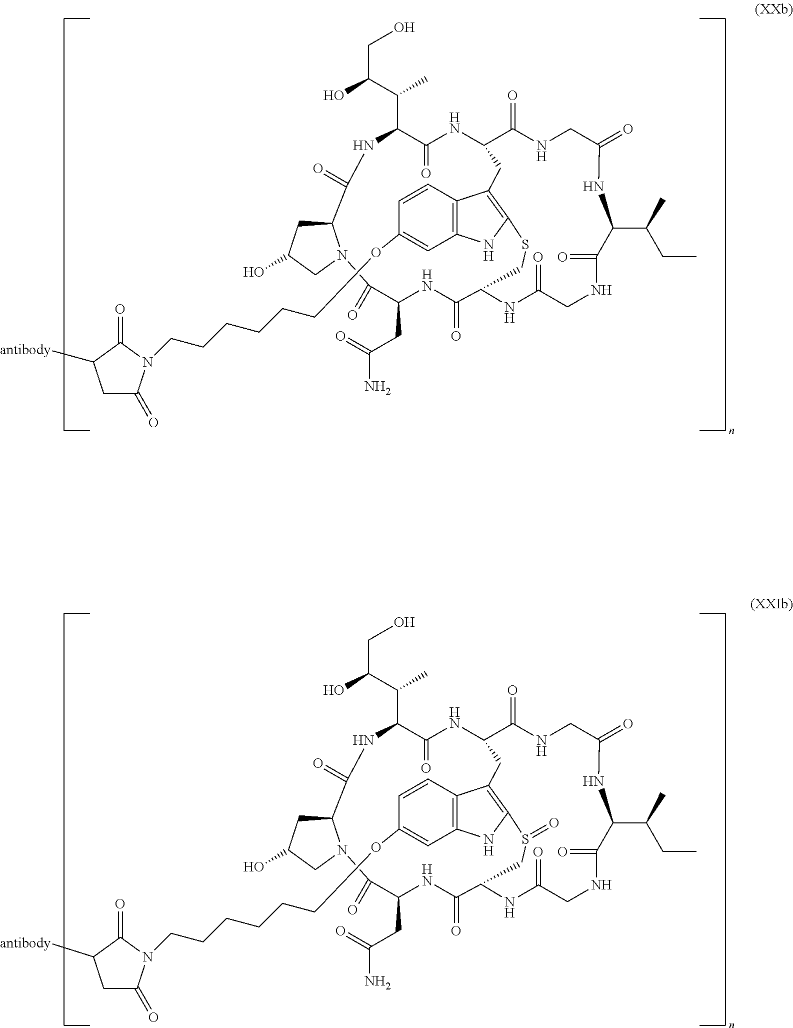 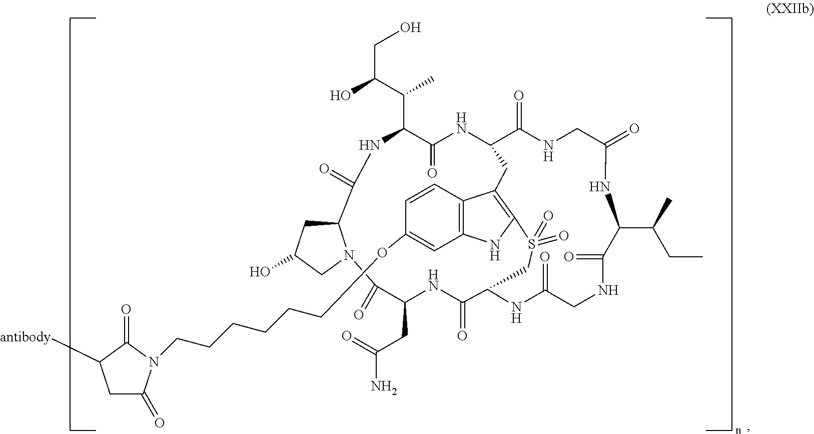
wherein said amatoxin linker moieties are coupled to the thiol groups of cysteine residues of the antibody, and wherein n is from 1 to 10.

11. A pharmaceutical formulation comprising the composition of claim 1 and one or more pharmaceutically acceptable buffers, surfactants, diluents, carriers, excipients, fillers, binders, lubricants, glidants, disintegrants, adsorbents, and/or preservatives.

12. A method for treating cancer in a human subject in need thereof, wherein the method comprises administering to the human subject the composition according to claim 1.

13. The method of claim 12, wherein said cancer is selected from the group consisting of melanoma, squamous and non-squamous non-small cell lung cancer, metastatic small cell lung cancer, renal cell carcinoma, Hodkin lymphoma, urothelial carcinoma, head and neck squamous cell carcinoma, Merkel cell carcinoma, hepatocellular carcinoma, gastric and gastroesophageal carcinoma, metastatic colorectal cancer, primary mediastinal B cell lymphoma, recurrent or metastatic cervical cancer, and metastatic cutaneous squamous cell carcinoma.

14. A method for treating cancer in a human subject in need thereof, wherein the method comprises administering to the human subject the pharmaceutical formulation according to claim 11.

15. The method of claim 14, wherein said cancer is selected from the group consisting of melanoma, squamous and non-squamous non-small cell lung cancer, metastatic small cell lung cancer, renal cell carcinoma, Hodgkin lymphoma, urothelial carcinoma, head and neck squamous cell carcinoma, Merkel cell carcinoma, hepatocellular carcinoma, gastric and gastroesophageal carcinoma, metastatic colorectal cancer, primary mediastinal B cell lymphoma, recurrent or metastatic cervical cancer, and metastatic cutaneous squamous cell carcinoma.
